(19) United States
(12) Patent Application Publication
(10) Pub. No.: US 2016/0362631 A1
(43) Pub. Date: Dec. 15, 2016

(54) Fragrance Fixatives and Compositions Comprising Thereof

(71) Applicant: The Procter & Gamble Company, Cincinnati, OH (US)

(72) Inventors: Lynette Anne Makins HOLLAND, Abbots Langley (GB); Christelle Marie Sandrine BONNET, Caillouet-Orgeville (FR); Fabienne PASTOR, Meriel (FR); Jose Maria VELAZQUEZ MENDOZA, Ascot (GB); Jonathan Richard STONEHOUSE, Windlesham (GB); William Eoghan STAITE, Egham (GB); David Thomas STANTON, Hamilton, OH (US); Oreste TODINI, Bruxelles (BE); Sarah Kyakyo Kanyunyuzi NYAKANA, Windsor (GB); Susana FERNANDEZ PRIETO, Benicassim (ES); Johan SMETS, Lubbeek (BE); Jeffrey John SCHEIBEL, Cincinnati, OH (US); Isabelle GUIMET, Brussels (BE)

(21) Appl. No.: 15/183,353

(22) Filed: Jun. 15, 2016

Related U.S. Application Data
(60) Provisional application No. 62/175,439, filed on Jun. 15, 2015.

(51) Int. Cl. C11B 9/00; A61Q 19/00; A61K 8/49; A61Q 13/00; A61K 8/37; A61K 8/34

(52) U.S. Cl. CPC C11B 9/0015; C11B 9/0061; C11B 9/0092; C11B 9/0034; C11B 9/0019; A61K 8/37; A61K 8/34; A61K 8/342; A61K 8/492; A61Q 13/00; A61Q 19/007

(57) ABSTRACT

The present invention relates to substantially non-odorous fragrance fixatives for extending the fragrance intensity or character, of fragrance materials over time. The invention also covers compositions of fragrance materials and the substantially non-odorous fragrance fixatives and methods of use of the compositions for perfuming suitable substrates, including hard surfaces and body parts, particularly skin and hair.

### FIELD OF THE INVENTION

The present invention concerns substantially non-odorous fragrance fixatives and compositions comprising said substantially non-odorous fragrance fixatives to extend the fragrance profile, preferably the fragrance intensity and/or fragrance character, of the fragrance materials over time.

### BACKGROUND OF THE INVENTION

Fragrances in some products, particularly (but not exclusively) ethanol-based ones, tend to lose their fragrance profile (i.e., character and intensity) rapidly after application. Various materials have been used to make the fragrance profile last longer. These are known as fragrance fixatives. Some substantially non-odorous examples include: (i) capsules or complexes based on dextrines, melamines or obtained by coacervation of anionic and cationic polymers, (ii) film-forming polymers, or (iii) perfume base notes such as musks. The drawbacks of capsules or complexes are that they are difficult to formulate into a fragrance composition and/or the release is little controlled but depends on variable factors like moisture or sebum amount or sweat intensity. The issue with film-forming polymers is that they produce very noticeable and undesirable films (both visual and tactile). The disadvantage of perfume base notes is that they can negatively impact the fragrance character of the compositions to which they are added.

Thus, there is a need for new fragrance fixatives to extend the fragrance profile, preferably the intensity or character, of a fragrance material to maintain its intensity over time and particularly in retaining the initial character and intensity of the characters. It is also desirable that the fragrance fixatives should not adversely affect the aromatic and/or aesthetic character of the products to which they are added.

### SUMMARY OF THE INVENTION

In one aspect, the present invention provides a substantially non-odorous fragrance fixative comprising at least one material selected from the group consisting of the materials in Table 1, as provided herein below.

In another aspect, the present invention is directed to a composition comprising a fragrance component present in an amount of from about 0.04 wt % to about 30 wt %, relative to the total weight of the composition, and at least one substantially non-odorous fragrance fixative, as described herein below, present in an amount of from about 0.1 wt % to about 20 wt %, relative to the total weight of the composition.

In yet another aspect, the present invention is further directed to a method of modifying or enhancing the odour properties of a surface with a composition of the present invention, by contacting or treating the surface with the composition.

In yet another aspect, the present invention is further directed to a composition comprising fragrance materials and a substantially non-odorous fragrance fixative according to Table 1 for extending the fragrance profile of the fragrance materials vs. a control composition absent of the substantially non-odorous fragrance fixative.

These and other features of the present invention will become apparent to one skilled in the art upon review of the following detailed description when taken in conjunction with the appended claims.

### BRIEF DESCRIPTION OF THE DRAWINGS

While the specification concludes with claims particularly pointing out and distinctly claiming the invention, it is believed that the invention will be better understood from the following description of the accompanying figures wherein:

FIG. 1 provides the panel test results of perceived fragrance profile, particularly improved fragrance intensity of Composition A comprising Dimethyl Benzyl Carbinol fragrance material and Piperonyl butoxide substantially non-odorous fragrance fixative as compared to Composition B, a control absent of a substantially non-odorous fragrance fixative (Piperonyl butoxide), and as a function of time elapsed since application of the composition.

FIG. 2 provides the panel test results of perceived fragrance profile, particularly improved fragrance intensityof Composition C comprising Eugenol fragrance material and Piperonyl butoxide substantially non-odorous fragrance fixative as compared to Composition D, a control absent of a substantially non-odorous fragrance fixative (Piperonyl butoxide), and as a function of time elapsed since application of the composition.

FIG. 3 provides the panel test results of perceived fragrance profile, particularly improved fragrance intensity of Composition I comprising Dimethyl Benzyl Carbinol fragrance material and Poly(PG)monobutyl ether substantially non-odorous fragrance fixative as compared to Composition J, a control absent of a substantially non-odorous fragrance fixative (Poly(PG)monobutyl ether), and as a function of time elapsed since application of the composition.

FIG. 4 provides the panel test results of perceived fragrance profile, particularly improved fragrance intensity of Composition K comprising Eugenol fragrance material and Poly(PG)monobutyl ether substantially non-odorous fragrance fixative as compared to Composition L, a control absent of a substantially non-odorous fragrance fixative (Poly(PG)monobutyl ether), and as a function of time elapsed since application of the composition.

FIG. 5 provides the panel test results of perceived fragrance profile, particularly improved fragrance intensity of Composition M comprising Phenethyl alcohol (PEA) fragrance material and Poly(PG)monobutyl ether substantially non-odorous fragrance fixative as compared to Composition N, a control absent of a substantially non-odorous fragrance fixative (Poly(PG)monobutyl ether), and as a function of time elapsed since application of the composition.

FIG. 6 provides the panel test results of perceived fragrance profile, particularly improved fragrance intensity of Composition Q comprising Indole fragrance material and Triglycol substantially non-odorous fragrance fixative as compared to Composition R, a control absent of a substantially non-odorous fragrance fixative (Triglycol), and as a function of time elapsed since application of the composition.

FIG. 7 provides the panel test results of perceived fragrance profile, particularly improved fragrance intensity of Composition S comprising Eugenol fragrance material and Triglycol substantially non-odorous fragrance fixative as compared to Composition T, a control absent of a substantially non-odorous fragrance fixative (Triglycol), and as a function of time elapsed since application of the composition.

FIG. 8 provides the evaporation profile results for a representative component (i.e., indole) of test composition (MOD1) comprising a volatile fragrance material mixture and Tergitol® 15-S-7 substantially non-odorous fragrance fixative as compared to a control composition (REF), and as a function of time elapsed since application of the composition.

FIG. 9 provides the evaporation profile results for a representative component (i.e., indole) of test composition (MOD2) comprising a volatile fragrance material mixture and PPG-7-Buteth-10 substantially non-odorous fragrance fixative as compared to a control composition (REF), and as a function of time elapsed since application of the composition.

FIG. 10 provides the evaporation profile results for a representative component (i.e., indole) of test composition (MOD3) comprising a volatile fragrance material mixture and Nikkol PBC-33 substantially non-odorous fragrance fixative as compared to a control composition (REF), and as a function of time elapsed since application of the composition.

FIG. 11 provides the evaporation profile results for a representative component (i.e., indole) of test composition (MOD4) comprising a volatile fragrance material mixture and Neodol 45-7 Alcohol Ethoxylate substantially non-odorous fragrance fixative as compared to a control composition (REF), and as a function of time elapsed since application of the composition.

FIG. 12 provides the evaporation profile results for a representative component (i.e., indole) of test composition (MOD5) comprising a volatile fragrance material mixture and Bio-soft N25-7 substantially non-odorous fragrance fixative as compared to a control composition (REF), and as a function of time elapsed since application of the composition.

FIG. 13 provides the evaporation profile results for a representative component (i.e., indole) of test composition (MOD6) comprising a volatile fragrance material mixture and Bio-soft N23-6.5 substantially non-odorous fragrance fixative as compared to a control composition (REF), and as a function of time elapsed since application of the composition.

FIG. 14 provides the evaporation profile results for a representative component (i.e., indole) of test composition (MOD7) comprising a volatile fragrance material mixture and Cremophor® A 25 substantially non-odorous fragrance fixative as compared to a control composition (REF), and as a function of time elapsed since application of the composition.

FIG. 15 provides the evaporation profile results for a representative component (i.e., indole) of test composition (MODE) comprising a volatile fragrance material mixture and Bio-soft N91-8 substantially non-odorous fragrance fixative as compared to a control composition (REF), and as a function of time elapsed since application of the composition.

FIG. 16 provides the evaporation profile results for a representative component (i.e., indole) of test composition (MOD9) comprising a volatile fragrance material mixture and Genapol® C-100 substantially non-odorous fragrance fixative as compared to a control composition (REF), and as a function of time elapsed since application of the composition.

FIG. 17 provides the evaporation profile results for a representative component (i.e., indole) of test composition (MOD10) comprising a volatile fragrance material mixture and Rhodasurf® LA 30 substantially non-odorous fragrance fixative as compared to a control composition (REF), and as a function of time elapsed since application of the composition.

FIG. 18 provides the evaporation profile results for a representative component (i.e., indole) of test composition (MOD11) comprising a volatile fragrance material mixture and Poly(ethylene glycol) methyl ether substantially non-odorous fragrance fixative as compared to a control composition (REF), and as a function of time elapsed since application of the composition.

FIG. 19 provides the evaporation profile results for a representative component (i.e., indole) of test composition (MOD12) comprising a volatile fragrance material mixture and Arlamol™ PS11E substantially non-odorous fragrance fixative as compared to a control composition (REF), and as a function of time elapsed since application of the composition.

FIG. 20 provides the evaporation profile results for a representative component (i.e., indole) of test composition (MOD13) comprising a volatile fragrance material mixture and Brij® S100 substantially non-odorous fragrance fixative as compared to a control composition (REF), and as a function of time elapsed since application of the composition.

FIG. 21 provides the evaporation profile results for a representative component (i.e., indole) of test composition (MOD14) comprising a volatile fragrance material mixture and Brij® C-58 substantially non-odorous fragrance fixative as compared to a control composition (REF), and as a function of time elapsed since application of the composition.

FIG. 22 provides the evaporation profile results for a representative component (i.e., indole) of test composition (MOD15) comprising a volatile fragrance material mixture and Pluronic® F-127 substantially non-odorous fragrance fixative as compared to a control composition (REF), and as a function of time elapsed since application of the composition.

FIG. 23 provides the evaporation profile results for a representative component (i.e., indole) of test composition (MOD16) comprising a volatile fragrance material mixture and Bio-soft N1-5 substantially non-odorous fragrance fixative as compared to a control composition (REF), and as a function of time elapsed since application of the composition.

FIG. 24 provides the evaporation profile results for a representative component (i.e., indole) of test composition (MOD17) comprising a volatile fragrance material mixture and Polyoxyethylene (10) lauryl ether substantially non-odorous fragrance fixative as compared to a control composition (REF), and as a function of time elapsed since application of the composition.

FIG. 25 provides the evaporation profile results for a representative component (i.e., indole) of test composition (MOD18) comprising a volatile fragrance material mixture and Arlamol™ PC10 substantially non-odorous fragrance fixative as compared to a control composition (REF), and as a function of time elapsed since application of the composition.

FIG. 26 provides the evaporation profile results for a representative component (i.e., indole) of test composition (MOD19) comprising a volatile fragrance material mixture and Poly(ethylene glycol) (18) tridecyl ether substantially non-odorous fragrance fixative as compared to a control composition (REF), and as a function of time elapsed since application of the composition.

FIG. 27 provides the evaporation profile results for a representative component (i.e., indole) of test composition (MOD20) comprising a volatile fragrance material mixture and ALFONIC® 10-8 Ethoxylate substantially non-odorous fragrance fixative as compared to a control composition (REF), and as a function of time elapsed since application of the composition.

FIG. 28 provides the evaporation profile results for a representative component (i.e., indole) of test composition (MOD21) comprising a volatile fragrance material mixture and Brij® 020-SS substantially non-odorous fragrance fixative as compared to a control composition (REF), and as a function of time elapsed since application of the composition.

FIG. 29 provides the evaporation profile results for a representative component (i.e., indole) of test composition (MOD22) comprising a volatile fragrance material mixture and Diethylene glycol butyl ether substantially non-odorous fragrance fixative as compared to a control composition (REF), and as a function of time elapsed since application of the composition.

FIG. 30 provides the evaporation profile results for a representative component (i.e., indole) of test composition (MOD23) comprising a volatile fragrance material mixture and Ethylene glycol monohexadecyl ether substantially non-odorous fragrance fixative as compared to a control composition (REF), and as a function of time elapsed since application of the composition.

FIG. 31 provides the evaporation profile results for a representative component (i.e., indole) of test composition (MOD24) comprising a volatile fragrance material mixture and Poly(propylene glycol) monobutyl ether substantially non-odorous fragrance fixative as compared to a control composition (REF), and as a function of time elapsed since application of the composition.

FIG. 32 provides the evaporation profile results for a representative component (i.e., indole) of test composition (MOD25) comprising a volatile fragrance material mixture and Dowanol™ TPnB substantially non-odorous fragrance fixative as compared to a control composition (REF), and as a function of time elapsed since application of the composition.

FIG. 33 provides the evaporation profile results for a representative component (i.e., indole) of test composition (MOD26) comprising a volatile fragrance material mixture and Tripropylene Glycol substantially non-odorous fragrance fixative as compared to a control composition (REF), and as a function of time elapsed since application of the composition.

FIG. 34 provides the evaporation profile results for a representative component (i.e., indole) of test composition (MOD27) comprising a volatile fragrance material mixture and Cithrol™ substantially non-odorous fragrance fixative as compared to a control composition (REF27), and as a function of time elapsed since application of the composition.

FIG. 35 provides the evaporation profile results for a representative component (i.e., indole) of test composition (MOD28) comprising a volatile fragrance material mixture and Igepal® CO-630 substantially non-odorous fragrance fixative as compared to a control composition (REF), and as a function of time elapsed since application of the composition.

FIG. 36 provides the evaporation profile results for a representative component (i.e., indole) of test composition (MOD29) comprising a volatile fragrance material mixture and Nikkol Decaglyn 3-OV substantially non-odorous fragrance fixative as compared to a control composition (REF), and as a function of time elapsed since application of the composition.

FIG. 37 provides the evaporation profile results for a representative component (i.e., indole) of test composition (MOD30) comprising a volatile fragrance material mixture and NIKKOL Hexaglyn 1-L substantially non-odorous fragrance fixative as compared to a control composition (REF), and as a function of time elapsed since application of the composition.

FIG. 38 provides the evaporation profile results for a representative component (i.e., indole) of test composition (MOD31) comprising a volatile fragrance material mixture and Emalex CS-10 substantially non-odorous fragrance fixative as compared to a control composition (REF), and as a function of time elapsed since application of the composition.

FIG. 39 provides the evaporation profile results for a representative component (i.e., indole) of test composition (MOD32) comprising a volatile fragrance material mixture and Dioctyl ether substantially non-odorous fragrance fixative as compared to a control composition (REF), and as a function of time elapsed since application of the composition.

FIG. 40 provides the evaporation profile results for a representative component (i.e., indole) of test composition (MOD33) comprising a volatile fragrance material mixture and Jeecol CA-10 substantially non-odorous fragrance fixative as compared to a control composition (REF), and as a function of time elapsed since application of the composition.

FIG. 41 provides the evaporation profile results for a representative component (i.e., indole) of test composition (MOD34) comprising a volatile fragrance material mixture and Steareth-10 substantially non-odorous fragrance fixative as compared to a control composition (REF), and as a function of time elapsed since application of the composition.

FIG. 42 provides the evaporation profile results for a representative component (i.e., indole) of test composition (MOD35) comprising a volatile fragrance material mixture and Nonaethylene glycol monododecyl ether substantially non-odorous fragrance fixative as compared to a control composition (REF), and as a function of time elapsed since application of the composition.

FIG. 43 provides the evaporation profile results for a representative component (i.e., indole) of test composition (MOD36) comprising a volatile fragrance material mixture and Glycerol propoxylate substantially non-odorous fragrance fixative as compared to a control composition (REF), and as a function of time elapsed since application of the composition.

FIG. 44 provides the evaporation profile results for a representative component (i.e., indole) of test composition (MOD37) comprising a volatile fragrance material mixture and Glycerol ethoxylate substantially non-odorous fragrance fixative as compared to a control composition (REF), and as a function of time elapsed since application of the composition.

FIG. 45 provides the evaporation profile results for a representative component (i.e., indole) of test composition (MOD38) comprising a volatile fragrance material mixture and Hexaethylene glycol monohexadecyl ether substantially non-odorous fragrance fixative as compared to a control composition (REF), and as a function of time elapsed since application of the composition.

FIG. 46 provides the evaporation profile results for a representative component (i.e., indole) of test composition (MOD39) comprising a volatile fragrance material mixture and Aquaflex™ XL-30 substantially non-odorous fragrance fixative as compared to a control composition (REF), and as a function of time elapsed since application of the composition.

FIG. 47 provides the evaporation profile results for a representative component (i.e., indole) of test composition (MOD40) comprising a volatile fragrance material mixture and Piperonyl Butoxide substantially non-odorous fragrance fixative as compared to a control composition (REF), and as a function of time elapsed since application of the composition.

FIG. 48 provides the evaporation profile results for a representative component (i.e., indole) of test composition (MOD41) comprising a volatile fragrance material mixture and Diphenhydramine HCl substantially non-odorous fragrance fixative as compared to a control composition (REF), and as a function of time elapsed since application of the composition.

FIG. 49 provides the evaporation profile results for a representative component (i.e., indole) of test composition (MOD42) comprising a volatile fragrance material mixture and Di(propylene glycol) propyl ether substantially non-odorous fragrance fixative as compared to a control composition (REF), and as a function of time elapsed since application of the composition.

FIG. 50 provides the evaporation profile results for a representative component (i.e., indole) of test composition (MOD43) comprising a volatile fragrance material mixture and Poly(melamine-co-formaldehyde) methylated substantially non-odorous fragrance fixative as compared to a control composition (REF), and as a function of time elapsed since application of the composition.

### DETAILED DESCRIPTION OF THE INVENTION

### Definitions

As used herein, articles such as “a” and “an” when used in a claim, are understood to mean one or more of what is claimed or described.

As used herein, the terms “include”, “includes” and “including” are meant to be non-limiting.

As used herein, the term “body splash” means a body care formulation that is applied to the body. Typically, the body splash is applied to the body after bathing and provides a subtle hint of scent to the body. Body splashes are commonly used by consumers who prefer less strong fragrance compositions. A body splash may comprise an ethanol-free composition according to the present invention which comprises from 0.2-8 wt %, relative to the total weight of the composition, of a fragrance component. The body splash may further comprise alkyl polyglucosides as non-ionic surfactants.

As used herein, the term “body spray” means a formulation comprising fragrance materials intended to be applied to the body to prevent or mask body odor caused by the bacterial breakdown of perspiration on the body (e.g., armpits, feet, and other areas of the body). The body spray may also provide a fragrance expression to the consumers. Typically, body spray compositions are applied as an aerosol spray in an effective amount on the skin of a consumer.

As used herein, the term “composition” includes a fine fragrance composition intended for application to a surface, such as for example, body surface like skin or hair, i.e., to impart a pleasant odour thereto, or cover a malodour thereof. They are generally in the form of perfume concentrates, perfumes, parfums, eau de parfums, eau de toilettes, aftershaves, or colognes. The fine fragrance compositions may be an ethanol-based composition. The term “composition” may also include a cosmetic composition, which comprises a fragrance material for the purposes of delivering a pleasant smell to drive consumer acceptance of the cosmetic composition. The term “composition” may also include body splashes or body sprays. The term “composition” may also include cleaning compositions, such as fabric care composition or home care compositions, including air care compositions (e.g., air fresheners), for use on clothing or other substrates such as hard surfaces (e.g., dishes, floors, countertops). Additional non-limiting examples of “composition” may also include facial or body powder, foundation, deodorant, body/facial oil, mousse, creams (e.g., cold creams), waxes, sunscreens and blocks, bath and shower gels, lip balms, self-tanning compositions, masks and patches.

As used herein, the term “consumer” means both the user of the composition and the observer nearby or around the user.

As used herein, the terms “fragrance” and “perfume” are used interchangeably to designate the component in the composition that is formed of fragrance materials, i.e., ingredients capable of imparting or modifying the odour of skin or hair or other substrate.

As used herein, the term “fragrance material” and “fragrance materials” relates to a perfume raw material, or a mixture of perfume raw materials, that are used to impart an overall pleasant odour or fragrance profile to a composition. “Fragrance materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also know for use as “fragrance materials”. The individual perfume raw materials which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, N.J., USA and more recently re-published by Allured Publishing Corporation Illinois (1994). Additionally, some perfume raw materials are supplied by the fragrance houses (Firmenich, International Flavors & Fragrances, Givaudan, Symrise) as mixtures in the form of proprietary speciality accords. Non-limiting examples of the fragrance materials useful herein include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH-change, or by enzymatic release.

As used herein, the term “fragrance profile” means the description of how the fragrance is perceived by the human nose at any moment in time. The fragrance profile may change over time. It is a result of the combination of the low volatile fragrance materials and the volatile fragrance materials, if present, of a fragrance. A fragrance profile is composed of 2 characteristics: ‘intensity’ and ‘character’. The ‘intensity’ relates to the perceived strength whilst ‘character’ refers to the odour impression or quality of the perfume, i.e., fruity, floral, woody, etc.

As used herein, the terms “fixative” and “fragrance fixative” are used interchangeably to designate an agent having the capacity to affect the fragrance profile, such as for example, by impacting the fragrance materials' evaporation rate. The fixative may mediate its effect by lowering the vapor pressure of the fragrance materials and increasing their adherence to the substrate (skin and/or hair) thus ensuring a longer-lasting impression of the fragrance. Suitable examples of the fixative are provided herein below, particularly in Table 1.

As used herein, the term “substantially non-odorous” means an agent that does not impart an odour of its own when added into a composition of the present invention. For example, a “substantially non-odorous fragrance fixative” does not impart a new odour that alters the character of the fragrance profile of the composition to which it is added. The term “substantially non-odorous” also encompasses an agent that may impart a minimal or slight odour of its own when added into a composition of the present invention. However, the odour imparted by the “substantially non-odorous fragrance fixative” is generally undetectable or tends to not substantively alter the character of the fragrance profile of the composition to which it is added initially or preferably over time. Furthermore, the term “substantially non-odorous” also includes materials that are perceivable only by a minority of people or those materials deemed anosmic to the majority of people. Furthermore, the term “substantially non-odorous” also includes materials that may, from particular suppliers, contain an odour due to impurities, such as when the materials contain the impurities at not more than about 5 wt %, preferably not more than 1 wt %, often even not more than 1 part per million (ppm). These impurities maybe removed by purification techniques known in the art as required to make them suitable for use in fragrance compositions of the present invention.

As used herein, the term “vapor pressure” means the partial pressure in air at a defined temperature (e.g., 25° C.) and standard atmospheric pressure (e.g., 760 mmHg or 101.325 kPa) for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a fragrance material which is defined in an open environment where material is leaving the system. The vapor pressure is determined according to the reference program Advanced Chemistry Development (ACD/Labs) Software Version 14.02, or preferably the latest version update).

It is understood that the test methods that are disclosed in the Test Methods Section of the present application must be used to determine the respective values of the parameters of Applicants' inventions as described and claimed herein.

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

Substantially Non-Odorous Fragrance Fixatives The inventors have discovered new agents that can be used as substantially non-odorous fragrance fixatives, as described herein below, to enhance or improve the fragrance profile, preferably the intensity or character, of the fragrance material. Preferable examples of the substantially non-odorous fragrance fixatives are provided in Table 1 below.

Preferably, the substantially non-odorous fragrance fixative is present in an amount of from about 0.1 wt % to about 20 wt %, preferably from about 0.5 wt % to about 18 wt % or more preferably from about 2.5 wt % to about 15 wt % or combinations thereof, relative to the total weight of the composition. Alternatively, the substantially non-odorous fragrance fixative is present in an amount of from about 0.1 wt %, 0.5 wt % or 2.5 wt % to about 15 wt %, 18 wt % or 20 wt %, relative to the total weight of the composition. If there is more than one substantially non-odorous fragrance fixatives, then the ranges provided hereinabove cover the total of all of the substantially non-odorous fragrance fixatives.

The substantially non-odorous fragrance fixatives of the present invention may be a liquid at temperatures lower than 100° C., preferably at ambient temperature. The substantially non-odorous fragrance fixatives may be fully miscible with the fragrance materials to form a single phase liquid. However, if the fragrance materials are not entirely miscible, or are immiscible, then co-solvents (e.g., dipropylene glycol (DPG), triethyl citrate, or others as well known to those skilled in the art) can be added to aid in the solubility of the fragrance materials.

Preferably, the composition according to the present invention, wherein the substantially non-odorous fragrance fixatives and fragrance component are present in a weight ratio from about 10:1 to about 1:10, preferably from about 5:1 to about 1:5, or preferably from about 3:1 to about 1:3.

The inventors have discovered that the substantially non-odorous fragrance fixatives can extend the fragrance intensity of the fragrance material over time, preferably over long periods of time such as for example, 1 hour, 2 hours, 3 hours, 4 hours, 5 hours, 6 hours, 7 hours, 8 hours, 10 hours, and possibly all the way up to 24 hrs after application as compared to controls, i.e., compositions containing no substantially non-odorous fragrance fixatives.

Additionally, the inventors have discovered that the substantially non-odorous fragrance fixatives can extend the fragrance character, preferably the portion of the fragrance profile attributable to the volatile fragrance materials. By “extend” it is meant that the fragrance profile of the composition, preferably the components contributed by the volatile fragrance materials, can be pereceived by the consumer at later time points such as for example, 1 hour, 2 hours, 3 hours, 4 hours, 5 hours, 6 hours, 7 hours, 8 hours, 10 hours, and possibly all the way up to 24 hrs after application as compared to controls, i.e., compositions containing no substantially non-odorous fragrance fixatives.

### Compositions

In one aspect, the present invention provides for a composition comprising a fragrance component present in an amount of from about 0.04 wt % to 30 wt %, preferably 1 wt % to about 30 wt %, more preferably less than about 25 wt %, yet more preferably less than about 20 wt %, yet even more preferably less than about 15 wt %, yet even more preferably less than about 10 wt % or most preferably less than about 8 wt %, relative to the total weight of the composition. Alternatively, the fragrance component is present in an amount of from about 0.04 wt %, 0.3 wt %, 1 wt %, 2 wt %, 5 wt %, 8 wt % or 10 wt %, to about 15 wt %, 20 wt %, 25 wt % or 30 wt %, relative to the total weight of the composition.

Preferably, the compositions of the present invention comprise:
(i) a fragrance component present in an amount of from about 0.04 wt % to about 30 wt %, relative to the total weight of the composition; and
(ii) at least one substantially non-odorous fragrance fixative from the group consisting of the materials in Table 1, wherein the substantially non-odorous fragrance fixative is present in the amount of from about 0.1 wt % to about 20 wt %, relative to the total weight of the composition.

Preferably, the composition of the present invention, wherein:
(i) fragrance component is present preferably from about 0.04 wt %, 0.1 wt %, 0.5 wt %, 1 wt % or 2 wt % to about 30 wt %, 25 wt %, 20 wt %, 15 wt %, 10 wt % or 8 wt %, relative to the total weight of the composition, and wherein the fragrance component comprises:
   (a) at least one low volatile fragrance material having a vapor pressure less than 0.001 Torr (0.000133 kPa) at 25° C.; and
   (b) the low volatile fragrance material is present in an amount of less than about 30 wt %, or less than about 28 wt %, or less than about 25 wt %, relative to the total weight of the fragrance component; and
(ii) at least one substantially non-odorous fragrance fixative present in the amount of preferably from about 0.1 wt % to about 20 wt %, or preferably from about 0.5 wt % to about 18 wt %, or more preferably from about 2.5 wt % to about 15 wt %, relative to the total weight of the composition.

Preferably, the composition of the present invention, wherein the low volatile fragrance material is present in an amount of from about 10 wt % to about 30 wt %, relative to the total weight of the fragrance component.

Preferably, the present invention relates to a fine fragrance composition, preferably in the form of of a perfume concentrate, a perfume, a parfum, an eau de toilette, an eau de parfum, or a cologne.

Preferably, the present invention relates to a composition, wherein the composition is in the form of a body splash or a body spray.

Therefore, it goes without saying that the compositions of the present invention encompasses any composition comprising any of the ingredients cited herein, in any embodiment wherein each such ingredient is independently present in any appropriate amount as defined herein. Many such compositions, than what is specifically set out herein, can be encompassed.

### Entrapment Materials

In yet another aspect, compositions of the present invention may comprise an entrapment material at a level such that the weight ratio of the entrapment material to the fragrance materials is in the range of from about 1:20 to about 20:1. Preferably, the composition may comprise an entrapment material present in the amount of from about 0.001 wt % to about 40 wt %, from about 0.1 wt % to about 25 wt %, from about 0.3 wt % to about 20 wt %, from about 0.5 wt % to about 10 wt %, or from about 0.75 wt % to about 5 wt %, relative to the total weight of the composition. The compositions disclosed herein may comprise from 0.001 wt % to 40%, from 0.1 wt % to 25 wt %, from 0.3 wt % to 20 wt %, from 0.5 wt % to 10 wt % or from 0.75 wt % to 5 wt %, relative to the total weight of the composition, of a cyclic oligosaccharide.

Suitable entrapment materials for use herein are selected from polymers; capsules, microcapsules and nanocapsules; liposomes, absorbents; cyclic oligosaccharides and mixtures thereof. Preferred are absorbents and cyclic oligosaccharides and mixtures thereof. Highly preferred are cyclic oligosaccharides (see PCT Publication Nos. WO2000/67721 (Procter & Gamble); and WO2000/67720 (Procter & Gamble); and U.S. Pat. No. 6,893,647 (Procter & Gamble)).

### Volatile Solvents

In yet another aspect, the present invention provides the solution to the problem of extending the longevity of the fragrance profile of compositions, particularly fine fragrance and cosmetic compositions, preferably fine fragrance compositions, which commonly contain high levels of a volatile solvent. Preferably, the composition according to the present invention, further comprising a volatile solvent present in the amount of from about 10 wt %, 20 wt %, 30 wt %, 40 wt % or 50 wt % to about 90 wt %, 80 wt %, 70 wt % or 60 wt %, relative to the total weight of the composition, and wherein the solvent is a branch or unbranched C₁ to C₁₀ alkyl, akenyl or alkynyl having at least one alcohol moiety, preferably ethanol, or isopropanol, or other alcohols (e.g., methanol, propanol, isopropanol, butanol, and mixtures thereof) commonly found in commercial fine fragrance products.

Accordingly, ethanol may be present in any of the compositions of the present invention, and more specifically, it will form from about 10 wt % to about 80 wt %, or even from about 25 wt % to about 75 wt % of the composition, or combinations thereof, relative to the total weight of the composition. Alternatively, ethanol may be present in an amount of from about 10 wt % or 25 wt % to about 75 wt % or 80 wt %, relative to the total weight of the composition. The ethanol useful in the present invention may be any acceptable quality of ethanol, compatible and safe for the specific intended use of the composition such as, for example, topical applications of fine fragrance or cosmetic compositions.

### Non-Volatile Solvents

The composition may comprise a non-volatile solvent or a mixture of non-volatile solvents. Non-limiting examples of non-volatile solvents include benzyl benzoate, diethyl phthalate, isopropyl myristate, propylene glycol, dipropylene glycol, triethyl citrate, and mixtures thereof. These solvents often are introduced to the product via the perfume oil as many perfume raw materials may be purchased as a dilution in one of these solvents. Where non-volatile solvents are present, introduced either with the perfume materials or separately, then for the purposes of calculating the proportion of fragrance component having a vapor pressure of less than 0.001 Torr (0.000133 kPa) at 25° C. the total fragrance components does not include non-volatile solvents. Where non-volatile solvents are present, introduced either with the perfume materials or separately, then for the purposes of calculating the total level of fragrance component this does not include non-volatile solvents. In addition if present with cyclic oligosacchrides, the non-volatile solvent may be included at a weight ratio of the non-volatile solvent to the cyclic oligosaccharide of less than 1:1, less than 1:2, less than 1:10, or less than 1:100.

### Water

In yet another aspect, water may be present in any of the compositions of the present invention, and more specifically, it shall not exceed about 40 wt %, preferably about 20 wt % or less, or more preferably about 10 wt % or less, relative to the total weight of the composition. Alternatively, water may be present in an amount of from about 10 wt % or about 20 wt % to about 40 wt %, relative to the total weight of the composition. When the composition is a cosmetic composition the level of water should not be so high that the product becomes cloudy thus negatively impacting the product aesthetics. It is understood that the amount of water present in the composition may be from the water present in the volatile solvent (e.g., ethanol) used in the composition, as the case may be.

### Propellants

The compositions described herein may include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42% by weight of the total fill of materials stored within the container.

### Antiperspirant Active

The compositions described herein may be free of, substantially free of, or may include an antiperspirant active (i.e., any substance, mixture, or other material having antiperspirant activity). Examples of antiperspirant actives include astringent metallic salts, like the inorganic and organic salts of aluminum, zirconium and zinc, as well as mixtures thereof. Such antiperspirant actives include, for example, the aluminum and zirconium salts, such as aluminum halides, aluminum hydroxyhalides, zirconyl oxyhalides, zirconyl hydroxyhalides, and mixtures thereof.

### Other Ingredients

In yet another aspect, the composition consists essentially of the recited ingredients but may contain small amounts (not more than about 10 wt %, preferably no more than 5 wt %, or preferably no more than 2 wt % thereof, relative to the total weight of the composition) of other ingredients that do not impact on the fragrance profile, particularly the evaporation rate and release of the fragrance materials. For example, a fine fragrance composition may comprise stabilizing or anti-oxidant agents, UV filters or quenchers, or colouring agents, commonly used in perfumery.

In yet another aspect, the composition of the present invention, depending on its intended use, is a mixture of fragrance materials possibly together with other ingredients such as, for example, perfume carriers. By the term “perfume carrier”, it is meant to include materials which are practically neutral from a perfumery point of view, i.e., which does not significantly alter the organoleptic properties of perfuming components. The perfume carrier may be a compatible liquid or solid fillers, diluents, and the like. The term “compatible”, as used herein, means that the components of the compositions of this invention are capable of being combined with the primary actives of the present invention, and with each other, in a manner such that there is no interaction which would substantially reduce the efficacy of the composition under ordinary use situations. The type of carrier utilized in the present invention depends on the type of product desired and may comprise, but are not limited to, solutions, aerosols, emulsions (including oil-in-water or water-in-oil), gels, and liposomes. Preferably, the carrier is a liquid and will be a solvent such as, for example, dipropyleneglycol, diethyl phthalate, isopropyl myristate, benzyl benzoate, 2-(2-ethoxyethoxy)-1-ethanol, or ethyl citrate (triethyl citrate).

In yet another aspect, the compositions for use in the present invention may take any form suitable for use, more preferably for perfumery or cosmetic use. These include, but are not limited to, vapor sprays, aerosols, emulsions, lotions, liquids, creams, gels, sticks, ointments, pastes, mousses, powders, granular products, substrates, cosmetics (e.g., semi-solid or liquid makeup, including foundations) and the like. Preferably the compositions for use in the present invention take the form of a vapor spray. Compositions of the present invention can be further added as an ingredient to other compositions, preferably fine fragrance or cosmetic compositions, in which they are compatible. As such they can be used within solid composition or applied substrates etc.

### Article of Manufacture

The composition may be included in an article of manufacture comprising a spray dispenser. The spray dispenser may comprise a vessel for containing the composition to be dispensed. The spray dispenser may comprise an aerosolized composition (i.e. a composition comprising a propellant) within the vessel as well. Other non-limiting examples of spray dispensers include non-aerosol dispensers (e.g. vapor sprays), manually activated dispensers, pump-spray dispensers, or any other suitable spray dispenser available in the art.

### Methods of Using the Compositions

The composition of the present invention according to any embodiments described herein is a useful perfuming composition, which can be advantageously used as consumer products intended to perfume any suitable substrate or surface. As used herein, the term “substrate” means any surface to which the composition of the present invention may be applied to without causing any undue adverse effect. For example, this can include a wide range of surfaces including human or animal skin or hair, paper (fragranced paper), air in a room (air freshener or aromatherapy composition), fabric, furnishings, dishes, hard surfaces and related materials. Preferred substrates include body surfaces such as, for example, hair and skin, most preferably skin.

The composition of the present invention may be used in a conventional manner for fragrancing a substrate. An effective amount of the composition, typically from about 1 μL to about 10,000 μL, preferably from about 10 μL to about 1,000 μL, more preferably from about 25 μL to about 500 μL, or most preferably from about 50 μL to about 100 μL, or combinations thereof, is applied to the suitable substrate. Alternatively, an effective amount of the composition of the present invention is from about 1 μL, 10 μL, 25 μL or 50 μL to about 100 μL, 500 μL, 1,000 μL or 10,000 μL. The composition may be applied by hand or applied utilizing a delivery apparatus such as, for example, vaporizer or atomizer. Preferably, the composition is allowed to dry after its application to the substrate. The scope of the present invention should be considered to cover one or more distinct applications of the composition or the continuous release of a composition via a vaporizer or other type of atomizer.

The present invention provides a method of modifying or enhancing the odour properties of a body surface, preferably hair or skin, comprising contacting or treating the body surface with a composition of the present invention.

The present invention also relates to compositions of the present invention that may be used as consumer products or articles selected from the group consisting of a fabric care product, an air care product, or a home care product. Therefore, according to this embodiment, the present invention provides a method of modifying or enhancing the odour properties of a substrate, preferably fabric, furnishings, dishes, hard surfaces and related materials, comprising contacting or treating the substrate with a composition of the present invention.

In another aspect, the present invention is directed to a method of enhancing the fragrance profile of a composition, preferably by improving the longevity of a character of the composition. The method comprises bringing into contact or mixing at least one substantially non-odorous fragrance fixative with the fragrance material according to the composition of the present invention. Preferably, the character is derived from the volatile fragrance materials in the composition and is characterized by a floral character or aromatic/spicy character. Non-limiting examples of floral character include: lavender-type note, a rose-type note, a lily of the valley-type note, a muguet-type note, a jasmine-type note, a magnolia-type note, a cyclamen-type note, a hyacinth-type note, a lilac-type note, an orange blossom-type note, a cherry blossom-type note, a peony-type note, a lotus-type note, a linden blossom-type note, an osmanthus-type note, a heliotrope-type note, a violet-type note, an orris-type note, a tiare-type, a patchouli-type note and the like.

Non-limiting examples of aromatic (or haerbaceous) and spicy character include: cinnamon, cloves, coriander, ginger, saffron, peppers of various kinds (e.g.: black pepper, pink pepper), caraway, cardamom, anise, tea, coffee, cumin, nutmeg, coumarin, basil, rosemary, thyme, mint, tarragon, marjoram, fennel, sage, and juniper.

Preferably, the fragrance profile or character of the composition of the present invention is detectable by a consumer at later time points such as, for example, 1 hour, 2 hours, 3 hours, 4 hours, 5 hours, 6 hours, 7 hours, 8 hours, 10 hours, and possibly all the way up to 24 hours after application of the composition to a substrate as compared to controls.

In another aspect, the present invention is also directed to a method of producing a consumer product comprising bringing into contact or mixing into the product an organoleptically active quantity of a composition of the present invention. Preferably, the present invention is also directed to a perfuming consumer product or article comprising a composition according to the present invention, wherein the perfuming consumer product is selected from the group consisting of a fabric care product, an air care product or a home care product.

### Substantially Non-Odorous Fragrance Fixatives

In one aspect, compositions of the present invention comprise at least one substantially non-odorous fixative selected from the group consisting of the materials disclosed in Table 1.

TABLE 1
Substantially Non-Odorous Fragrance Fixatives

| | | | CAS | |
|---|---|---|---|---|
| No. | Chemical or INCI Name | Trade Name | Number | Supplier |
| 1. | C12-14 Sec-Pareth-3 | Tergitol ® 15-S-7 | 68131- | Sigma Aldrich |
| | | | 40-8 | (UK) |
| 2. | Poly(ethylene glycol-ran- | PPG-7-Buteth-10 | 9038- | Sigma Aldrich |
| | propylene glycol) monobutyl | | 95-3 | (UK) |
| | ether | | | |
| 3. | PPG-4-Ceteth-10 | Nikkol PBC-33 | 37311- | Chemical Navi |
| | | | 01-6 | |
| 4. | Deceth-4 | Ethal DA-4 | 5703- | Ethox |
| | | | 94-6 | Chemicals, |
| | | | | Inc. |
| 5. | PPG-5-Ceteth-20 | AEC PPG-5-Ceteth-20 | 9087- | A & E |
| | | | 53-0 | Connock |
| | | | | (Perfumery & |
| | | | | Cosmetics) |
| | | | | Ltd. |
| 6. | C14-15 Pareth-7 | Neodol 45-7 alcohol | 68951- | Shell |
| | | ethoxylate | 67-7 | Chemical |
| | | | | Company |
| 7. | Linear alcohol (C12-15) | Bio-soft N25-7 | 68131- | Stephan |
| | Pareth-3ethoxylate, POE-7 | | 39-5 | Company |
| 8. | Linear alcohol (C12-13) | Bio-soft N23-6.5 | 66455- | (USA) |
| | Pareth-3ethoxylated, POE-6.5) | | 14-9 | |
| 9. | Polyethylene glycol 1100 | Cremophor ® A 25 | 68439- | Sigma Aldrich |
| | mono(hexadecyl/octadecyl) | | 49-6 | (UK) |
| | ether | | | |
| 10. | Linear alcohol (C9-11) | Bio-soft N91-8 | 68439- | Stephan |
| | ethoxylated POE-8 Pareth-3 | | 46-3 | Company (USA) |
| 11. | Coceth-10 or Polyoxyethylene | Genapol ® C-100 | 61791-13-7 | Sigma Aldrich (UK) |
| | (10) dodecyl ether | | | |
| 12. | Alcohols, C12-14, ethoxylated | Rhodasurf ® LA 30 | 68439-50-9 | Solvay |
| | | | | Solutions Italia S.p.A |
| 13. | Poly(ethylene glycol) | Poly(ethylene glycol) | 9004-74-4 | Sigma Aldrich (UK) |
| | methyl ether | methyl ether | | |
| 14. | C10-16 Pareth-1 | Neodol ® PC 110 | 68002-97-1 | Shell Chemical |
| | | | | Company |
| 15. | PPG-11 Stearyl Ether Kj | Arlamol ™ PS11E | 25231-21-4 | Croda (UK) |
| 16. | Steareth-100 | Brij ® S100 | 9005-00-9 | Sigma Aldrich (UK) |
| 17. | Polyethylene glycol | Brij ® C-58 | 9004-95-9 | Sigma Aldrich (UK) |
| | hexadecyl ether | | | |
| 18. | Pluronic ® F-127 | Pluronic ® F-127 | 9003-11-6 | Sigma Aldrich (UK) |
| 19. | Linear Alcohol (C11) | Bio-soft N1-5 | 34398-01-1 | Stepan Canada Inc. |
| | Elhoxylate, POE-5 | | | |
| 20. | Laureth-10 | Intrasol FA 12/18/10 | 6540-99-4 | Evonik Industries AG |
| 21. | Decaethylene glycol | Polyoxyethylene (10) | 9002-92-0 | Sigma Aldrich (UK) |
| | mono-dodecyl ether | lauryl ether | | |
| 22. | Ethylene glycol | 2-Methoxyethanol | 109-86-4 | Sigma Aldrich (UK) |
| | monomethyl ether | | | |
| 23. | Myreth-4 | Homulgator 920 G | 27306-79-2 | Grau Aromatics |
| | | | | GmbH & Company KG |
| 24. | Oleth-16 Alkoxylated Alcohols | Pegnol O-16A | 25190-05-0 | Toho Chemical |
| | | | | Industry Co., Ltd. |
| 25. | Isosteareth-5 | Emalex 1805 | 52292-17-8 | Nihon Emulsion |
| | | | | Company, Ltd. |
| 26. | PPG-10 Cetyl Ether | Arlamol ™ PC 10 | 9035-85-2 | Croda (UK) |
| 27. | Polyoxy(ethylene glycol) | Poly(ethylene glycol) | 24938-91-8 | Sigma-Aldrich (UK) |
| | (18) tridecyl ether | (18) tridecyl ether | | |
| 28. | Poly(oxy-1,2-ethanediyl), | ALFONIC ® 10-8 | 26183-52-8 | Sasol Chemicals |
| | a-decyl-w-hydroxy- | Ethoxylate | | (USA) LLC |
| 29. | Laureth-1 | Mackam ™ 2LSF | 4536-30-5 | Rhodia (DE) |
| 30. | PEG-5 Hydrogenated | Ethox HTAM-5 | 61791-26-2 | Ethox Chemicals, Inc. |
| | Tallow Amine | | | |
| 31. | PEG-15 Oleamine | Nikkol TAMNO-15 | 26635-93-8 | Nikko Chemicals |
| | | | | Co., Ltd. |
| 32. | Polyoxyethylene (20) oleyl ether | Brij ® O20-SS | 9004-98-2 | Sigma Aldrich (UK) |
| 33. | Cetoleth-10 | Brij ® CO10 | 8065-81-4 | Croda, Inc. |
| 34. | Talloweth-7 | Emulmin 70 | 61791-28-4 | Sanyo Chemical |
| | | | | Industries Ltd. |
| 35. | Isobutoxypropanol Alcohols | Isobutoxypropanol | 34150-35-1 | MolPort |
| 36. | Isobutoxypropanol Alcohols | Isobutoxypropanol | 23436-19-3 | AKos Consulting & |
| | | | | Solutions |
| 37. | Dielhylene Glycol | Twincide EDG | 111-46-6 | Roda |
| 38. | Methoxyethanol | Hisolve MC | 109-86-4 | Toho Chemical |
| | | | | Industry Co., Ltd. |
| 39. | Ethoxyethanol Alcohols | 2-Ethoxyethanol | 110-80-5 | Sigma-Aldrich (UK) |
| 40. | Methoxyisopropanol Alcohols | Dowanol ™ PM | 107-98-2 | The Dow Chemical |
| | | | | Company |
| 41. | Methoxyethanol | Hisolve MC | 32718-54-0 | Toho Chemical |
| | | | | Industry Co., Ltd. |
| 42. | Methylal Ethers | Dimethoxymelhane | 109-87-5 | Sigma-Aldrich (UK) |
| 43. | 3-Methoxybutanol | Methoxybutanol | 2517-43-3 | Hans Schwarzkopf |
| | | | | GmbH/Co. KG |
| 44. | Butoxyethanol | Butyl OXITOL | 111-76-2 | Shell Chemical |
| | | | | Company |
| 45. | Propylene Glycol n-Butyl Ether | Dowanol ™ PnB | 5131-66-8/ | The Dow Chemical |
| | | | 29387-86-8 | Company |
| 46. | Propylene Glycol Butyl Ether | Propylene Glycol Butyl Ether | 15821-83-7 | Sigma Aldrich (UK) |
| 47. | 2-(2-butoxyethoxy)ethanol | Diethylene glycol butyl ether | 112-34-5 | Sigma Aldrich (UK) |
| 48. | Deceth-4 Phosphate | Crodafos ™ D4A | 52019-36-0 | Croda, Inc. |
| 49. | 2-(Hexadecyloxy)ethanol | Ethylene glycol | 2136-71-2 | Sigma-Aldrich (UK) |
| | | monohexadecyl ether | | |
| 50. | Poly(propylene glycol) | Poly(propylene glycol) | 9003-13-8 | Sigma-Aldrich (UK) |
| | monobutyl ether | monobutyl ether | | |
| 51. | Propylene Glycol Propyl Ether | Dowanol ™ PnP | 30136-13-1 | The Dow Chemical |
| | | | | Company |
| 52. | Propylene Glycol n-Butyl Ether | Dowanol ™ PnB | 29387-86-8/ | The Dow Chemical |
| | | | 5131-66-8 | Company |
| 53. | Dipropylene glycol | Di(propylene glycol) methyl | 34590-94-8 | Sigma Aldrich (UK) |
| | monomethyl ether | ether, mixture of isomers | | |
| 54. | Dipropylene Glycol | Proglyde ™ DMM | 111109-77-4 | The Dow Chemical |
| | Dimethyl Ether | | | Company |
| 55. | PPG-2 Methyl Ether | Dowanol ™ DPM | 13429-07-7 | The Dow Chemical |
| | | | | Company |
| 56. | Methoxydiglycol Ethers | OriStar DEGME | 111-77-3 | Orient Stars LLC |
| 57. | Diethylene glycol ethyl ether | Di(ethylene glycol) ethyl ether | 111-90-0 | Sigma Aldrich (UK) |
| 58. | Dimethoxydiglycol Ethers | Dimethyldiglycol | 111-96- | H&V Chemicals |
| 59. | PPG-3 Methyl Ether | Dowanol ™ TPM | 37286-64-9 | The Dow Chemical |
| | | | | Company |
| 60. | Methyl Morpholine | 224286 ALDRICH | 7529-22-8 | Sigma-Aldrich (UK) |
| | Oxide Amine Oxides | 4-Methylmorpholine N-oxide | | |
| 61. | Oleth-3 | Brij ® O3 | 5274-66-8 | Croda Europe, Ltd. |
| 62. | Tri(propylene glycol) | Dowanol ™ TPnB | 55934-93-5 | Sigma-Aldrich (UK) |
| | n-butyl ether | | | |
| 63. | Tripropylene Glycol | Tripropylene Glycol | 24800-44-0 | Sigma-Aldrich (UK) |
| 64. | PPG-3 Methyl Ether | Dowanol ™ TPM | 25498- | The Dow |
| | Alkoxylaled Alcohols | | 49-1 | Chemical Company |
| 65. | Triethylene glycol | Triglycol | 112-27-6 | Sigma Aldrich (UK) |
| 66. | PEG-3 Methyl Ether | Hymol ™ | 112-35-6 | Toho Chemical |
| | | | | Industry Co., Ltd. |
| 67. | Laureth-3 | AEC Laureth-3 | 3055-94-5 | A & E Connock |
| | | | | (Perfumery & |
| | | | | Cosmetics) Ltd. |
| 68. | Ethylhexylglycerin | AG-G-75008 | 70445-33-9 | Angene Chemical |
| 69. | Tetra(ethylene glycol) | Tetraethylene glycol | 112-60-7 | Sigma Aldrich (UK) |
| 70. | Steareth-3 | Isoxal 5 | 4439-32-1 | Vevy Europe SpA |
| 71. | Ceteth-3 | Emalex 103 | 4484-59-7 | Nihon Emulsion |
| | | | | Company, Ltd. |
| 72. | Myreth-3 | Isoxal 5 | 26826-30-2 | Vevy Europe SpA |
| 73. | Trideceth-3 | Alfonic ® TDA- | — | Sasol North America. |
| | | 3 Ethoxylate | | Inc. |
| 74. | Ceteth-2 | Brij ® C2 | 5274-61-3 | Croda Europe, Ltd. |
| 75. | Oleth-2 | Brij ® O2 | 5274-65-7 | Croda, Inc. |
| 76. | Steareth-2 | Brij ® S2 | 16057-43-5 | Croda, Inc. |
| 77. | Cetoleth-10 | Brij ® CO10 | 8065-81-4 | Croda, Inc. |
| 78. | Trimethyl Pentanol | Trimethyl Pentanol | 68959-25-1 | Angene Chemical |
| | Hydroxyethyl Ether Alcohols | Hydroxyethyl Ether | | |
| 79. | Steareth-10 Allyl Ether | Salcare ® SC80 | 109292-17-3 | BASF |
| 80. | TEA-Lauryl Ether | material ID-AG-J-99109 | 1733-93-3 | Angene Chemical |
| 81. | Polyglyceryl-2 Oleyl Ether | Chimexane NB | 71032-90-1 | Chimex |
| 82. | Batyl Alcohol | B402 ALDRICH | 544-62-7 | Sigma-Aldrich (UK) |
| 83. | Octaethylene Glycol | 15879 ALDRICH | 5117-19-1 | Sigma-Aldrich (UK) |
| 84. | Triglycerol diisostearate | Cithrol ™ | 66082-42-6 | Croda (UK) |
| 85. | Diglycerin | Diglycerin 801 | 59113-36-9 | Sakamoto Yakuhin |
| | | | | Kogyo Co., Ltd. |
| 86. | Polyglycerin #310 | Polyglycerin #310 | 25618-55-7 | Sakamoto Yakuhin |
| | | | | Kogyo Co., Ltd. |
| 87. | Distearyl Ether | Cosmacol ® SE | 6297-03-6 | Sasol Germany GmbH |
| 88. | Caprylyl Glyceryl Ether | Caprylyl Glyceryl Ether | 10438-94-5 | AKos Consulting & |
| | | | | Solutions |
| 89. | Chimyl Alcohol | Chimyl Alcohol | 506-03-6 | Nikko Chemicals |
| | | | | Co., Ltd. |
| 90. | Dipentaerythrityl | Liponate ® DPC-6 | 68130-24-5 | Lipo Chemicals, Inc. |
| | Hexacaprylate/Hexacaprate | | | |
| 91. | Morpholine | 394467 ALDRICH | 110-91-8 | Sigma-Aldrich (UK) |
| 92. | Dimethyl Oxazolidine | OXABAN ™-A | 51200-87-4 | The Dow Chemical |
| | | | | Company |
| 93. | Ethyl Hydroxymethyl | 4-Oxazolemethanol | 68140-98-7 | Angene Chemical |
| | Oleyl Oxazoline | | | |
| 94. | Methyl Hydroxymethyl | Adeka Nol GE-RF | 14408-42-5 | Adeka Corporation |
| | Oleyl Oxazoline | | | |
| 95. | Pramoxine HCl | OriStar PMHCL | 637-58-1 | Orient Stars LLC |
| 96. | Allantoin Ascorbate | Allantoin Ascorbate | 57448-83-6 | ABI Chem |
| 97. | Stearamidopropyl | Mackalene ™ 326 | 55852-14-7 | Rhodia Inc. |
| | Morpholine Lactate | | | |
| 98. | Dioxolane | Elcotal DX | 646-06-0 | Lambiotte & CIE S.A. |
| 99. | Glycerol Formal | Glycerol Formal | 5464-28-8 | Sigma Aldrich (UK) |
| 100. | Stearamidopropyl Morpholine | Mackine 321 | 55852-13-6 | Rhodia Inc. |
| 101. | 2,4,6-Tris[bis(methoxymethyl) | Poly(melamine-co- | 68002-20-0 | Sigma-Aldrich (UK) |
| | amino]-1,3,5-triazine | formaldehyde)methylated | | |
| 102. | Poloxamine 1307 | Pluracare ® 1307 | 11111-34-5 | BASF |
| 103. | Nonoxynol-8 | Igepal ® CO-610 | 27177-05-5 | Rhodia Inc. |
| 104. | Nonoxynol-10 | Igepal ® CO-710 | 27177- | Rhodia Inc. |
| | | | 08-8 | |
| 105. | Octoxynol-10 | Nikkol OP-10 | 2315-66-4 | Nikko Chemicals |
| | | | | Co., Ltd. |
| 106. | Nonoxynol-9 | Igepal ® CO-630 | 68987-90-6 | Rhodia Inc. |
| 107. | Nonoxynol-9 Iodine | Nonoxynol-9 iodine | 94349-40-3 | Angene Chemical |
| 108. | Octylphenoxy | Igepal ® CA-630 | 68987-90-6 | Rhodia Inc. |
| | poly(ethyleneoxy)ethanol, | | | |
| | branched | | | |
| 109. | Sodium Octoxynol-2 | Triton™ X-200 | 55837-16-6 | The Dow Chemical |
| | Ethane Sulfonate | | | Company |
| 110. | Benzylhemiformal | Preventol D2 | 14548-60-8 | Lanxess Corporation |
| 111. | Nonoxynol-2 | Igepal ® CO-210 | 27176-93-8 | Rhodia Inc. |
| 112. | Octoxynol-3 | Igepal ® CA-420 | 2315-62-0 | The Dow Chemical |
| | | | | Company |
| 113. | Nonoxynol-3 | Marlophen NP 3 | 27176-95-0 | Sasol Germany GmbH |
| 114. | Alkoxylaled Alcohols | Alkasurf NP-4 | 7311-27-5 | Rhodia Inc. |
| 115. | Nonoxynol-3 | Triethylene Glycol | 51437-95-7 | Santa Cmz |
| | | Mono(p-nonylphenyl)Ether | | Biotechnology |
| 116. | Nonoxynol-7 | Lowenol 2689 | 27177-03-3 | Jos. H. Lowenstein |
| | | | | & Sons, Inc. |
| 117. | Nonoxynol-6 | Igepal ® CO-530 | 27177-01-1 | Rhodia Inc. |
| 118. | Nonoxynol-5 | Igepal ® CO-520 | 20636-48-0 | Rhodia Inc. |
| 119. | Nonoxynol-5 | Igepal ® CO-520 | 26264-02-8 | Rhodia Inc. |
| 120. | Nonoxynol-4 | Alkasurf NP-4 | 27176-97-2 | Rhodia Inc. |
| 121. | Polyglyceryl-10 Trioleate | Nikkol Decaglyn 3-OV | 102051-00-3 | Nikko Chemicals |
| | | | | Co., Ltd. |
| 122. | Polyglyceryl-10 Dioleate | Nikkol Decaglyn 2-O | 33940-99-7 | Nikko Chemicals |
| | | | | Co., Ltd |
| 123. | Polyglyceryl-10 Tetraoleale | Caprol 10G40 | 34424-98-1 | Abitec Corporation |
| 124. | Polyglyceryl-10 Stearate | Nikkol Decaglyn 1-SV | 79777- | Nikko |
| | | EX | 30-3 | Chemicals Co., Ltd. |
| 125. | Polyglyceryl-10 Oleate | S-Face O-1001 P | 79665-93-3 | Sakamoto Yakuhin |
| | | | | Kogyo Co., Ltd. |
| 126. | Polyglyceryl-10 Myristale | Nikkol Decaglyn 1-MV EX | 87390-32-7 | Nikko Chemicals |
| | | | | Co., Ltd. |
| 127. | Dermofeel® G 10 L | Dermofeel ® G 10 L | 34406-66-1 | Dr. Straetnians |
| 128. | Polyglyceryl-6 Laurate | NIKKOL Hexaglyn 1-L | 51033-38-6 | Chemical Navi |
| 129. | Polyglyceryl-6 Isostearate | S-Face IS-601 P | 126928-07-2 | Sakamoto Yakuhin |
| | | | | Kogyo Co., Ltd. |
| 130. | Choleth-10 | Emalex CS-10 | 27321-96-6 | Nihon Emulsion |
| | | | | Company, Ltd. |
| 131. | Steareth-10 Allyl | Salcare ® SC80 | 109292-17-3 | BASF |
| | Ether/Acrylates Copolymer | | | |
| 132. | Polyvinyl Stearyl Ether | Giovarez ® 1800 | 9003-90-7 | Phoenix Chemical, Inc. |
| 133. | Dicetyl Ether | Cosmacol Ether 16 | — | Sasol Germany GmbH |
| 134. | PPG-23-Steareth-34 | Unisafe 34S-23 | 9038-43-1 | Pola Chemical |
| | | | | Industries, Inc. |
| 135. | Stearoxypropyl | Farmin DM E-80 | 17517-01-0 | Kao Corp. |
| | Dimethylamine | | | |
| 136. | Dislearyl Ether | Cosmacol SE | 6297-03-6 | Sasol Germany GmbH |
| 137. | Polyquaternium-10 | AEC Polyquaternium-10 | 55353-19-0 | A & E Connock |
| | | | | (Perfumery & |
| | | | | Cosmetics) Ltd. |
| 138. | Octyl ether | Dioctyl ether | 629-82-3 | Sigma Adlrich (UK) |
| 139. | Ethyl Ether | Diethyl Ether | 60-29-7 | EMD Chemicals |
| 140. | Methyl Hexyl Ether Ethers | methyl hexyl ether | 4747-07-3 | TCI AMERICA |
| 141. | Ceteth-12 | Emalex 112 | 94159-75-8 | Nihon Emulsion |
| | | | | Company, Ltd. |
| 142. | Ceteth-10 or cetyl | Jeecol CA-10 | 14529- | Jeen |
| | alcohol POE-10 | | 40-9 | International |
| 143. | Steareth-10 | Jeecol SA-10 | 13149-86-5 | Jeen International |
| 144. | Nonaethylene glycol | Nonaethylene glycol | 3055-99-0 | Sigma Aldrich (UK) |
| | monododecyl ether | monododecyl ether | | |
| 145. | Oleth-10 | Brij ® O10 | 71976-00-6 | Croda, Inc. |
| 146. | Oleth-10 | Brij ® O10 | 24871-34-9 | Croda, Inc. |
| 147. | PEG-12 | Carbowax ™ PEG 600 | 6790-09-6 | The Dow Chemical |
| | | | | Company |
| 148. | PEG-9 | Sabopeg 400 | 3386-18-3 | Sabo s.p.a. |
| 149. | PEG-10 | DECAETHYLENE | 5579-66-8 | MolPort |
| | | GLYCOL | | |
| 150. | PEG-6 | Carbowax ™ PEG 300 | 2615-15-8 | The Dow Chemical |
| | | | | Company |
| 151. | Glycerol propoxylate | Glycerol propoxylate | 25791-96-2 | Sigma Aldrich (UK) |
| 152. | Glycerol ethoxylate | Glycerol ethoxylate | 31694-55-0 | Sigma Aldrich (UK) |
| 153. | Laureth-8 | AEC Laureth-8 | 3055-98-9 | A & E Con nock |
| | | | | (Perfumery & |
| | | | | Cosmetics) Ltd. |
| 154. | Oleth-8 | Emalex 508 | 27040-03-5 | Nihon Emulsion |
| | | | | Company, Ltd. |
| 155. | Laureth-7 | Alfonic 1216CO-7 | 3055-97-8 | Sasol North |
| | | Ethoxylate | | America, Inc. |
| 156. | Steareth-7 | Polyoxyethylene (7) | 66146-84-7 | Sigma Aldrich |
| | | stearyl ether | | |
| 157. | Deceth-6 | Alfonic 1012-6.0 Ethoxylate | 5168-89-8 | Sasol North |
| | | | | America, Inc. |
| 158. | Steareth-6 | Emalex 606 | 2420-29-3 | Nihon Emulsion |
| | | | | Company, Ltd. |
| 159. | Hexaethylene | Hexaethylene glycol | 3055-96-7 | Sigma-Aldrich (UK) |
| | glycol monododecyl ether | monododecyl ether | | |
| 160. | Hexaethylene glycol | Hexaethylene glycol | 5168-91-2 | Sigma-Aldrich (UK) |
| | monohexadecyl ether | monohexadecyl ether | | |
| 161. | Beheneth-5 | Nikkol BB-5 | 136207-49-3 | Nikko Chemicals |
| | | | | Co., Ltd. |
| 162. | Myreth-5 | Isoxal 12 | 92669-01-7 | Vevy Europe SpA |
| 163. | Steareth-5 | Jeecol SA-5 | 71093-13-5 | Jeen International |
| | | | | Corporation |
| 164. | Ceteth-5 | Emalex 105 | 4478-97-1 | Nihon Emulsion |
| | | | | Company, Ltd. |
| 165. | Oleth-5 | Brij ® O5 | 5353-27-5 | Croda, Inc. |
| 166. | Laureth-5 | Safol ® 23E5 Ethoxylate | 3055-95-6 | Sasol North |
| | | | | America, Inc. |
| 167. | Steareth-4 | Jeecol SA-4 | 59970-10-4 | Jeen International |
| | | | | Corporation |
| 168. | Laureth-4 | Brij ® L4 | 5274-68-0 | Croda, Inc. |
| 169. | Myreth-4 | Homulgator 920 G | 39034-24-7 | Grau Aromatics GmbH |
| | | | | & Company KG |
| 170. | Ceteth-4 | Procol CA-4 | 5274-63-5 | Protameen Chemicals |
| 171. | Oleth-4 | Chemal OA-4 | 5353-26-4 | Chemax, Inc. |
| 172. | Oleth-4 | Chemal OA-4 | 103622-85-1 | Chemax, Inc. |
| 173. | Polyimide-1 | Aquaflex ™ XL-30 | 497926-97-3 | Chemwill |
| 174. | Polymethoxy Bicyclic | Caswell No. 494CA | 56709-13-8 | Angene Chemical |
| | Oxazolidine | | | |
| 175. | Hydroxymethyl | Zoldine ™ ZT | 6542-37-6 | Angus Chemical |
| | Dioxoazabicyclooctane | | | Company |
| 176. | Dihydro-7a-ethyloxazolo | 5-Ethyl-1-aza-3,7- | 7747-35-5 | Sigma Aldrich (UK) |
| | [3,4-c]oxazole | dioxabicyclo[3.3.0]octane | | |
| 177. | Dibenzylidene Sorbitol | Disorbene ® | 32647-67-9 | Roquette America, Inc. |
| 178. | Dimethyldibenzylidene | Millad ® 3988 | 135861-56-2 | Milliken Chemicals |
| | Sorbitol | | | |
| 179. | Laureth-2 | Alfonic 1216CO-2 | 3055-93-4 | Sasol North |
| | | Ethoxylate | | America, Inc. |
| 180. | 2-(2-Butoxyethoxy)ethyl | Piperonyl Butoxide | 51-03-6 | Sigma-Aldrich (UK) |
| | (6-propylpiperonyl) ether | | | |
| 181. | Menthone Glycerin Acetal | Frescolat ® MGA | 63187-91-7 | Symrise |
| 182. | Propylene Glycol Caprylate | Mackaderm PGC | 68332-79-6 | Rhodia Inc. |
| 183. | Diethoxynonadiene | SBB016951 | 67674-36-6 | Ambi liter |
| 184. | Menthoxypropanediol | Coolact ® 10 | 87061-04-9 | Takasago |
| | Alcohols | | | International |
| | | | | Corporation |
| 185. | 2-Diphenylmethoxy-N,N- | Diphenhydramine HCl | 147-24-0 | Sigma-Aldrich (UK) |
| | dimethylethylamine | | | |
| | hydrochloride | | | |
| 186. | 3-((2-ethylhexyl)oxy) | — | 70445-33-9 | — |
| | propane-1,2-diol | | | |
| 187. | 3-((2-propylheptyl)oxy) | — | — | — |
| | propane-1,2-diol | | | |
| 188. | 1-amino-3-((2-ethylhexyl)oxy) | — | 99509-00-9 | — |
| | propan-2-ol | | | |
| 189. | 1-(1-Methyl-2- | Di(propylene glycol) | 29911-27-1 | Sigma Aldrich (UK) |
| | propoxyelhoxy)-2-propanol | propyl ether | | |
| 190. | propyl {4-[2-(diethylamino)-2- | Kolliphor ® EL | 61791-12-6 | Sigma Aldrich (US) |
| | oxoethoxy]-3-methoxyphenyl) | | | |
| | acetate | | | |
| 191. | Bis-methoxy PEG-13 | Expert Gel ® EG56 | 936645-35-1 | PolymerExpert S.A. |
| | PEG-438/PPG-110 SMDI | | | (Pessac, France) |
| | Copolymer | | | |

The compounds selected from the group consisting of Table 1 substantially non-odorous fragrance fixatives 1-190, 191, and mixtures thereof, act as a substantially non-odorous fragrance fixative of the present invention. For example, the substantially non-odorous fragrance fixatives, with a fragrance component act to prolong the duration during which the fragrance profile, preferably the characters attributable from the volatile fragrance materials, can be perceived as compared to a control composition in the absence of the fixatives. As another example, the substantially non-odorous fragrance fixatives with a fragrance component, can improve the fidelity of the fragrance profile, preferably the fragrance component derived from the volatile fragrance materials, such that it remains significantly the same from initial impression to the end as compared to a control composition in the absence of the substantially non-odorous fragrance fixatives. While not wishing to be bound by theory, it is believed that the substantially non-odorous fragrance fixatives associate to the fragrance materials and retard evaporation. This may be due to a combination of both the functionality and the structure of the substantially non-odorous fragrance fixatives and the fragrance materials.

### Fragrance Materials

Preferably, the “fragrance materials” have been classified as low volatile fragrance materials or volatile fragrance materials by their vapor pressure. For the purpose of clarity, when the fragrance materials refer to a single individual compound, its vapor pressure should be determined according to the reference program cited above. In the case that the fragrance materials are a natural oil, extract or absolute, which comprises a mixture of several compounds, the vapor pressure of the complete oil should be treated a mixture of the individual perfume raw material components using the reference program cited above. The individual components and their level, in any given natural oil or extract, can be determined by direct injection of the oil into a GC-MS column for analysis as known by one skilled in the art. In the scenario that the fragrance materials are a proprietary specialty accord, so called ‘bases’, the vapor pressure, using the reference program cited above, should preferably be obtained from the supplier. However, it is understood by one skilled in the art that they can physically analyze the composition of a full fragrance oil available commercially to identity the fragrance raw materials and their levels using standard GC-MS techniques. This would be irrespective of whether they had been added to the fragrance oil as individual chemicals, as components of naturals or from proprietary bases. Although proprietary bases and naturals are included in our examples, when analyzing a commercially available fragrance via GC-MS one could simply identify the components of the base or natural oil as part of the overall fragrance mixture and their levels, without being able to identify which proprietary base or natural oil the fragrance had come from.

The nature and type of fragrance materials in the compositions according to the present invention can be selected by the skilled person, on the basis of its general knowledge together with the teachings contained herein, with reference to the intended use or application of the composition and the desired fragrance profile effect. Examples of suitable fragrance materials are disclosed in U.S. Pat. No. 4,145,184, U.S. Pat. No. 4,209,417, U.S. Pat. No. 4,515,705, and U.S. Pat. No. 4,152,272.

(i) Low Volatile Fragrance Materials

Fragrance materials classified as “low volatile fragrance materials” are ones having a vapor pressure less than 0.001 Torr (0.000133 kPa) at 25° C. Preferably, the low volatile fragrance materials form the at most about 30 wt %, wherein the wt % is relative to the total weight of the fragrance component. Preferably, the low volatile fragrance material is selected from at least 1 material, or at least 2 materials, or at least 3 materials, or at least 5 materials, or at least 7 materials as disclosed in Table 2.

TABLE 2
Low Volatile Fragrance Materials

| | CAS | | | Vapor Pressure |
|---|---|---|---|---|
| No. | Number | IUPAC Name | Common Name** | (Torr at 25° C.)* |
| 1. | 1211-29-6 | Cyclopentaneacetic acid, 3-oxo-2- | Methyl jasmonate | 0.00096500 |
| | | (2Z)-2-penten-1-yl-, methyl ester, | | |
| | | (1R,2R)- | | |
| 2. | 28219-60-5 | 2-Buten-1-ol, 2-methyl-4- | Hindinol | 0.00096100 |
| | | (2,2,3-trimethyl-3-cyclopenten-1-yl)- | | |
| 3. | 93-08-3 | Ethanone, 1-(2-naphthalenyl)- | Methyl beta- | 0.00095700 |
| | | | naphthyl ketone | |
| 4. | 67633-95-8 | 3-Decanone, 1-hydroxy- | Methyl Lavender | 0.00095100 |
| | | | Ketone | |
| 5. | 198404-98-7 | Cyclopropanemethanol, 1-methyl-2- | Javanol ® | 0.00090200 |
| | | [(1,2,2-trimethylbicyclo[3.1.0] | | |
| | | hex-3-yl)methyl]- | | |
| 6. | 121-32-4 | Benzaldehyde, 3-ethoxy-4-hydroxy- | Ethyl vanillin | 0.00088400 |
| 7. | 72403-67-9 | 3-Cyclohexene-1-methanol, 4- | Myraldylacetate | 0.00087900 |
| | | (4-methyl-3-penten-1-yl)-, 1-acetate | | |
| 8. | 28940-11-6 | 2H-1,5-Benzodioxepin-3(4H)- | Calone | 0.00083100 |
| | | one, 7-methyl- | | |
| 9. | 139504-68-0 | 2-Butanol, 1-[[2-(1,1-dimethylethyl) | Amber core | 0.00080300 |
| | | cyclohexyl)oxy]- | | |
| 10. | 502847-01-0 | Spiro[5.5]undec-8-en-1-one, 2,2,7, | Spiro[5.5]undec- | 0.00073100 |
| | | 9-tetramethyl- | 8-en-1-one, 2,2,7, | |
| | | | 9-tetramethyl- | |
| 11. | 2570-03-8 | Cyclopentaneacetic acid, 3-oxo-2-pentyl-, | trans-Hedione | 0.00071000 |
| | | methyl ester, (1R,2R)-rel- | | |
| 12. | 24851-98-7 | Cyclopentaneacetic acid, 3-oxo-2-pentyl-, | Methyl | 0.00071000 |
| | (or 128087-96-7) | methyl ester | dihydrojasmonate | |
| | | | or alternatives ¹ | |
| 13. | 101-86-0 | Octanal, 2-(phenylmethylene)- | Hexyl | 0.00069700 |
| | | | cinnamic aldehyde | |
| 14. | 365411-50-3 | Indeno[4,5-d]-1,3-dioxin, | Nebulone | 0.00069200 |
| | | 4,4a,5,6,7,8,9,9b-octahydro-7,7,8,9,9- | | |
| | | pentamethyl- | | |
| 15. | 37172-53-5 | Cyclopentanecarboxylic acid, | Dihydro Iso | 0.00067500 |
| | | 2-hexyl-3-oxo-, methyl ester | Jasmonate | |
| 16. | 65113-99-7 | 3-Cyclopentene-1-butanol, | Sandalore ® | 0.00062500 |
| | | α,β,2,2,3-pentamethyl- | | |
| 17. | 68133-79-9 | Cyclopentanone, 2-(3,7- | Apritone | 0.00062000 |
| | | dimethyl-2,6-octadien-1-yl)- | | |
| 18. | 7212-44-4 | 1,6,10-Dodecatrien-3-ol, 3,7,11- | Nerolidol | 0.00061600 |
| | | trimethyl- | | |
| 19. | 53243-59-7 | 2-Pentenenitrile, 3-methyl-5- | Citronitril | 0.00061500 |
| | | phenyl-, (2Z)- | | |
| 20. | 134123-93-6 | Benzenepropanenitrile, | Fleuranil | 0.00057600 |
| | | 4-ethyl-α,α-dimethyl- | | |
| 21. | 77-53-2 | 1H-3a,7-Methanoazulen-6-ol, | Cedrol Crude | 0.00056900 |
| | | octahydro-3,6,8,8-tetramethyl-, | | |
| | | (3R,3aS,6R,7R,8aS)- | | |
| 22. | 68155-66-8 | Ethanone, 1-(1,2,3,5,6,7,8,8a- | Iso Gamma Super | 0.00056500 |
| | | octahydro-2,3,8,8-tetramethyl-2- | | |
| | | naphthalenyl)- | | |
| 23. | 54464-57-2 | Ethanone, 1-(1,2,3,4,5,6,7,8- | Iso-E Super ® | 0.00053800 |
| | | tetramethyl-2-naphthalenyl)- | | |
| 24. | 774-55-0 | Ethanone, 1-(5,6,7,8-tetrahydro- | Florantone | 0.00053000 |
| | | 2-naphthalenyl)- | | |
| 25. | 141-92-4 | 2-Octanol, 8,8-dimethoxy-2,6-dimethyl- | Hydroxycitronellal | 0.00052000 |
| | | | Dimethyl Acetal | |
| 26. | 20665-85-4 | Propanoic acid, 2-methyl-, | Vanillin | 0.00051200 |
| | | 4-formyl-2-methoxyphenyl ester | isobutyrate | |
| 27. | 79-78-7 | 1,6-Heptadien-3-one, | Hexalon | 0.00049800 |
| | | 1-(2,6,6-trimethyl-2-cyclohexen-1-yl)- | | |
| 28. | 6259-76-3 | Benzoic acid, 2-hydroxy-, hexyl ester | Hexyl Salicylate | 0.00049100 |
| 29. | 93-99-2 | Benzoic acid, phenyl ester | Phenyl Benzoate | 0.00047900 |
| 30. | 153859-23-5 | Cyclohexanepropanol, 2,2,6- | Norlimbanol | 0.00046900 |
| | | trimethyl-α-propyl-, (1R,6S)- | | |
| 31. | 70788-30-6 | Cyclohexanepropanol, 2,2,6- | Timberol | 0.00046900 |
| | | trimethyl-α-propyl- | | |
| 32. | 68555-58-8 | Benzoic acid, 2-hydroxy-, | Prenyl Salicylate | 0.00045700 |
| | | 3-methyl-2-buten-1-yl ester | | |
| 33. | 950919-28-5 | 2H-1,5-Benzodioxepin-3(4H)- | Cascalone | 0.00045500 |
| | | one, 7-(1-methylethyl)- | | |
| 34. | 30168-23-1 | Butanal, 4-(octahydro-4,7-methano- | Dupical | 0.00044100 |
| | | 5H-inden-5-ylidene)- | | |
| 35. | 1222-05-5 | Cyclopenta[g]-2-benzopyran, | Galaxolide ® | 0.00041400 |
| | | 1,3,4,6,7,8-hexahydro-4,6,6,7,8,8- | | |
| | | hexamethyl- | | |
| 36. | 4602-84-0 | 2,6,10-Dodecatrien-1-ol, 3,7,11- | Farnesol | 0.00037000 |
| | | trimethyl- | | |
| 37. | 95962-14-4 | Cyclopentanone, 2-[2-(4-methyl- | Nectaryl | 0.00036700 |
| | | 3-cyclohexen-1-yl)propyl]- | | |
| 38. | 4674-50-4 | 2(3H)-Naphthalenone, 4,4a,5,6,7,8- | Nootkatone | 0.00035800 |
| | | hexahydro-4,4a-dimethyl-6-(1- | | |
| | | methylethenyl)-, (4R,4aS,6R)- | | |
| 39. | 3487-99-8 | 2-Propenoic acid, 3-phenyl-, pentyl ester | Amyl Cinnamate | 0.00035200 |
| 40. | 10522-41-5 | 2-hydroxy-2-phenylethy acetate | hydroxyphenethyl | 0.00033900 |
| | | | acetate | |
| 41. | 118-71-8 | 4H-Pyran-4-one, 3-hydroxy-2-methyl- | Maltol | 0.00033700 |
| 42. | 128119-70-0 | 1-Propanol, 2-methyl-3-[(1,7,7- | Bornafix | 0.00033400 |
| | | trimethylbicyclo[2.2.1]hept-2-yl) oxy]- | | |
| 43. | 103614-86-4 | 1-Naphthalenol, 1,2,3,4,4a,5,8,8a- | Octalynol | 0.00033200 |
| | | octahydro-2,2,6,8-tetramethyl- | | |
| 44. | 7785-33-3 | 2-Butenoic acid, 2-methyl-, (2E)- | Geranyl Tiglate | 0.00033200 |
| | | 3,7-dimethyl-2,6-octadien-1-yl ester, (2E)- | | |
| 45. | 117933-89-8 | 1,3-Dioxane, 2-(2,4-dimethyl-3-cyclohexen- | Karanal | 0.00033100 |
| | | 1-yl)-5-methyl-5-(1-methylpropyl)- | | |
| 46. | 629-92-5 | Nonadecane | Nonadecane | 0.00032500 |
| 47. | 67801-20-1 | 4-Penten-2-ol, 3-methyl-5-(2,2,3- | Ebanol | 0.00028100 |
| | | trimethyl-3-cyclopenten-1-yl)- | | |
| 48. | 65416-14-0 | Propanoic acid, 2-methyl-, 2-methyl-4- | Maltol | 0.00028000 |
| | | oxo-4H-pyran-3-yl ester | Isobutyrate | |
| 49. | 28219-61-6 | 2-Buten-1-ol, 2-ethyl-4-(2,2,3-trimethyl- | Laevo | 0.00028000 |
| | | 3-cyclopenten-1-yl)- | Trisandol | |
| 50. | 5986-55-0 | 1,6-Methanonaphthalen-1(2H)-ol, octahydro- | Healingwood | 0.00027800 |
| | | 4,8a,9,9-tetramethyl-, (1R,4S,4aS,6R,8aS)- | | |
| 51. | 195251-91-3 | 2H-1,5-Benzodioxepin-3(4H)-one, | Transluzone | 0.00026500 |
| | | 7-(1,1-dimethylethyl)- | | |
| 52. | 3100-36-5 | 8-Cyclohexadecen-1-one | Cyclohexadecenone | 0.00025300 |
| 53. | 65405-77-8 | Benzoic acid, 2-hydroxy-, (3Z)-3- | cis-3-Hexenyl | 0.00024600 |
| | | hexen-1-yl ester | salicylate | |
| 54. | 4940-11-8 | 4H-Pyran-4-one, 2-ethyl-3-hydroxy- | Ethyl Maltol | 0.00022800 |
| 55. | 541-91-3 | Cyclopentadecanone, 3-methyl- | Muskone | 0.00017600 |
| 56. | 118-58-1 | Benzoic acid, 2-hydroxy-, | Benzyl | 0.00017500 |
| | | phenylmethyl ester | salicylate | |
| 57. | 81783-01-9 | 6,8-Nonadien-3-one, 2,4,4,7- | Labienoxime | 0.00017300 |
| | | tetramethyl-, oxime | | |
| 58. | 25485-88-5 | Benzoic acid, 2-hydroxy-, | Cyclohexyl | 0.00017300 |
| | | cyclohexyl ester | Salicylate | |
| 59. | 91-87-2 | Benzene, [2-(dimethoxymethyl)- | Amyl Cinnamic | 0.00016300 |
| | | 1-hepten-1-yl]- | Aldehyde Dimethyl | |
| | | | Acetal | |
| 60. | 104864-90-6 | 3-Cyclopentene-1-butanol, | Firsantol | 0.00016000 |
| | | (β,2,2,3-tetramethyl-δ-methylene- | | |
| 61. | 224031-70-3 | 4-Penten-1-one, 1-spiro[4.5]dec-7- | Spirogalbanone | 0.00015300 |
| | | en-7-yl- | | |
| 62. | 134-28-1 | 5-Azulenemethanol, 1,2,3,4,5,6,7,8- | Guaiyl Acclaic | 0.00013400 |
| | | ociahydro-α,α,3,8-tetramethyl-, 5- | | |
| | | acetate, (3S,5R,8S)- | | |
| 63. | 236391-76-7 | Acetic acid, 2-(1-oxopropoxy)-, 1- | Romandolide ® | 0.00012400 |
| | | (3,3-dimethylcyclohexyl)ethyl ester | | |
| 64. | 115-71-9 | 2-Penten-1-ol, 5-[(1R,3R,6S)-2,3- | cis-alpha- | 0.00011800 |
| | | dimethyltricyclo[2.2.1.02,6]hept- | Santalol | |
| | | 3-yl]-2-methyl-, (2Z)- | | |
| 65. | 107898-54-4 | 4-Penten-2-ol, 3,3-dimethyl-5- | Polysantol ® | 0.00011700 |
| | | (2,2,3-trimethyl-3-cyclopenten-1-yl)- | | |
| 66. | 69486-14-2 | 5,8-Methano-2H-1-benzopyran- | Florex ® | 0.00011000 |
| | | 2-one, 6-ethylideneoctahydro- | | |
| 67. | 84697-09-6 | Heptanal, 2-[(4-methylphenyl) | Acalea | 0.00010100 |
| | | methylene]- | | |
| 68. | 14595-54-1 | 4-Cyclopentadecen-1-one, (Z)- | Exaltenone | 0.00009640 |
| 69. | 32388-55-9 | Ethanone, 1-[(3R,3aR,7R,8aS)- | Vertofix ® | 0.00008490 |
| | | 2,3,4,7,8,8a-hexahydro-3,6,8,8- | | |
| | | telramethyl-1H-3a,7- | | |
| | | methanoazulen-5-yl]- | | |
| 70. | 131812-67-4 | 1,3-Dioxolane, 2,4-dimethyl-2- | Okoumal ® | 0.00007600 |
| | | (5,6,7,8-tetrahydro-5,5,8,8- | | |
| | | tetramethyl-2-naphthalenyl)- | | |
| 71. | 106-02-5 | Oxacyclohexadecan-2-one | Exaltolide ® | 0.00006430 |
| 72. | 141773-73-1 | 1-Propanol, 2-[1-(3,3- | Helvetolide ® | 0.00005790 |
| | | dimethylcyclohexyl)ethoxy]-2- | | |
| | | methyl-, 1-propanoate | | |
| 73. | 63314-79-4 | 5-Cyclopentadecen-1- | Delta | 0.00005650 |
| | | one, 3-methyl- | Muscenone | |
| 74. | 77-42-9 | 2-Penten-1-ol, 2-methyl-5- | cis-beta- | 0.00004810 |
| | | [(1S,2R,4R)-2-methyl-3- | Santalol | |
| | | methylenebicyclo[2.2.1] | | |
| | | hept-2-yl], (2Z)- | | |
| 75. | 362467-67-2 | 2H-1,5-Benzodioxepin-3(4H)- | Azurone | 0.00004770 |
| | | one, 7-(3-methylbutyl)- | | |
| 76. | 28371-99-5 | Ethanone, 1-(2,6,10-trimethyl- | Trimofix O | 0.00004580 |
| | | 2,5,9-cyclododecatrien-1-yl)- | | |
| 77. | 16223-63-5 | 1H-3a,6-Methanoaxulene-3- | Khusimol | 0.00004400 |
| | | methanol, octahydro-7,7- | | |
| | | dimethyl-8-methylene-, | | |
| | | (3S,3aR,6R,8aS)- | | |
| 78. | 10461-98-0 | Benzeneacetonitrile, | Peonile | 0.00004290 |
| | | α-cyclohexylidene- | | |
| 79. | 50607-64-2 | Benzoic acid, 2-[(2- | Mevantraal | 0.00004070 |
| | | methylpentylidene) | | |
| | | amino]-, methyl ester | | |
| 80. | 29895-73-6 | 5-Hydroxy-2-benzyl- | Acetal CD | 0.00004050 |
| | | 1,3-dioxane | | |
| 81. | 94-47-3 | Benzoic acid, 2- | Phenyl Ethyl | 0.00003480 |
| | | phenylethyl ester | Benzoate | |
| 82. | 3100-36-5 | Cyclohexadec-8-en-1-one | Globanone ® | 0.00003310 |
| 83. | 37609-25-9 | 5-Cyclohexadecen-1-One | Ambretone | 0.00003310 |
| 84. | 66072-32-0 | Cyclohexanol, 4- | Iso Bornyl | 0.00003010 |
| | | (1,7,7-trimethylbicyclo | Cyclohexanol | |
| | | [2.2.1]hept-2-yl)- | | |
| 85. | 31906-04-4 | 3-Cyclohexene-1- | Lyral ® | 0.00002940 |
| | | carboxaldehyde, 4-(4- | | |
| | | hydroxy-4-methylpentyl)- | | |
| 86. | 21145-77-7 | Ethanone, 1-(5,6,7,8- | Musk Plus | 0.00002860 |
| | | tetrahydro-3,5,5,6,8,8- | | |
| | | hexamethyl-2-naphthalenyl)- | | |
| 87. | 21145-77-7 | Ethanone, 1-(5,6,7,8- | Fixolide | 0.00002860 |
| | | tetrahydro-3,5,5,6,8,8- | | |
| | | hexamethyl-2-naphthalenyl)- | | |
| 88. | 22442-01-0 | 2-Cyclopentadecen- | Museenone | 0.00002770 |
| | | 1-one, 3-methyl- | | |
| 89. | 109-29-5 | Oxacycloheptadecan-2-one | Silvanone Ci | 0.00002600 |
| 90. | 101-94-0 | Benzeneacetic acid, | Para Cresyl | 0.00002330 |
| | | 4-methylphenyl ester | Phenyl Acetate | |
| 91. | 102-20-5 | Benzeneacetic acid, | Phenyl Ethyl | 0.00002300 |
| | | 2-phenylethyl ester | Phenyl Acetate | |
| 92. | 118562-73-5 | Cyclododecaneethanol, | Hydroxyambran | 0.00001800 |
| | | β-methyl- | | |
| 93. | 103-41-3 | 2-Propenoic acid, | Benzyl | 0.00001050 |
| | | 3-phenyl-, phenyl methyl ester | Cinnamate | |
| 94. | 4707-47-5 | Benzoic acid, 2,4-dihydroxy- | Veramoss | 0.00001050 |
| | | 3,6-dimethyl-, methyl ester | | |
| 95. | 183551-83-9 | Naphtho[2,1-b]furan-6(7H)- | Myrrhone | 0.00000977 |
| | | one, 8,9-dihydro-1,5,8- | | |
| | | trimethyl-, (8R)- | | |
| 96. | 102-17-0 | Benzeneacetic acid, (4- | Para Anisyl | 0.00000813 |
| | | methoxyphenyl)methyl ester | Phenyl Acetate | |
| 97. | 120-11-6 | Benzene, 2-methoxy-1- | Benzyl Iso | 0.00000676 |
| | | (phenylmethoxy)-4-(1-propen-1-yl)- | Eugenol | |
| 98. | 102-22-7 | Benzeneacetic acid, (2E)- | Geranyl | 0.00000645 |
| | | 3,7-dimethyl-2,6-octadien-1-yl ester | Phenylacetate | |
| 99. | 111879-80-2 | Oxacyclohexadec-12-en-2- | Habanolide | 0.00000431 |
| | | one, (12E)- | 100% | |
| 100. | 87-22-9 | Benzoic acid, 2-hydroxy-, | Phenyl Ethyl | 0.00000299 |
| | | 2-phenylethyl ester | Salicylate | |
| 101. | 78-37-5 | 2-Propenoic acid, 3-phenyl-, | Linalyl | 0.00000174 |
| | | 1-ethenyl-1,5-dimethyl- | Cinnamate | |
| | | 4-hexen-1-yl ester | | |
| 102. | 28645-51-4 | Oxacycloheptadec-10-en-2-one | Ambrettolide | 0.00000139 |
| 103. | 123-69-3 | Oxacycloheptadec-8-en-2-one, | Ambrettolide | 0.00000136 |
| | | (8Z)- | | |
| 104. | 3391-83-1 | 1,7-Dioxacycloheptadecan- | Musk RI | 0.00000057 |
| | | 8-one | | |
| 105. | 68527-79-7 | 7-Oclen-2-ol, 8-(1H-indol- | Indolene | 0.000000445 |
| | | 1-yl)-2,6-dimethyl- | | |
| 106. | 89-43-0 | Methyl 2-[(7-hydroxy-3,7- | Aurantinol | 0.0000000100 |
| | | dimethyloctylidene)amino]benzoate | | |
| 107. | 54982-83-1 | 1,4-Dioxacyclohexadecane-5,16-dione | Zenolide | 0.00000000834 |
| 108. | 105-95-3 | 1,4-Dioxacycloheptadecane-5,17-dione | Ethylene | 0.00000000313 |
| | | | Brassylate | |
| 109. | 3681-73-0 | Hexadecanoic acid, (2E)-3,7- | Hexarose | 0.00000000300 |
| | | dimethyl-2,6-ocladien-1-yl ester | | |
| 110. | 4159-29-9 | Phenol, 4-[3-(benzoyloxy)-1- | Coniferyl | 0.00000000170 |
| | | propen-1-yl]-2-methoxy- | benzoate | |
| 111. | 144761-91-1 | Benzoic acid, 2-[(1-hydroxy- | Trifone | 0.00000000093 |
| | | 3-phenylbutyl)amino]-, methyl ester | DIPG | |

¹ Non-limiting examples of alternative qualities from various suppliers can be purchased under the following tradenames: Kharismal ® Super (IFF), Kharismal ® (IFF), Hedione ® (Firmenich), Hedione ® HC (Firmenich), Paradisone (Firmenich), Cepionate (Zenon), Super cepionate (Zenon), Claigeon ® (Zenon).
*Vapor Pressures are acquired as described in the Test Methods Section.
**Origin: The low volatile fragrance materials may be obtained from one or more of the following companies: Firmenich (Geneva, Switzerland), Symrise AG (Holzminden, Germany), Givaudan (Argenleuil, France), IFF (Hazlet, New Jersey), Bedoukian (Danbury, Connecticut), Sigma Aldrich (St. Louis, Missouri), Millennium Speciality Chemicals (Olympia Fields, Illinois), Polarone International (Jersey City, New Jersey), and Aroma & Flavor Specialities (Danbury, Connecticut).

(ii) Volatile Fragrance Materials

Fragrance materials classified as “volatile fragrance materials” are ones having a vapor pressure greater than or equal to 0.001 Torr (0.000133 kPa) at 25° C. Preferably, the volatile fragrance materials is present in an amount of from about 70 wt % to about 99.9 wt %, preferably greater than about 80 wt %, or more preferably greater than about 88 wt %, relative to the total weight of the fragrance component. Preferably, the volatile fragrance material is selected from at least 1 material, or at least 2 materials, or at least 3 materials, or at least 5 materials, or at least 7 materials as disclosed in Table 3.

TABLE 3
Volatile Fragrance Materials

| | CAS | | | Vapor Pressure |
|---|---|---|---|---|
| No. | Number | IUPAC Name | Common Name** | (Torr at 25° C.)* |
| 1. | 107-31-3 | Formic acid, methyl ester | Methyl Formate | 732.00000000 |
| 2. | 75-18-3 | Methane, 1,1′-thiobis- | Dimethyl Sulfide | 647.00000000 |
| | | | 1.0% In DEP | |
| 3. | 141-78-6 | Acetic acid ethyl ester | Ethyl Acetate | 112.00000000 |
| 4. | 105-37-3 | Propanoic acid, ethyl | Ethyl Propionate | 44.50000000 |
| | | ester | | |
| 5. | 110-19-0 | Acetic acid, 2- | Isobutyl Acetate | 18.00000000 |
| | | methylpropyl ester | | |
| 6. | 105-54-4 | Butanoic acid, | Ethyl Butyrate | 13.90000000 |
| | | ethyl ester | | |
| 7. | 14765-30-1 | 1-Butanol | Butyl Alcohol | 8.52000000 |
| 8. | 7452-79-1 | Butanoic acid, 2- | Ethyl-2-Methyl | 7.85000000 |
| | | methyl-, ethyl ester | Butyrate | |
| 9. | 123-92-2 | 1-Butanol, 3-methyl-, | Iso Amyl Acetate | 5.68000000 |
| | | 1-acetate | | |
| 10. | 66576-71-4 | Butanoic acid, 2-methyl-, | Iso Propyl 2- | 5.10000000 |
| | | 1-methylethyl ester | Methylbulyrate | |
| 11. | 110-43-0 | 2-Heptanone | Methyl Amyl | 4.73000000 |
| | | | Ketone | |
| 12. | 6728-26-3 | 2-Hexenal, (2E)- | Trans-2 Hexenal | 4.62000000 |
| 13. | 123-51-3 | 1-Butanol, 3-methyl- | Isoamyl Alcohol | 4.16000000 |
| 14. | 1191-16-8 | 2-Buten-1-ol, 3- | Prenyl acetate | 3.99000000 |
| | | methyl-, 1-acetate | | |
| 15. | 57366-77-5 | 1,3-Dioxolane-2- | Methyl Dioxolan | 3.88000000 |
| | | methanamine, N-methyl- | | |
| 16. | 7785-70-8 | Bicyclo[3.1.1]hept-2- | Alpha Pinene | 3.49000000 |
| | | ene, 2,6,6-trimethyl-, (1R,5R)- | | |
| 17. | 79-92-5 | Bicyclo[2.2.1]heptane, | Camphene | 3.38000000 |
| | | 2,2-dimethyl-3-methylene- | | |
| 18. | 94087-83-9 | 2-Butanethiol, | 4-Methoxy-2- | 3.31000000 |
| | | 4-methoxy-2-methyl- | Methyl-2- | |
| | | | Butanenthiol | |
| 19. | 39255-32-8 | Pentanoic acid, | Manzanate | 2.91000000 |
| | | 2-methyl-, ethyl ester | | |
| 20. | 3387-41-5 | Bicyclo[3.1.0]hexane, 4- | Sabinene | 2.63000000 |
| | | methylene-1-(1-methylethyl)- | | |
| 21. | 127-91-3 | Bicyclo[3.1.1]heptane, | Beta | 2.40000000 |
| | | 6-dimethyl-2-methylene- | Pinene | |
| 22. | 105-68-0 | 1-Butanol, 3-methyl-, | Amyl | 2.36000000 |
| | | 1-propanoate | Propionate | |
| 23. | 123-35-3 | 1,6-Octadiene, 7- | Myrcene | 2.29000000 |
| | | methyl-3-methylene- | | |
| 24. | 124-13-0 | Octanal | Octyl Aldehyde | 2.07000000 |
| 25. | 7392-19-0 | 2H-Pyran, 2- | Limetol | 1.90000000 |
| | | ethenyltetrahydro- | | |
| | | 2,6,6-trimethyl- | | |
| 26. | 111-13-7 | 2-Octanone | Methyl Hexyl | 1.72000000 |
| | | | Ketone | |
| 27. | 123-66-0 | Hexanoic acid, | Ethyl | 1.66000000 |
| | | ethyl ester | Caproate | |
| 28. | 470-82-6 | 2-Oxabicyclo[2.2.2] | Eucalyptol | 1.65000000 |
| | | octane, 1,3,3-trimethyl- | | |
| 29. | 99-87-6 | Benzene, 1-methyl-4- | Para Cymene | 1.65000000 |
| | | (1-methylethyl)- | | |
| 30. | 104-93-8 | Benzene, 1-methoxy- | Para Cresyl | 1.65000000 |
| | | 4-methyl- | Methyl Ether | |
| 31. | 13877-91-3 | 1,3,6-Octatriene, | Ocimene | 1.56000000 |
| | | 3,7-dimethyl- | | |
| 32. | 138-86-3 | Cyclohexene, 1-methyl- | dl-Limonene | 1.54000000 |
| | | 4-(1-methylethenyl)- | | |
| 33. | 5989-27-5 | Cyclohexene, 1-methyl- | d-limonene | 1.54000000 |
| | | 4-(1-methylethenyl)-, (4R)- | | |
| 34. | 106-68-3 | 3-Octanone | Ethyl Amyl | 1.50000000 |
| | | | Ketone | |
| 35. | 110-41-8 | Undecanal, 2-methyl- | Methyl Nonyl | 1.43000000 |
| | | | Acetaldehyde | |
| 36. | 142-92-7 | Acetic acid, hexyl esler | Hexyl acetate | 1.39000000 |
| 37. | 110-93-0 | 5-Hepten-2-one, 6-methyl- | Methyl | 1.28000000 |
| | | | Heptenone | |
| 38. | 81925-81-7 | 2-Hepten-4-one, 5-methyl- | Filbertone | 1.25000000 |
| | | | 1% in TEC | |
| 39. | 3681-71-8 | 3-Hexen-1-ol, 1-acetate, (3Z)- | cis-3- | 1.22000000 |
| | | | Hexenyl acetate | |
| 40. | 97-64-3 | Propanoic acid, 2-hydroxy-, | Ethyl | 1.16000000 |
| | | ethyl ester | Lactate | |
| 41. | 586-62-9 | Cyclohexene, 1-methyl- | Terpineolene | 1.13000000 |
| | | 4-(1-methylethylidene)- | | |
| 42. | 51115-64-1 | Butanoic acid, 2- | Amyl butyrate | 1.09000000 |
| | | methylbutyl ester | | |
| 43. | 106-27-4 | Butanoic acid, 3- | Amyl | 1.09000000 |
| | | methylbutyl ester | Butyrate | |
| 44. | 99-85-4 | 1,4-Cyclohexadiene, | Gamma | 1.08000000 |
| | | 1-methyl-4-(1-methylethyl)- | Terpinene | |
| 45. | 18640-74-9 | Thiazole, 2-(2-methylpropyl)- | 2- | 1.07000000 |
| | | | Isobutylthiazole | |
| 46. | 928-96-1 | 3-Hexen-1-ol, (3Z)- | cis-3-Hexenol | 1.04000000 |
| 47. | 100-52-7 | Benzaldehyde | Benzaldehyde | 0.97400000 |
| 48. | 141-97-9 | Butanoic acid, 3-oxo-, | Ethyl | 0.89000000 |
| | | ethyl ester | Acetoacetate | |
| 49. | 928-95-0 | 2-Hexen-1-ol, (2E)- | Trans-2-Hexenol | 0.87300000 |
| 50. | 928-94-9 | 2-Hexen-1-ol, (2Z)- | Beta Gamma | 0.87300000 |
| | | | Hexenol | |
| 51. | 24691-15-4 | Cyclohexane, 3-ethoxy-1, | Herbavert | 0.85200000 |
| | | 1,5-trimethyl-, cis-(9CI) | | |
| 52. | 19872-52-7 | 2-Pentanone, 4- | 4-Methyl-4- | 0.84300000 |
| | | mercapto-4-methyl- | Mercaptopenlan- | |
| | | | 2-one 1 ppm TEC | |
| 53. | 3016-19-1 | 2,4,6-Octatriene, | Allo-Ocimene | 0.81600000 |
| | | 2,6-dimethyl-, (4E,6E)- | | |
| 54. | 69103-20-4 | Oxirane, 2,2-dimethyl- | Myroxide | 0.80600000 |
| | | 3-(3-methyl-2,4-pentadien- | | |
| | | 1-yl)- | | |
| 55. | 189440-77-5 | 4,7-Octadienoic acid, | Anapear | 0.77700000 |
| | | methyl ester, (4E)- | | |
| 56. | 67633-96-9 | Carbonic acid, (3Z)-3- | Liffarome ™ | 0.72100000 |
| | | hexen-1-yl methyl ester | | |
| 57. | 123-68-2 | Hexanoic acid, 2-propen- | Allyl | 0.67800000 |
| | | 1-yl ester | Caproate | |
| 58. | 106-72-9 | 5-Heptenal, 2,6-dimethyl- | Melonal | 0.62200000 |
| 59. | 106-30-9 | Heptanoic acid, ethyl ester | Ethyl | 0.60200000 |
| | | | Oenanthate | |
| 60. | 68039-49-6 | 3-Cyclohexene-1- | Ligustral or | 0.57800000 |
| | | carboxaldehyde, 2,4-dimethyl- | Triplal | |
| 61. | 101-48-4 | Benzene, (2,2- | Phenyl | 0.55600000 |
| | | dimethoxyethyl)- | Acetaldehyde | |
| | | | Dimethyl | |
| | | | Acetal | |
| 62. | 16409-43-1 | 2H-Pyran, tetrahydro-4- | Rose Oxide | 0.55100000 |
| | | methyl-2-(2-methyl-1- | | |
| | | propen-1-yl)- | | |
| 63. | 925-78-0 | 3-Nonanone | Ethyl Hexyl | 0.55100000 |
| | | | Ketone | |
| 64. | 100-47-0 | Benzonitrile | Benzyl Nitrile | 0.52400000 |
| 65. | 589-98-0 | 3-Octanol | Octanol-3 | 0.51200000 |
| 66. | 58430-94-7 | 1-Hexanol, 3,5,5-trimethyl-, | Iso Nonyl | 0.47000000 |
| | | 1-acetate | Acetate | |
| 67. | 10250-45-0 | 4-Heptanol, 2,6-dimethyl-, | Alicate | 0.45400000 |
| | | 4-acetate | | |
| 68. | 105-79-3 | Hexanoic acid, 2- | Iso Butyl | 0.41300000 |
| | | methylpropyl ester | Caproate | |
| 69. | 2349-07-7 | Propanoic acid, 2-methyl-, | Hexyl | 0.41300000 |
| | | hexyl ester | isobulyrate | |
| 70. | 23250-42-2 | Cyclohexanecarboxylic acid, | Cyprissate | 0.40500000 |
| | | 1,4-dimethyl-, methyl | | |
| | | ester, trans- | | |
| 71. | 122-78-1 | Benzeneacetaldehyde | Phenyl | 0.36800000 |
| | | | acetaldehyde | |
| 72. | 5405-41-4 | Butanoic acid, 3-hydroxy-, | Ethyl-3- | 0.36200000 |
| | | ethyl ester | Hydroxy | |
| | | | Butyrate | |
| 73. | 105-53-3 | Propanedioic acid, 1,3- | Diethyl | 0.34400000 |
| | | diethyl ester | Malonate | |
| 74. | 93-58-3 | Benzoic acid, methyl | Methyl | 0.34000000 |
| | | ester | Benzoate | |
| 75. | 16356-11-9 | 1,3,5-Undecatriene | Undecatriene | 0.33600000 |
| 76. | 65405-70-1 | 4-Decenal, (4E)- | Decenal | 0.33100000 |
| | | | (Trans-4) | |
| 77. | 54546-26-8 | 1,3-Dioxane, 2-butyl- | Herboxane | 0.33000000 |
| | | 4,4,6-trimethyl- | | |
| 78. | 13254-34-7 | 2-Heptanol, 2,6-dimethyl- | Dimethyl-2,6- | 0.33000000 |
| | | | Heptan-2-ol | |
| 79. | 98-86-2 | Ethanone, 1-phenyl- | Acetophenone | 0.29900000 |
| 80. | 93-53-8 | Benzeneacetaldehyde, | Hydratropic | 0.29400000 |
| | | α-methyl- | aldehyde | |
| 81. | 80118-06-5 | Propanoic acid, 2-methyl-, | Iso Pentyrate | 0.28500000 |
| | | 1,3-dimethyl-3-buten-1-yl ester | | |
| 82. | 557-48-2 | 2,6-Nonadienal, (2E,6Z)- | EZ-2,6- | 0.28000000 |
| | | | Nonadien-1-al | |
| 83. | 24683-00-9 | Pyrazine, 2-methoxy-3- | 2-Methoxy- | 0.27300000 |
| | | (2-methylpropyl)- | 3-Isobutyl | |
| | | | Pyrazine | |
| 84. | 104-57-4 | Formic acid, phenylmethyl | Benzyl | 0.27300000 |
| | | ester | Formate | |
| 85. | 104-45-0 | Benzene, 1-methoxy-4-propyl- | Dihydroanethole | 0.26600000 |
| 86. | 491-07-6 | Cyclohexanone, 5-methyl-2- | Iso Menthone | 0.25600000 |
| | | (1-methylethyl)-, (2R,5R)-rel- | | |
| 87. | 89-80-5 | Cyclohexanone, 5-methyl-2- | Menthone | 0.25600000 |
| | | (1-methylethyl)-, (2R,5S)-rel- | Racemic | |
| 88. | 2463-53-8 | 2-Nonenal | 2 Nonen-1-al | 0.25600000 |
| 89. | 55739-89-4 | Cyclohexanone, 2-ethyl- | Thuyacetone | 0.25000000 |
| | | 4,4-dimethyl- | | |
| 90. | 150-78-7 | Benzene, 1,4-dimelhoxy- | Hydroquinone | 0.25000000 |
| | | | Dimethyl Ether | |
| 91. | 64988-06-3 | Benzene, 1-(ethoxymethyl)- | Rosacene | 0.24600000 |
| | | 2-methoxy- | | |
| 92. | 76-22-2 | Bicyclo[2.2.1]heptan-2- | Camphor | 0.22500000 |
| | | one, 1,7,7-trimethyl- | gum | |
| 93. | 67674-46-8 | 2-Hexene, 6,6-dimethoxy- | Methyl | 0.21400000 |
| | | 2,5,5-trimethyl- | Pamplemousse | |
| 94. | 112-31-2 | Decanal | Decyl Aldehyde | 0.20700000 |
| 95. | 16251-77-7 | Benzenepropanal, | Trifernal | 0.20600000 |
| | | β-methyl- | | |
| 96. | 93-92-5 | Benzenemethanol, | Methyl- | 0.20300000 |
| | | α-methyl-, 1-acetate | phenylcarbinol | |
| | | | Acetate | |
| 97. | 143-13-5 | Acetic acid, nonyl ester | Nonyl Acetate | 0.19700000 |
| 98. | 122-00-9 | Ethanone, 1-(4- | Para Methyl | 0.18700000 |
| | | methylphenyl)- | Acetophenone | |
| 99. | 24237-00-1 | 2H-Pyran, 6-butyl-3, | Gyrane | 0.18600000 |
| | | 6-dihydro-2,4-dimethyl- | | |
| 100. | 41519-23-7 | Propanoic acid, 2- | Hexenyl | 0.18200000 |
| | | methyl-, (3Z)-3-hexen-1-yl ester | Isobutyrate | |
| 101. | 93-89-0 | Benzoic acid, ethyl ester | Ethyl Benzoate | 0.18000000 |
| 102. | 20780-48-7 | 3-Octanol, 3,7-dimethyl-, | Tetrahydro | 0.18000000 |
| | | 3-acetate | Linalyl Acetate | |
| 103. | 101-41-7 | Methyl 2-phenylacetate | Methylphenyl | 0.17600000 |
| | | | acetate | |
| 104. | 40853-55-2 | 1-Hexanol, 5-methyl- | Tetrahydro | 0.17300000 |
| | | 2-(1-methylethyl)-, 1-acetate | Lavandulyl | |
| | | | Acetate | |
| 105. | 933-48-2 | Cyclohexanol, 3,3,5- | Trimethyl- | 0.17300000 |
| | | trimethyl-, (1R,5R)-rel- | cyclohexanol | |
| 106. | 35158-25-9 | 2-Hexenal, 5-methyl-2- | Lactone of | 0.17200000 |
| | | (1-methylethyl)- | Cis Jasmone | |
| 107. | 18479-58-8 | 7-Octen-2-ol, 2,6-dimethyl- | Dihydromyrcenol | 0.16600000 |
| 108. | 140-11-4 | Acetic acid, phenylmethyl | Benzyl acetate | 0.16400000 |
| | | ester | | |
| 109. | 14765-30-1 | Cyclohexanone, 2- | 2-sec-Butyl Cyclo | 0.16300000 |
| | | (1-methylpropyl)- | Hexanone | |
| 110. | 20125-84-2 | 3-Octen-1-ol, (3Z)- | Octenol | 0.16000000 |
| 111. | 142-19-8 | Heptanoic acid, 2- | Allyl | 0.16000000 |
| | | propen-1-yl ester | Heptoate | |
| 112. | 100-51-6 | Benzenemethanol | Benzyl Alcohol | 0.15800000 |
| 113. | 10032-15-2 | Butanoic acid, 2- | Hexyl-2-Methyl | 0.15800000 |
| | | methyl-, hexyl ester | Butyrate | |
| 114. | 695-06-7 | 2(3H)-Furanone, | Gamma | 0.15200000 |
| | | 5-ethyldihydro- | Hexalactone | |
| 115. | 21722-83-8 | Cyclohexaneethanol, | Cyclohexyl | 0.15200000 |
| | | 1-acetate | Ethyl Acetate | |
| 116. | 111-79-5 | 2-Nonenoic acid, | Methyl-2- | 0.14600000 |
| | | methyl ester | Nonenoate | |
| 117. | 16491-36-4 | Butanoic acid, (3Z)- | Cis 3 Hexenyl | 0.13500000 |
| | | 3-hexen-1-yl ester | Butyrate | |
| 118. | 111-12-6 | 2-Octynoic acid, | Methyl Heptine | 0.12500000 |
| | | methyl ester | Carbonate | |
| 119. | 59323-76-1 | 1,3-Oxathiane, 2-methyl- | Oxane | 0.12300000 |
| | | 4-propyl-, (2R,4S)-rel- | | |
| 120. | 62439-41-2 | Heptanal, 6-methoxy- | Methoxy | 0.11900000 |
| | | 2,6-dimethyl- | Melonal | |
| 121. | 13851-11-1 | Bicyclo[2.2.1]heptan-2-ol, | Fenchyl | 0.11700000 |
| | | 1,3,3-trimethyl-, 2-acetate | Acetate | |
| 122. | 115-95-7 | 1,6-Octadien-3-ol, | Linalyl acetate | 0.11600000 |
| | | 3,7-dimethyl-, 3-acetate | | |
| 123. | 18479-57-7 | 2-Octanol, 2,6-dimethyl- | Tetra-Hydro | 0.11500000 |
| | | | Myrcenol | |
| 124. | 78-69-3 | 3,7-dimethyloctan-3-ol | Tetra-Hydro | 0.11500000 |
| | | | Linalool | |
| 125. | 111-87-5 | 1-Oclanol | Octyl Alcohol | 0.11400000 |
| 126. | 71159-90-5 | 3-Cyclohexene-1- | Grapefruit | 0.10500000 |
| | | methanethiol, α,α,4-trimethyl- | mercaptan | |
| 127. | 80-25-1 | Cyclohexanemethanol, | Menthanyl | 0.10300000 |
| | | α,α,4-trimethyl-, 1-acelate | Acetate | |
| 128. | 88-41-5 | Cyclohexanol, 2-(1,1- | Verdox ™ | 0.10300000 |
| | | dimethylethyl)-, 1-acetate | | |
| 129. | 32210-23-4 | Cyclohexanol, 4-(1,1- | Vertenex | 0.10300000 |
| | | dimethylethyl)-, 1-acetate | | |
| 130. | 112-44-7 | Undecanal | n-Undecanal | 0.10200000 |
| 131. | 24168-70-5 | Pyrazine, 2-methoxy-3- | Methoxy- | 0.09950000 |
| | | (1-methylpropyl)- | isobutylpyrazine | |
| 132. | 89-79-2 | Cyclohexanol, 5-methyl- | Iso-Pulegol | 0.09930000 |
| | | 2-(1-methylethenyl)-, | | |
| | | (1R,2S,5R)- | | |
| 133. | 112-12-9 | 2-Undecanone | Methyl Nonyl | 0.09780000 |
| | | | Ketone | |
| 134. | 103-05-9 | Benzenepropanol, | Phenyl Ethyl | 0.09770000 |
| | | α,α-dimethyl- | Dimethyl | |
| | | | Carbinol | |
| 135. | 125-12-2 | Bicyclo[2.2.1]heptan-2-ol, | Iso Bornyl | 0.09590000 |
| | | 1,7,7-trimethyl-, 2-acetate, | Acetate | |
| | | (1R,2R,4R)-rel- | | |
| 136. | 78-70-6 | l,6-Octadien-3-ol, | Linalool | 0.09050000 |
| | | 3,7-dimethyl- | | |
| 137. | 101-97-3 | Benzeneacetic acid, | Ethyl Phenyl | 0.08970000 |
| | | ethyl ester | Acetate | |
| 138. | 100-86-7 | Benzeneelhanol, | Dimethyl | 0.08880000 |
| | | α,α-dimethyl- | Benzyl Carbinol | |
| 139. | 188570-78-7 | Cyclopropanecarboxylic | Montaverdi | 0.08640000 |
| | | acid, (3Z)-3-hexen-1-yl ester | | |
| 140. | 67634-25-7 | 3-Cyclohexene-1-methanol, | Floralate | 0.08500000 |
| | | 3,5-dimethyl-, 1-acetate | | |
| 141. | 112-44-7 | Undecanal | Undecyl | 0.08320000 |
| | | | Aldehyde | |
| 142. | 32669-00-4 | Ethanone, 1-(3- | Tanaisone ® | 0.08150000 |
| | | cycloocten-1-yl)- | | |
| 143. | 98-53-3 | Cyclohexanone, 4-(1,1- | Patchi | 0.07780000 |
| | | dimethylethyl)- | | |
| 144. | 35854-86-5 | 6-Nonen-1-ol, (6Z)- | cis-6-None-1-ol | 0.07770000 |
| 145. | 5331-14-6 | Benzene, (2-butoxyethyl)- | Butyl phenethyl | 0.07760000 |
| | | | ether | |
| 146. | 80-57-9 | Bicyclo[3.1.1]hept-3- | Verbenone | 0.07730000 |
| | | en-2-one, 4,6,6-trimethyl- | | |
| 147. | 22471-55-2 | Cyclohexanecarboxylic | Thesaron | 0.07670000 |
| | | acid, 2,2,6-trimethyl-, | | |
| | | ethyl ester, (1R,6S)-rel- | | |
| 148. | 60-12-8 | 2-phenyl ethanol | Phenelhyl | 0.07410000 |
| | | | alcohol or | |
| | | | Phenylethyl | |
| | | | alcohol | |
| 149. | 106-26-3 | 2,6-Octadienal, 3,7- | Neral | 0.07120000 |
| | | dimethyl-, (2Z)- | | |
| 150. | 5392-40-5 | 2,6-Octadienal, 3,7- | Citral | 0.07120000 |
| | | dimethyl- | | |
| 151. | 89-48-5 | Cyclohexanol, 5-methyl- | Menthyl | 0.07070000 |
| | | 2-(1-methylethyl)-, 1- | Acetate | |
| | | acetate, (1R,2S,5R)-rel- | | |
| 152. | 119-36-8 | Benzoic acid, 2-hydroxy-, | Methyl | 0.07000000 |
| | | methyl ester | salicylate | |
| 153. | 4180-23-8 | Benzene, 1-methoxy- | Anethol | 0.06870000 |
| | | 4-(1E)-1-propen-1-yl- | | |
| 154. | 7549-37-3 | 2,6-Octadiene, 1,1- | Citral Dimethyl | 0.06780000 |
| | | dimethoxy-3,7-dimethyl- | Acetal | |
| 155. | 25225-08-5 | Cyclohexanemethanol, | Aphermate | 0.06780000 |
| | | α,3,3-trimethyl-, 1-formate | | |
| 156. | 3913-81-3 | 2-Decenal, (2E)- | 2-Decene-1-al | 0.06740000 |
| 157. | 15373-31-6 | 3-Cyclopentene-1-acetonitrile, | Cantryl ® | 0.06700000 |
| | | 2,2,3-trimethyl- | | |
| 158. | 6485-40-1 | 2-Cyclohexen-1-one, 2-methyl- | Laevo | 0.06560000 |
| | | 5-(1-methylethenyl)-, (5R)- | carvone | |
| 159. | 16587-71-6 | Cyclohexanone, 4- | Orivone | 0.06490000 |
| | | (1,1-dimethylpropyl)- | | |
| 160. | 62406-73-9 | 6,10-Dioxaspiro[4.5]decane, | Opalal CI | 0.06290000 |
| | | 8,8-dimethyl-7-(1-methylethyl)- | | |
| 161. | 3720-16-9 | 2-Cyclohexen-1-one, 3- | Livescone | 0.06270000 |
| | | methyl-5-propyl- | | |
| 162. | 13816-33-6 | Benzonitrile, 4-(1- | Cumin | 0.06230000 |
| | | methylethyl)- | Nitrile | |
| 163. | 67019-89-0 | 2,6-Nonadienenitrile | Violet Nitrile | 0.06200000 |
| 164. | 53398-85-9 | Butanoic acid, 2-methyl-, | cis-3-Hexenyl | 0.06130000 |
| | | (3Z)-3-hexen-1-yl ester | Alpha | |
| | | | Methyl Bulyrate | |
| 165. | 208041-98-9 | Heptanenitrile, 2-propyl- | Jasmonitrile | 0.05920000 |
| 166. | 16510-27-3 | Benzene, 1- | Toscanol | 0.05870000 |
| | | (cyclopropylmethyl)-4- | | |
| | | methoxy- | | |
| 167. | 111-80-8 | 2-Nonynoic acid, | Methyl Octine | 0.05680000 |
| | | methyl ester | Carbonate | |
| 168. | 103-45-7 | Acetic acid, 2- | Phenyl Ethyl | 0.05640000 |
| | | phenylethyl ester | Acetate | |
| 169. | 2550-26-7 | 2-Butanone, 4-phenyl- | Benzyl Acetone | 0.05570000 |
| 170. | 13491-79-7 | Cyclohexanol, 2-(1,1- | Verdol | 0.05430000 |
| | | dimethylethyl)- | | |
| 171. | 7786-44-9 | 2,6-Nonadien-1-ol | 2,6-Nonadien-1-ol | 0.05370000 |
| 172. | 103-28-6 | Propanoic acid, 2-methyl-, | Benzyl Iso | 0.05130000 |
| | | phenylmethyl ester | Butyrate | |
| 173. | 104-62-1 | Formic acid, 2- | Phenyl Ethyl | 0.05050000 |
| | | phenylethyl ester | Formate | |
| 174. | 28462-85-3 | Bicyclo[2.2.1]heptan-2-ol, | Humus Ether | 0.04870000 |
| | | 1,2,3,3-tetramethyl-, (1R, | | |
| | | 2R,4S)-rel- | | |
| 175. | 122-03-2 | Benzaldehyde, 4-(1- | Cuminic | 0.04820000 |
| | | methylethyl)- | Aldehyde | |
| 176. | 358331-95-0 | 2,5-Octadien-4-one, 5,6,7- | Pomarose | 0.04810000 |
| | | trimethyl-, (2E)- | | |
| 177. | 562-74-3 | 3-Cyclohexen-1-ol, 4- | Terpinenol-4 | 0.04780000 |
| | | methyl-1-(1-methylethyl)- | | |
| 178. | 68527-77-5 | 3-Cyclohexene-1-methanol, | Isocyclogeraniol | 0.04640000 |
| | | 2,4,6-trimethyl- | | |
| 179. | 35852-46-1 | Pentanoic acid, (3Z)- | Cis-3-Hexenyl | 0.04580000 |
| | | 3-hexen-1-yl ester | Valerate | |
| 180. | 2756-56-1 | Bicyclo[2.2.1]heptan-2-ol, | Iso Bornyl | 0.04540000 |
| | | 1,7,7-trimethyl-, 2- | Propionate | |
| | | propanoate, (1R,2R,4R)-rel- | | |
| 181. | 14374-92-6 | Benzene, 1-methyl-4-(1- | Verdoracine | 0.04460000 |
| | | methylethyl)-2-(1-propen-1-yl)- | | |
| 182. | 6784-13-0 | 3-Cyclohexene-1-propanal, | Limonenal | 0.04380000 |
| | | β,4-dimethyl- | | |
| 183. | 8000-41-7 | 2-(4-methyl-1-cyclohex-3- | Alpha | 0.04320000 |
| | | enyl)propan-2-ol | Terpineol | |
| 184. | 41884-28-0 | 1-Hexanol, 5-methyl- | Tetrahydro | 0.04230000 |
| | | 2-(1-methylethyl)-, (2R)- | Lavandulol | |
| 185. | 22457-23-4 | 3-Heptanone, 5-methyl-, | Stemone ® | 0.04140000 |
| | | oxime | | |
| 186. | 104-50-7 | 2(3H)-Furanone, 5- | Gamma | 0.04080000 |
| | | butyldihydro- | Octalactone | |
| 187. | 143-08-8 | 1-Nonanol | Nonyl Alcohol | 0.04070000 |
| 188. | 3613-30-7 | Octanal, 7-methoxy- | Methoxy- | 0.04020000 |
| | | 3,7-dimethyl- | citronellal | |
| 189. | 67634-00-8 | Acetic acid, 2-(3- | Allyl Amyl | 0.04000000 |
| | | methylbutoxy)-, 2- | Glycolate | |
| | | propen-1-yl ester | | |
| 190. | 464-45-9 | Bicyclo[2.2.1]heptan-2- | 1-Borneol | 0.03980000 |
| | | ol, 1,7,7-trimethyl-,(1S,2R,4S)- | | |
| 191. | 124-76-5 | Bicyclo[2.2.1]heptan-2-ol, | 1.7.7-Trimethyl- | 0.03980000 |
| | | 1,7,7-trimethyl-, (1R,2R,4R)-rel- | Bicyclo-1.2.2- | |
| | | | Heptanol-2 | |
| 192. | 67874-72-0 | Cyclohexanol, 2-(1,1- | Coniferan | 0.03980000 |
| | | dimethylpropyl)-, 1-acetate | | |
| 193. | 80-26-2 | 3-Cyclohexene-1- | Terpinyl | 0.03920000 |
| | | methanol, α,α,4-trimethyl-, | Acetate | |
| | | 1-acetate | | |
| 194. | 498-81-7 | Cyclohexanemethanol, | Dihydro | 0.03920000 |
| | | α,α,4-trimethyl- | Terpineol | |
| 195. | 112-45-8 | 10-Undecenal | Undecylenic | 0.03900000 |
| | | | aldehyde | |
| 196. | 35044-57-6 | 2,4-Cyclohexadiene-1- | Ethyl | 0.03880000 |
| | | carboxylic acid, 2,6,6- | Safranate | |
| | | trimethyl-, ethyl ester | | |
| 197. | 106-21-8 | 1-Octanol, 3,7-dimethyl- | Dimethyl | 0.03860000 |
| | | | Octanol | |
| 198. | 84560-00-9 | Cyclopentanol, 2-pentyl- | Cyclopentol | 0.03790000 |
| 199. | 82461-14-1 | Furan, tetrahydro-2,4- | Rhubafuran ® | 0.03780000 |
| | | dimethyl-4-phenyl- | | |
| 200. | 56011-02-0 | Benzene, [2-(3- | Phenyl Ethyl | 0.03690000 |
| | | methylbutoxy)ethyl]- | Isoamyl Ether | |
| 201. | 103-37-7 | Butanoic acid, | Benzyl | 0.03660000 |
| | | phenylmethyl ester | Butyrate | |
| 202. | 6378-65-0 | Hexyl hexanoate | Hexyl hexanoate | 0.03490000 |
| 203. | 118-61-6 | Benzoic acid, 2-hydroxy-, | Ethyl salicylate | 0.03480000 |
| | | ethyl ester | | |
| 204. | 98-52-2 | Cyclohexanol, 4- | Patchon | 0.03480000 |
| | | (1,1-dimethylethyl)- | | |
| 205. | 115-99-1 | 1,6-Octadien-3-ol, | Linalyl | 0.03440000 |
| | | 3,7-dimethyl-, 3-formate | Formate | |
| 206. | 112-54-9 | Dodecanal | Lauric Aldehyde | 0.03440000 |
| 207. | 53046-97-2 | 3,6-Nonadien-1-ol, (3Z,6Z)- | 3,6 Nonadien-1-ol | 0.03360000 |
| 208. | 76649-25-7 | 3,6-Nonadien-1-ol | 3,6-Nonadien-1-ol | 0.03360000 |
| 209. | 141-25-3 | 3,7-Dimethyloct-6-en-1-ol | Rhodinol | 0.03290000 |
| 210. | 1975-78-6 | Decanenitrile | Decanonitrile | 0.03250000 |
| 211. | 2216-51-5 | Cyclohexanol, 5- | L-Menthol | 0.03230000 |
| | | methyl-2-(1- | | |
| | | methylethyl)-(1R,2S,5R)- | | |
| 212. | 3658-77-3 | 4-hydroxy-2,5- | Pineapple | 0.03200000 |
| | | dimethylfuran-3-one | Ketone | |
| 213. | 103-93-5 | Propanoic acid, 2-methyl-, | Para Cresyl | 0.03120000 |
| | | 4-methylphenyl ester | iso-Butyrate | |
| 214. | 24717-86-0 | Propanoic acid, 2-methyl-, | Abierate | 0.03110000 |
| | | (1R,2S,4R)-1,7,7- | | |
| | | trimethylbicyclo[2.2.1] | | |
| | | hept-2-yl ester, rel- | | |
| 215. | 67845-46-9 | Acetaldehyde, 2-(4- | Aldehyde | 0.03090000 |
| | | methylphenoxy)- | XI | |
| 216. | 67883-79-8 | 2-Butenoic acid, 2-methyl-, | Cis-3-Hexenyl | 0.03060000 |
| | | (3Z)-3-hexen-1-yl ester, (2E)- | Tiglate | |
| 217. | 33885-51-7 | Bicyclo[3.1.1]hept-2- | Pino | 0.03040000 |
| | | ene-2-propanal, 6,6-dimethyl- | Acetaldehyde | |
| 218. | 105-85-1 | 6-Octen-1-ol, 3,7- | Citronellyl | 0.03000000 |
| | | dimethyl-, 1-formate | Formate | |
| 219. | 70214-77-6 | 2-Nonanol, 6,8-dimethyl- | Nonadyl | 0.03010000 |
| 220. | 215231-33-7 | Cyclohexanol, 1-methyl- | Rossitol | 0.02990000 |
| | | 3-(2-methylpropyl)- | | |
| 221. | 120-72-9 | 1H-Indole | Indole | 0.02980000 |
| 222. | 2463-77-6 | 2-Undecenal | 2-Undecene- | 0.02970000 |
| | | | 1-al | |
| 223. | 675-09-2 | 2H-Pyran-2-one, | Levistamel | 0.02940000 |
| | | 4,6-dimethyl- | | |
| 224. | 98-55-5 | 3-Cyclohexene-1- | Alpha- | 0.02830000 |
| | | methanol, α,α,4-trimethyl- | Terpineol | |
| 225. | 81786-73-4 | 3-Hepten-2-one, | Koavone | 0.02750000 |
| | | 3,4,5,6,6-pentamethyl-, (3Z)- | | |
| 226. | 122-97-4 | Benzenepropanol | Phenyl Propyl | 0.02710000 |
| | | | Alcohol | |
| 227. | 39212-23-2 | 2(3H)-Furanone, 5- | Methyl | 0.02700000 |
| | | butyldihydro-4-methyl- | Octalactone | |
| 228. | 53767-93-4 | 7-Octen-2-ol, 2,6-dimethyl-, | Dihydro | 0.02690000 |
| | | 2-acetate | Terpinyl Acetate | |
| 229. | 35044-59-8 | 1,3-Cyclohexadiene-1- | Ethyl | 0.02660000 |
| | | carboxylic acid, 2,6,6- | Safranate | |
| | | trimethyl-, ethyl ester | | |
| 230. | 104-55-2 | 2-Propenal, 3-phenyl- | Cinnamic | 0.02650000 |
| | | | Aldehyde | |
| 231. | 144-39-8 | 1,6-Octadien-3-ol, | I.inalyl | 0.02630000 |
| | | 3,7-dimethyl-, 3-propanoate | Propionate | |
| 232. | 61931-80-4 | 1,6-Nonadien-3-ol, | 3,7-Dimethyl- | 0.02630000 |
| | | 3,7-dimethyl-, 3-acetate | 1,6-nonadien- | |
| | | | 3-yl acetate | |
| 233. | 102-13-6 | Benzeneacetic acid, | Iso Butyl | 0.02630000 |
| | | 2-methylpropyl ester | Phenylacetate | |
| 234. | 65443-14-3 | Cyclopentanone, 2,2,5- | Veloutone | 0.02610000 |
| | | trimethyl-5-pentyl- | | |
| 235. | 141-12-8 | 2,6-Octadien-1-ol, 3,7- | Neryl | 0.02560000 |
| | | dimethyl-, 1-acetate, (2Z)- | Acetate | |
| 236. | 105-87-3 | 2,6-Octadien-1-ol, 3,7- | Geranyl | 0.02560000 |
| | | dimethyl-, 1-acetate, (2E)- | acetate | |
| 237. | 68141-17-3 | Undecane, 1,1- | Methyl Nonyl | 0.02550000 |
| | | dimethoxy-2-methyl- | Acetaldehyde | |
| | | | Dimethyl Acetal | |
| 238. | 2206-94-2 | Benzenemethanol, α- | Indocolore | 0.02550000 |
| | | methylene-, 1-acetate | | |
| 239. | 10528-67-3 | Cyclohexanepropanol, | Cyclohexyl- | 0.02550000 |
| | | α-methyl- | magnol | |
| 240. | 123-11-5 | Benzaldehyde, | Anisic | 0.02490000 |
| | | 4-methoxy- | Aldehyde | |
| 241. | 57576-09-7 | Cyclohexanol, 5- | Iso Pulegol | 0.02480000 |
| | | methyl-2-(1- | Acetate | |
| | | methylethenyl)-, 1- | | |
| | | acetate, (1R,2S,5R)- | | |
| 242. | 51566-62-2 | 6-Octenenitrile, | Citronellyl | 0.02470000 |
| | | 3,7-dimethyl- | Nitrite | |
| 243. | 60335-71-9 | 2H-Pyran, 3,6- | Rosyrane | 0.02470000 |
| | | dihydro-4-methyl-2-phenyl- | Super | |
| 244. | 30385-25-2 | 6-Octen-2-ol, | Dihydro- | 0.02440000 |
| | | 2,6-dimethyl- | myrcenol | |
| 245. | 101-84-8 | Benzene, 1,1′-oxybis- | Diphenyl | 0.02230000 |
| | | | Oxide | |
| 246. | 136-60-7 | Benzoic acid, butyl ester | Butyl Benzoate | 0.02170000 |
| 247. | 93939-86-7 | 5,8-Methano-2H-1- | Rhuboflor | 0.02120000 |
| | | benzopyran, 6- | | |
| | | ethylideneoctahydro- | | |
| 248. | 83926-73-2 | Cyclohexanepropanol | Coranol | 0.02100000 |
| | | dimethyl-, α,α- | | |
| 249. | 125109-85-5 | Benzenepropanal, β- | Florhydral | 0.02070000 |
| | | methyl-3-(1-methylethyl)- | | |
| 250. | 104-21-2 | Benzenemethanol, 4- | Anisyl | 0.02050000 |
| | | methoxy-, 1-acetate | Acetate | |
| 251. | 1365-19-1 | 2-Furanmethanol, 5- | Linalool | 0.02050000 |
| | | ethenyltetrahydro-α,α,5- | Oxide | |
| | | trimethyl- | | |
| 252. | 137-03-1 | Cyclopentanone, 2-heptyl- | Frutalone | 0.02040000 |
| 253. | 2563-07-7 | Phenol, 2-ethoxy-4-methyl- | Ultravanil | 0.02030000 |
| 254. | 1128-08-1 | 2-Cyclopenten-1-one, | Dihydro- | 0.02020000 |
| | | 3-methyl-2-pentyl- | jasmone | |
| 255. | 7493-57-4 | Benzene, [2-(1- | Acetaldehyde | 0.01990000 |
| | | propoxyethoxy (ethyl]- | | |
| 256. | 141-25-3 | 7-Octen-1-ol, 3,7- | Rhodinol | 0.01970000 |
| | | dimethyl- | | |
| 257. | 216970-21-7 | Bicyclo[4.3.1]decane, | 3-Methoxy-7,7- | 0.01960000 |
| | | 3-methoxy-7,7-dimethyl- | dimethyl-10- | |
| | | 10-methylene- | methylenebicyclo | |
| | | | [4.3.1]decane | |
| 258. | 319002-92-1 | Propanoic acid, 2-(1,1- | Sclareolate ® | 0.01960000 |
| | | dimethylpropoxy)-, | | |
| | | propyl ester, (2S)- | | |
| 259. | 85-91-6 | Benzoic acid, 2- | Dimethyl | 0.01930000 |
| | | (methylamino)-, methyl ester | anthranilate | |
| 260. | 13828-37-0 | Cyclohexanemethanol, | Mayol | 0.01920000 |
| | | 4-(1-methylethyl)-, cis- | | |
| 261. | 26330-65-4 | (E)-6-ethyl-3-methyloct- | Super | 0.01850000 |
| | | 6-en-1-ol | Muguet | |
| 262. | 7540-51-4 | 6-Octen-1-ol, | L-Citronellol | 0.01830000 |
| | | 3,7-dimethyl-, (3S)- | | |
| 263. | 106-22-9 | 6-Octen-1-ol, 3,7- | Citronellol | 0.01830000 |
| | | dimethyl- | | |
| 264. | 543-39-5 | 7-Octen-2-ol, 2- | Myrcenol | 0.01820000 |
| | | methyl-6-methylene- | | |
| 265. | 7775-00-0 | Benzenepropanal, | Cyclemax | 0.01820000 |
| | | 4-(1-methylethyl)- | | |
| 266. | 18479-54-4 | 4,6-Octadien-3-ol, | Muguol | 0.01800000 |
| | | 3,7-dimethyl- | | |
| 267. | 29214-60-6 | Octanoic acid, 2- | Gelsone | 0.01790000 |
| | | acetyl-, ethyl ester | | |
| 268. | 1209-61-6 | 5-Oxatricyclo[8.2.0.04,6] | Tobacarol | 0.01730000 |
| | | dodecane, 4,9,12,12- | | |
| | | tetramethyl- | | |
| 269. | 57934-97-1 | 2-Cyclohexene-1- | Givescone | 0.01710000 |
| | | carboxylic acid, 2-ethyl- | | |
| | | 6,6-dimethyl-, ethyl ester | | |
| 270. | 14901-07-6 | 3-Buten-2-one, 4-(2,6,6- | Beta-Ionone | 0.01690000 |
| | | trimethyl-1-cyclohexen-1- | | |
| | | yl)-, (3E)- | | |
| 271. | 64001-15-6 | 4,7-Methano-1H-inden-5- | Dihydro | 0.01630000 |
| | | ol, octahydro-, 5-acetate | Cyclacet | |
| 212. | 95-41-0 | 2-Cyclopenten-1-one, 2-hexyl- | Iso Jasmone T | 0.01600000 |
| 273. | 134-20-3 | Benzoic acid, 2-amino-, | Methyl | 0.01580000 |
| | | methyl ester | Anthranilate | |
| 274. | 100-06-1 | Ethanone, 1-(4- | Para Methoxy | 0.01550000 |
| | | methoxyphenyl)- | Acetophenone | |
| 275. | 105-86-2 | 2,6-Octadien-1-ol, 3,7- | Geranyl | 0.01540000 |
| | | dimethyl-, 1-formate, (2E)- | Formate | |
| 276. | 154171-77-4 | Spiro[1,3-dioxolane-2, | Ysamher K ® | 0.01470000 |
| | | 8′(5′H)-[2H-2,4a] | | |
| | | methanonaphthalene], | | |
| | | hexahydro-1′,1′,5′,5′- | | |
| | | tetramethyl-, (2′S,4′aS,8′aS)- | | |
| | | (9CI) | | |
| 277. | 154171-76-3 | Spiro[1,3-dioxolane-2,8′ | Ysamber | 0.01470000 |
| | | (5′H)-[2H-2,4a] | | |
| | | methanonaphthalene], | | |
| | | hexahydro-1′,1′,5′,5′- | | |
| | | tetramethyl- | | |
| 278. | 127-41-3 | 3-Buten-2-one, 4- | Alpha- | 0.01440000 |
| | | (2,6,6-trimethyl-2- | Ionone | |
| | | cyclohexen-1-yl)-, (3E)- | | |
| 279. | 151-05-3 | Benzeneethanol, α,α- | Dimeihyl | 0.01390000 |
| | | dimethyl-, 1-acetate | Benzyl Carbinyl | |
| | | | Acetate | |
| 280. | 2500-83-6 | 4,7-Methano-1H-inden- | Flor Acetate | 0.01370000 |
| | | 5-ol, 3a,4,5,6,7,7a- | | |
| | | hexahydro-, 5-acetate | | |
| 281. | 150-84-5 | 6-Octen-1-ol, 3,7- | Citronellyl | 0.01370000 |
| | | dimethyl-, 1-acetate | acetate | |
| 282. | 30310-41-9 | 2H-Pyran, tetrahydro- | Pelargene | 0.01350000 |
| | | 2-methyl-4-methylene- | | |
| | | 6-phenyl- | | |
| 283. | 68845-00-1 | Bicyclo[3.3.1]nonane, | Boisiris | 0.01350000 |
| | | 2-ethoxy-2,6,6-trimethyl- | | |
| | | 9-methylene- | | |
| 284. | 106-24-1 | 2,6-Octadien-1-ol, 3,7- | Geraniol | 0.01330000 |
| | | dimethyl-, (2E)- | | |
| 285. | 106-25-2 | 2,6-Octadien-1-ol, 3,7- | Nerol | 0.01330000 |
| | | dimethyl-, (2Z)- | | |
| 286. | 75975-83-6 | Bicyclo[7.2.0]undec-4-ene, | Velyvenal | 0.01280000 |
| | | 4,11,11-trimethyl-8- | | |
| | | methylene-, (1R,4E,9S)- | | |
| 287. | 19870-74-7 | 1H-3a,7-Methanoazulene, | Cedryl | 0.01280000 |
| | | octahydro-6-methoxy- | methyl ether | |
| | | 3,6,8,8-tetramethyl-, | | |
| | | (3R,3aS,6S,7R,8aS)- | | |
| 288. | 87-44-5 | Bicyclo[7.2.0]undec-4-ene, | Caryophyllene | 0.01280000 |
| | | 4,11,11-trimethyl-8- | Extra | |
| | | methylene-, (1R,4E,9S)- | | |
| 289. | 54440-17-4 | 1H-Inden-1-one, 2,3- | Safraleine | 0.01260000 |
| | | dihydro-2,3,3-trimethyl- | | |
| 290. | 110-98-5 | 2-Propanol, 1,1′-oxybis- | Dipropylene | 0.01250000 |
| | | | Glycol | |
| 291. | 41890-92-0 | 2-Octanol, 7-methoxy- | Osyrol ® | 0.01250000 |
| | | 3,7-dimethyl- | | |
| 292. | 71077-31-1 | 4,9-Decadienal, 4,8- | Floral Super | 0.01230000 |
| | | dimethyl- | | |
| 293. | 65-85-0 | Benzoic Acid | Benzoic Acid | 0.01220000 |
| 294. | 61444-38-0 | 3-Hexenoic acid, | cis-3-hexenyl-cis- | 0.01220000 |
| | | (3Z)-3-hexen-1-yl | 3-hexenoate | |
| | | ester, (3Z)- | | |
| 295. | 116044-44-1 | Bicyclo[2.2.1]hept-5-ene- | Herbanate | 0.01210000 |
| | | 2-carboxylic acid, 3-(1- | | |
| | | methylethyl)-, ethyl ester, | | |
| | | (1R,2S,3S,4S)-rel- | | |
| 296. | 104-54-1 | 2-Propen-1-ol, 3-phenyl- | Cinnamic | 0.01170000 |
| | | | Alcohol | |
| 297. | 78-35-3 | Propanoic acid, 2-methyl-, | Linalyl | 0.01170000 |
| | | 1-ethenyl-1,5-dimethyl-4- | Isobutyrate | |
| | | hexen-1-yl ester | | |
| 298. | 23495-12-7 | Ethanol, 2-phenoxy-, 1- | Phenoxy Ethyl | 0.01130000 |
| | | propanoate | Propionate | |
| 299. | 103-26-4 | 2-Propenoic acid, 3- | Methyl Cinnamate | 0.01120000 |
| | | phenyl-, methyl ester | | |
| 300. | 67634-14-4 | Benzenepropanal, 2- | Florazon | 0.01110000 |
| | | ethyl-α,α-dimethyl- | (ortho-isomer) | |
| 301. | 5454-19-3 | Propanoic acid, decyl ester | N-Decyl | 0.01100000 |
| | | | Propionate | |
| 302. | 93-16-3 | Benzene, 1,2-dimethoxy- | Methyl Iso | 0.01100000 |
| | | 4-(1-propen-1-yl)- | Eugenol | |
| 303. | 81782-77-6 | 3-Decen-5-ol, 4-methyl- | 4-Methyl-3- | 0.01070000 |
| | | | decen-5-ol | |
| 304. | 67845-30-1 | Bicyclo[2.2.2]oct-5-ene-2- | Maceal | 0.01060000 |
| | | carboxaldehyde, 6-methyl- | | |
| | | 8-(1-methylethyl)- | | |
| 305. | 97-53-0 | Phenol, 2-methoxy-4- | Eugenol | 0.01040000 |
| | | (2-propen-1-yl)- | | |
| 306. | 120-57-0 | 1,3-Benzodioxole-5- | Heliotropin | 0.01040000 |
| | | carboxaldehyde | | |
| 307. | 93-04-9 | Naphthalene, 2- | Beta Naphthyl | 0.01040000 |
| | | methoxy- | Methyl Ether | |
| | | | Extra 99 | |
| 308. | 4826-62-4 | 2-Dodecenal | 2 Dodecene-1-al | 0.01020000 |
| 309. | 20407-84-5 | 2-Dodecenal, (2E)- | Aldehyde | 0.01020000 |
| | | | Mandarin | |
| 310. | 5462-06-6 | Benzenepropanal, 4- | Canthoxal | 0.01020000 |
| | | methoxy-α-methyl- | | |
| 311. | 94-60-0 | 1,4-Cyclohexane- | Dimethyl 1,4- | 0.01020000 |
| | | dicarboxylic acid, | cyclohexane- | |
| | | 1,4-dimethyl ester | dicarboxylate | |
| 312. | 57378-68-4 | 2-Buten-1-one, 1- | delta-Damascone | 0.01020000 |
| | | (2,6,6-trimethyl-3- | | |
| | | cyclohexen-1-yl)- | | |
| 313. | 17283-81-7 | 2-Butanone, 4-(2,6,6- | Dihydro | 0.01020000 |
| | | trimethyl-1-cyclohexen-1-yl)- | Beta Ionone | |
| 314. | 1885-38-7 | 2-Propenenitrile, 3-phenyl-, | Cinnamalva | 0.01010000 |
| | | (2E) | | |
| 315. | 103-48-0 | Propanoic acid, 2-methyl-, | Phenyl Ethyl | 0.00994000 |
| | | 2-phenylethyl ester | Iso Butyrate | |
| 316. | 488-10-8 | 2-Cyclopenten-1-one, 3- | Cis Jasmone | 0.00982000 |
| | | methyl-2-(2Z)-2-penten-1-yl- | | |
| 317. | 7492-67-3 | Acetaldehyde, 2-[(3,7- | Citronellyloxy- | 0.00967000 |
| | | dimethyl-6-octen-1-yl) oxy]- | acetaldehyde | |
| 318. | 68683-20-5 | 1-Cyclohexene-1-ethanol, 4- | Iso Bergamate | 0.00965000 |
| | | (1-methylethyl)-formate | | |
| 319. | 3025-30-7 | 2,4-Decadienoic acid, ethyl | Ethyl 2,4- | 0.00954000 |
| | | ester, (2E,4Z)- | Decadienoate | |
| 320. | 103-54-8 | 2-Propen-1-ol, 3-phenyl-, | Cinnamyl | 0.00940000 |
| | | 1-acetate | Acetate | |
| 321. | 18127-01-0 | Benzenepropanal, 4- | Bourgeonal | 0.00934000 |
| | | (1,1-dimethylethyl)- | | |
| 322. | 3738-00-9 | Naphtho[2,1-b]furan, | Ambrox ® or | 0.00934000 |
| | | dodecahydro-3a,6,6,9a- | Cetalox ® or | |
| | | tetramethyl- | Synambran | |
| 323. | 51519-65-4 | 1,4-Methanonaphthalen- | Tamisone | 0.00932000 |
| | | 5(1H)-one, 4,4a,6,7,8,8a- | | |
| | | hexahydro- | | |
| 324. | 148-05-1 | Dodecanoic acid, 12- | Dodecalactone | 0.00931000 |
| | | hydroxy-, λ-lactone (6CI, | | |
| | | 7CI); 1,12- | | |
| 325. | 6790-58-5 | (3aR,5aS,9aS,9bR)- | Ambronat ® or | 0.00930000 |
| | | 3a,6,6,9a-tetramethyl- | Ambroxan ® | |
| | | 2,4,5,5a,7,8,9,9b-octahydro- | | |
| | | 1H-benzo[e][1]benzofuran | | |
| 326. | 86-26-0 | 1,1′-Biphenyl, 2-methoxy- | Methyl | 0.00928000 |
| | | | Diphenyl Ether | |
| 327. | 68738-94-3 | 2-Naphthalenecarboxaldehyde, | Cyclomyral ® | 0.00920000 |
| | | octahydro-8,8-dimethyl | | |
| 328. | 2705-87-5 | Cyclohexanepropanoic | Allyl | 0.00925000 |
| | | acid, 2-propen-1-yl ester | Cyclohexane | |
| | | | Propionate | |
| 329. | 7011-83-8 | 2(3H)-Furanone, 5- | Lactojasmone ® | 0.00885000 |
| | | hexyldihydro-5-methyl- | | |
| 330. | 61792-11-8 | 2,6-Nonadienenitrile, | Lemonile ® | 0.00884000 |
| | | 3,7-dimethyl- | | |
| 331. | 692-86-4 | 10-Undecenoic acid, | Ethyl | 0.00882000 |
| | | ethyl ester | Undecylenate | |
| 332. | 103-95-7 | Benzenepropanal, α- | Cymal | 0.00881000 |
| | | methyl-4-(1-methylethyl)- | | |
| 333. | 13019-22-2 | 9-Decen-1-ol | Rosalva | 0.00879000 |
| 334. | 94201-19-1 | 1-Oxaspiro[4.5]decan-2- | Methyl Laitone | 0.00872000 |
| | | one, 8-methyl- | 10% TEC | |
| 335. | 104-61-0 | 2(3H)-Furanone, | γ-Nonalactone | 0.00858000 |
| | | dihydro-5-pentyl- | | |
| 336. | 706-14-9 | 2(3W)-Furanone, 5- | γ-Decalactone | 0.00852000 |
| | | hexyldihydro- | | |
| 337. | 24720-09-0 | 2-Buten-1-one, 1-(2,6,6- | α-Damascone | 0.00830000 |
| | | trimethyl-2-cyclohexen- | | |
| | | 1 yl)-, (2E)- | | |
| 338. | 39872-57-6 | 2-Buten-1-one, 1- | Isodamascone | 0.00830000 |
| | | (2,4,4-trimethyl-2- | | |
| | | cyclohexen-1-yl)-,(2E)- | | |
| 339. | 705-86-2 | 2H-Pyran-2-one, | Decalactone | 0.00825000 |
| | | tetrahydro- 6-pentyl- | | |
| 340. | 67634-15-5 | Benzenepropanal, | Floralozone | 0.00808000 |
| | | 4-ethyl-α,α-dimethyl- | | |
| 341. | 40527-42-2 | 1,3-Benzodioxole, 5- | Heliotropin | 0.00796000 |
| | | (diethoxymethyl)- | Diethyl Acetal | |
| 342. | 56973-85-4 | 4-Penten-1-one, 1- | Neobutenone α | 0.00763000 |
| | | (5,5-dimethyl-1- | | |
| | | cyclohexen-1-yl)- | | |
| 343. | 128-51-8 | Bicyclo[3.1.1]hept-2- | Nopyl | 0.00751000 |
| | | ene-2-ethanol-6,6- | Acetate | |
| | | dimethyl-, 2-acetate | | |
| 344. | 103-36-6 | 2-Propenoic acid, 3- | Ethyl | 0.00729000 |
| | | phenyl-, ethyl ester | Cinnamate | |
| 345. | 5182-36-5 | 1,3-Dioxane, 2,4,6- | Floropal ® | 0.00709000 |
| | | trimethyl-4-phenyl- | | |
| 346. | 42604-12-6 | Cyclododecane, | Boisambrene | 0.00686000 |
| | | (methoxymethoxy)- | | |
| 347. | 33885-52-8 | Bicycio[3.1.l]hept-2- | Pinyl Iso | 0.00685000 |
| | | ene-2-propanal, α,α,6,6- | Butyrate | |
| | | tetramethyl- | Alpha | |
| 348. | 92015-65-1 | 2(3H)-Benzofuranone, | Natactone | 0.00680000 |
| | | hexahydro-3,6-dimethyl- | | |
| 349. | 63767-86-2 | Cyclohexanemethanol, α- | Mugetanol | 0.00678000 |
| | | methyl-4-(1-methylethyl)- | | |
| 350. | 3288-99-1 | Benzeneacetonitrile, 4- | Marenil CI | 0.00665000 |
| | | (1,1-dimethylethyl)- | | |
| 351. | 35044-68-9 | 2-Buten-1-one, 1-(2,6,6- | beta- | 0.00655000 |
| | | trimethyl-1-cyclohexen-1-yl)- | Damascone | |
| 352. | 41724-19-0 | 1,4-Methanonaphthalen- | Plicatone | 0.00652000 |
| | | 6(2H)-one, octahydro-7- | | |
| | | methyl- | | |
| 353. | 75147-23-8 | Bicyclo[3.2.1]oclan-8-one, | Buccoxime ® | 0.00647000 |
| | | 1,5-dimethyl-, oxime | | |
| 354. | 25634-93-9 | 2-Methyl-5-phenylpentan- | Rosaphen ® | 0.00637000 |
| | | 1-ol | 600064 | |
| 355. | 55066-48-3 | 3-Methyl-5-phenylpentanol | Phenyl | 0.00637000 |
| | | | Hexanol | |
| 356. | 495-62-5 | Cyclohexene, 4-(1,5- | Bisabolene | 0.00630000 |
| | | dimethyl-4-hexen-1- | | |
| | | ylidene)-1-methyl- | | |
| 357. | 2785-87-7 | Phenol, 2-methoxy-4-propyl- | Dihydro | 0.00624000 |
| | | | Eugenol | |
| 358. | 87-19-4 | Benzoic acid, 2-hydroxy-, | Iso Butyl | 0.00613000 |
| | | 2-methylpropyl ester | Salicylate | |
| 359. | 4430-31-3 | 2H-1-Benzopyran-2-one, | Octahydro | 0.00586000 |
| | | octahydro- | Coumarin | |
| 360. | 38462-22-5 | Cyclohexanone, 2-(1- | Ringonol | 0.00585000 |
| | | mercapto-1-methylethyl)- | 50 TEC | |
| | | 5-methyl- | | |
| 361. | 77-83-8 | 2-Oxiranecarboxylic | Ethyl Methyl | 0.00571000 |
| | | acid, 3-methyl-3-phenyl-, | Phenyl | |
| | | ethyl ester | Glycidate | |
| 362. | 37677-14-8 | 3-Cyclohexene-1- | Iso Hexenyl | 0.00565000 |
| | | carboxaldehyde, 4-(4- | Cyclohexenyl | |
| | | methyl-3-penten-1-yl)- | Carboxaldehyde | |
| 363. | 103-60-6 | Propanoic acid, | Phenoxy Ethyl | 0.00562000 |
| | | 2-methyl-, 2-phenoxyethyl | iso-Butyrate | |
| | | ester | | |
| 364. | 18096-62-3 | Indenol[1,2-d]-1,3-dioxin, | Indoflor ® | 0.00557000 |
| | | 4,4a,5,9b-tetrahydro- | | |
| 365. | 63500-71-0 | 2H-Pyran-4-ol, tetrahydro- | Florosa Q/ | 0.00557000 |
| | | 4-methyl-2-(2-methylpropyl)- | Florol | |
| 366. | 65405-84-7 | Cyclohexanebutanal, | Cetonal ® | 0.00533000 |
| | | α,2,6,6-tetramethyl- | | |
| 367. | 171102-41-3 | 4,7-Methano-1H-inden-6-ol, | Flor Acetate | 0.00530000 |
| | | 3a,4,5,6,7,7a-hexahydro-8,8- | | |
| | | dimethyl-, 6-acetate | | |
| 368. | 10339-55-6 | 1,6-Nonadien-3-ol, 3,7- | Ethyl linalool | 0.00520000 |
| | | dimethyl- | | |
| 369. | 23267-57-4 | 3-Buten-2-one, 4-(2,2,6- | Ionone | 0.00520000 |
| | | trimethyl-7-oxabicyclo[4.1.0] | Epoxide Beta | |
| | | hept-1-yl)- | | |
| 370. | 97-54-1 | Phenol, 2-methoxy-4-(1- | Isoeugenol | 0.00519000 |
| | | propen-1-yl)- | | |
| 371. | 67663-01-8 | 2(3H)-Furanone, 5- | Peacholide | 0.00512000 |
| | | hexyldihydro-4-methyl- | | |
| 372. | 33885-52-8 | Bicyclo[3.1.1]hept-2-ene-2- | Pinyl Iso | 0.00512000 |
| | | propanal, α,α,6,6-tetramethyl- | Butyrate | |
| | | | Alpha | |
| 373. | 23696-85-7 | 2-Buten-1-one, 1-(2,6,6- | Damascenone | 0.00503000 |
| | | trimethyl-1,3-cyclohexadien- | | |
| | | 1-yl)- | | |
| 374. | 80-71-7 | 2-Cyclopenten-1-one, 2- | Maple | 0.00484000 |
| | | hydroxy-3-methyl- | Lactone | |
| 375. | 67662-96-8 | Propanoic acid, 2,2-dimethyl-, | Pivarose Q | 0.00484000 |
| | | 2-phenylethyl ester | | |
| 376. | 2437-25-4 | Dodecanenitrile | Clonal | 0.00480000 |
| 377. | 141-14-0 | 6-Octen-1-ol, 3,7-dimethyl-, | Citronellyl | 0.00469000 |
| | | 1-propanoate | Propionate | |
| 378. | 54992-90-4 | 3-Buten-2-one, 4- | Myrrhone | 0.00460000 |
| | | (2,2,3,6-tetramethylcyclohexyl)- | | |
| 379. | 55066-49-4 | Benzenepentanal, β-methyl- | Mefranal | 0.00455000 |
| 380. | 7493-74-5 | Acetic acid, 2-phenoxy-, | Allyl Phenoxy | 0.00454000 |
| | | 2-propen-1-yl ester | Acetate | |
| 381. | 80-54-6 | Benzenepropanal, 4-(1,1- | Lilial ® | 0.00444000 |
| | | dimethylethyl)-α-methyl- | | |
| 382. | 86803-90-9 | 4,7-Methano-1H-indene-2- | Scenlenal ® | 0.00439000 |
| | | carboxaldehyde, octahydro- | | |
| | | 5-methoxy- | | |
| 383. | 68991-97-9 | 2-Naphthalenecarboxaldehyde, | Melafleur | 0.00436000 |
| | | 1,2,3,4,5,6,7,8-octahydro-8,8- | | |
| | | dimethyl- | | |
| 384. | 18871-14-2 | Pentitol, 1,5-anhydro-2,4- | Jasmal | 0.00434000 |
| | | dideoxy-2-pentyl-, 3-acetate | | |
| 385. | 58567-11-6 | Cyclododecane, | Boisambren | 0.00433000 |
| | | (ethoxymethoxy)- | Forte | |
| 386. | 94400-98-3 | Naphth[2,3-b]oxirene, | Molaxone | 0.00425000 |
| | | 1a,2,3,4,5,6,7,7a-octahydro- | | |
| | | 1a,3,3,4,6,6-hexamethyl-, | | |
| | | (1aR,4S,7aS)-rel- | | |
| 387. | 79-69-6 | 3-Buten-2-one, 4-(2,5,6,6- | alpha-Irone | 0.00419000 |
| | | tetramethyl-2-cyclohexen-1-yl)- | | |
| 388. | 65442-31-1 | Quinoline, 6-(1-methylpropyl)- | Iso Butyl | 0.00408000 |
| | | | Quinoline | |
| 389. | 87731-18-8 | Carbonic acid, 4-cycloocten- | Violiff | 0.00401000 |
| | | 1-yl methyl ester | | |
| 390. | 173445-65-3 | 1H-Indene-5-propanal, | Hivernal | 0.00392000 |
| | | 2,3-dihydro-3,3-dimethyl- | (A-isomer) | |
| 391. | 23911-56-0 | Ethanone, 1-(3-methyl-2- | Nerolione | 0.00383000 |
| | | benzofuranyl)- | | |
| 392. | 52474-60-9 | 3-Cyclohexene-1- | Precyclemone | 0.00381000 |
| | | carboxaldehyde, 1-methyl- | B | |
| | | 3-(4-methyl-3-penten-1-yl) | | |
| 393. | 139539-66-5 | 6-Oxabicyclo[3.2.1]octane, | Cassifix | 0.00381000 |
| | | 5-methyl-1-(2,2,3-trimethyl- | | |
| | | 3-cyclopenlen-1-yl)- | | |
| 394. | 80858-47-5 | Benzene, [2- | Phenafleur | 0.00380000 |
| | | (cyclohexyloxy)ethyl]- | | |
| 395. | 32764-98-0 | 2H-Pyran-2-one, | Jasmolactone | 0.00355000 |
| | | tetrahydro-6-(3-penten-1-yl)- | | |
| 396. | 78417-28-4 | 2,4,7-Decatrienoic | Ethyl 2,4,7- | 0.00353000 |
| | | acid, ethyl ester | decatrienoate | |
| 397. | 140-26-1 | Butanoic acid, 3- | Beta Phenyl | 0.00347000 |
| | | methyl-, 2-phenylethyl ester | Ethyl | |
| | | | Isovalerate | |
| 398. | 105-90-8 | 2,6-Octadien-1-ol, 3,7- | Geranyl | 0.003360000 |
| | | dimethyl-, 1-propanoate, (2E)- | Propionate | |
| 399. | 41816-03-9 | Spiro[1,4-methanonaphthalene- | Rhubofix ® | 0.00332000 |
| | | 2(1H),2′-oxirane], 3,4,4a,5,8,8a- | | |
| | | hexahydro-3′,7-dimethyl- | | |
| 400. | 7070-15-7 | Ethanol, 2-[[(1R,2R,4R)- | Arbanol | 0.00326000 |
| | | 1,7,7-trimethylbicyclo[2.2.1] | | |
| | | hept-2-yl]oxy]-, rel- | | |
| 401. | 93-29-8 | Phenol, 2-methoxy-4-(1- | Iso Eugenol | 0.00324000 |
| | | propen-1-yl)-, 1-acetate | Acetate | |
| 402. | 476332-65-7 | 2H-Indeno[4,5-b]furan, | Amber Xtreme | 0.00323000 |
| | | decahydro-2,2,6,6,7,8,8- | Compound 1 | |
| | | heptamethyl- | | |
| 403. | 68901-15-5 | Acetic acid, 2-(cyclohexyloxy)-, | Cyclogalbanate | 0.00323000 |
| | | 2-propen-1-yl ester | | |
| 404. | 107-75-5 | Octanal, 7-hydroxy-3,7- | Hydroxy- | 0.00318000 |
| | | dimethyl- | citronellal | |
| 405. | 68611-23-4 | Naphtho[2,1-b]furan, 9b- | Grisalva | 0.00305000 |
| | | ethyldodecahydro- | | |
| | | 3a,7,7-trimethyl- | | |
| 406. | 313973-37-4 | 1,6-Heptadien-3-one, | Pharaone | 0.00298000 |
| | | 2-cyclohexyl- | | |
| 407. | 137-00-8 | 5-Thiazoleethanol, 4-methyl- | Sulfurol | 0.00297000 |
| 408. | 7779-30-8 | 1-Penten-3-one, 1-(2,6,6- | Methyl Ionone | 0.00286000 |
| | | trimethyl-2-cyclohexen-1-yl)- | | |
| 409. | 127-51-5 | 3-Buten-2-one, 3-methyl-4- | Isoraldeine | 0.00282000 |
| | | (2,6,6-trimethyl-2- | Pure | |
| | | cyclohexen-1-yl)- | | |
| 410. | 72903-27-6 | 1,4-Cyclohexane- | Fructalate ™ | 0.00274000 |
| | | dicarboxylic acid, | | |
| | | 1,4-diethyl ester | | |
| 411. | 7388-22-9 | 3-Buten-2-one, 4-(2,2- | Ionone | 0.00272000 |
| | | dimethyl-6- | Gamma | |
| | | methylenecyclohexyl)- | | |
| | | 3-methyl- | Methyl | |
| 412. | 104-67-6 | 2(3H)-Furanone, | gamma- | 0.00271000 |
| | | 5-heptyldihydro- | Undecalactone | |
| | | | (racemic) | |
| 413. | 1205-17-0 | 1,3-Benzodioxole-5-propanal, | Helional | 0.00270000 |
| | | α-methyl- | | |
| 414. | 33704-61-9 | 4H-Inden-4-one, 1,2,3,5,6,7- | Cashmeran | 0.00269000 |
| | | hexahydro-1,1,2,3,3-pentamethyl- | | |
| 415. | 36306-87-3 | Cyclohexanone, 4-(1- | Kephalis | 0.00269000 |
| | | ethoxyethenyl)-3,3,5,5-tetramethyl- | | |
| 416. | 97384-48-0 | Benzenepropanenitrile, α- | Citrowanil ® | 0.00265000 |
| | | ethenyl-α-methyl- | B | |
| 417. | 141-13-9 | 9-Undecenal, 2,6,10-trimelhyl- | Adoxal | 0.00257000 |
| 418. | 2110-18-1 | Pyridine, 2-(3-phenylpropyl)- | Corps | 0.00257000 |
| | | | Racine VS | |
| 419. | 27606-09-3 | Indeno[1,2-d]-1,3-dioxin, | Magnolan | 0.00251000 |
| | | 4,4a,5,9b-tetrahydro-2,4-dimethyl- | | |
| 420. | 67634-20-2 | Propanoic acid. 2-methyl-, | Cyclabute | 0.00244000 |
| | | 3a,4,5,6,7,7a-hexahydro-4,7- | | |
| | | methano-1H-inden-5-yl ester | | |
| 421. | 65405-72-3 | 1-Naphthalenol, 1,2,3,4,4a,7,8,8a- | Oxyoctaline | 0.00236000 |
| | | octahydro-2,4a,5,8a-tetramethyl-, | Formate | |
| | | 1-formate | | |
| 422. | 122-40-7 | Heptanal, 2-(phenylmethylene)- | Amyl Cinnamic | 0.00233000 |
| | | | Aldehyde | |
| 423. | 103694-68-4 | Benzenepropanol, β,β,3-trimethyl- | Majantol ® | 0.00224000 |
| 424. | 13215-88-8 | 2-Cyclohexen-1-one, 4-(2-buten- | Tabanone | 0.00223000 |
| | | 1-ylidene)-3,5,5-trimethyl- | Coeur | |
| 425. | 25152-85-6 | 3-Hexen-1-ol, 1-benzoate, (3Z)- | Cis-3-Hexenyl | 0.00203000 |
| | | | Benzoate | |
| 426. | 406488-30-0 | 2-Ethyl-N-methyl-N-(m- | Paradisamide | 0.00200000 |
| | | tolyl)butanamide | | |
| 427. | 121-33-5 | Benzaldehyde, 4-hydroxy-3- | Vanillin | 0.00194000 |
| | | methoxy- | | |
| 428. | 77-54-3 | 1H-3a,7-Methanoazulen-6-ol, | Cedac | 0.00192000 |
| | | octahydro-3,6,8,8-tetramethyl-, | | |
| | | 6-acetate, (3R,3aS,6R,7R,8aS)- | | |
| 429. | 76842-49-4 | 4,7-Methano-1H-inden-6-ol, | Frutene | 0.00184000 |
| | | 3a,4,5,6,7,7a-hexahydro-8,8- | | |
| | | dimethyl-, 6-propanoate | | |
| 430. | 121-39-1 | 2-Oxiranecarboxylic | Ethyl Phenyl | 0.00184000 |
| | | acid, 3-phenyl-, ethyl ester | Glycidale | |
| 431. | 211299-54-6 | 4H-4a,9-Methanoazuleno[5,6-d]- | Ambrocenide ® | 0.00182000 |
| | | 1,3-dioxole, octahydro- | | |
| | | 2,2,5,8,8,9a-hexamethyl-, | | |
| | | (4aR,5R,7aS,9R)- | | |
| 432. | 285977-85-7 | (2,5-Dimethyl-1,3-dihydroinden- | Lilyflore | 0.00180000 |
| | | 2-yl)methanol | | |
| 433. | 10094-34-5 | Butanoic acid, 1,1-dimethyl-2- | Dimethyl | 0.00168000 |
| | | phenylethyl ester | Benzyl | |
| | | | Carbinyl | |
| | | | Bulyrate | |
| 434. | 40785-62-4 | Cyclododeca[c]furan, | Muscogene | 0.00163000 |
| | | 1,3,3a,4,5,6,7,8,9,10,11,13a- | | |
| | | dodecahydro- | | |
| 435. | 75490-39-0 | Benzenebutanenitrile, α,α,γ- | Khusinil | 0.00162000 |
| | | trimethyl- | | |
| 436. | 55418-52-5 | 2-Butanone, 4-(1,3-benzodioxol- | Dulcinyl | 0.00161000 |
| | | 5-yl)- | | |
| 437. | 3943-74-6 | Benzoic acid, 4-hydroxy-3- | Carnaline | 0.00157000 |
| | | methoxy-, methyl ester | | |
| 438. | 72089-08-8 | 3-Cyclopentene-1-butanol, | Brahmanol ® | 0.00154000 |
| | | β,2,2,3-tetramethyl-2- | | |
| | | Methyl-4-(2,2,3-trimethyl-3- | | |
| | | cyclopenten-1-yl)butanol | | |
| 439. | 3155-71-3 | 2-Butenal, 2-methyl-4-(2,6,6- | Boronal | 0.00147000 |
| | | trimethyl-1-cyclohexen-1-yl)- | | |
| 440. | 2050-08-0 | Benzoic acid, 2-hydroxy-, | Amyl | 0.00144000 |
| | | pentyl ester | Salicylate | |
| 441. | 41199-20-6 | 2-Naphthalenol, decahydro- | Ambrinol | 0.00140000 |
| | | 2,5,5-trimethyl- | | |
| 442. | 12262-03-2 | ndecanoic acid, 3- | Iso Amyl | 0.00140000 |
| | | methylbutyl ester | Undecylenate | |
| 443. | 107-74-4 | 1,7-Octanediol, 3,7-dimethyl- | Hydroxyol | 0.00139000 |
| 444. | 91-64-5 | 2H-1-Benzopyran-2-one | Coumarin | 0.00130000 |
| 445. | 68901-32-6 | 1,3-Dioxolane. 2-[6-methyl-8- | Glycolierral | 0.00121000 |
| | | (1-methylethyl) bicyclo[2.2.2] | | |
| | | oct-5-en-2-yl]- | | |
| 446. | 68039-44-1 | Propanoic acid, 2,2-dimethyl-, | Pivacyclene | 0.00119000 |
| | | 3a,4,5,6,7,7a-hexahydro-4,7- | | |
| | | methano-1H-inden-6-yl ester | | |
| 447. | 106-29-6 | Butanoic acid, (2E)-3,7- | Geranyl | 0.00116000 |
| | | dimethyl-2,6-octadien-1-yl ester | Butyrate | |
| 448. | 5471-51-2 | 2-Butanone, 4-(4-hydroxyphenyl)- | Raspberry | 0.00106000 |
| | | | ketone | |
| 449. | 109-42-2 | 10-Undecenoic acid, butyl ester | Butyl | 0.00104000 |
| | | | Undecylenate | |

*Vapor Pressures are acquired as described in the Test Methods Section.
**Origin: Same as tor Table 2 hereinabove.

### Test Methods

The following assays set forth must be used in order that the invention described and claimed herein may be more fully understood.

### Test Method 1: Determining Vapor Pressure

In order to determine the vapor pressure for the fragrance materials, go to the website https://scifinder.cas.org/scifinder/view/scifinder/scifinderExplore.jsf and follow these steps to acquire the vapor pressure.

1. Input the CAS registry number for the particular fragrance material.

2. Select the vapor pressure from the search results.

3. Record the vapor pressure (given in Torr at 25° C.).

SciFinder uses Advanced Chemistry Development (ACD/Labs) Software Version 14.02). If the CAS number for the particular fragrance material is unknown or does not exist, you can utilize the ACD/Labs reference program to directly determine the vapor pressure. Vapor Pressure is expressed in 1 Torr, which is equal to 0.133 kilopascal (kPa).

### Test Method 2: Olfactory Tests

In order to show the effect of the substantially non-odorous fragrance fixatives on the perception of fragrance profile in a composition of the present invention, test compositions are made, as described in the Example section, and given to panelists to evaluate.

At the testing facility, 50 μL samples of the compositions and the controls are applied to glass slides and placed on a hot plate at 32° C. to represent skin temperature for varying durations. It is important that glass slides of samples that are to be later compared are prepared at the same time. The panelists are asked to evaluate the perceived fragrance profile (intensity and/or character) of each glass slide sample at a given time-point. Slides are presented coded so that their identity is not known by the panelists. Within a given time point panelists evaluate the slides in a random order and are able to revisit their assessment as they work through the slides at that time point. Their assessments are recorded. In the subsequent analysis, the data for strength and character comparisons are drawn from the independent assessments carried out at a given time point. Only when using the difference scale below are any two products physically directly compared to each other. Panelists are selected from individuals who are either trained to evaluate fragrances according to the scales below or who have experience with fragrance evaluation in the industry. Typically, around 6 to 10 panelists are used to evaluate a given product and its control.

(a) Fragrance Intensity:

The panelists are asked to give a score on a scale of 0 to 5 for perceived fragrance intensity according to the odour intensity scale set out in Table 4 herein below.

TABLE 4
Odour Intensity Scale

| Score | Fragrance Intensity |
|---|---|
| 0 | None |
| 1 | Very Weak |
| 2 | Weak |
| 3 | Moderate |
| 4 | Strong |
| 5 | Very Strong |

(b) Fragrance Character:

The panelists are asked to assess the fragrance character in one of 2 ways:
i) a score on a scale of 0 to 3 for the dominance of particular characters that are relevant to that particular fragrance, e.g.: fresh, green, watery, floral, rose, muguet, fruity, apple, berry, citrus, creamy, woody, balsamic, amber, musk just to name a few, according to the odour grading scale set out in Table 5(i) herein below;
ii) a score on a scale of 1 to 5 for changes in the perceived fragrance profile change for the test compositions versus the controls according to the odour grading scale set out in Table 5(ii) herein below.

TABLE 5(i)
Character Dominance Odour Grading Scale

| Score | Fragrance Character Dominance |
|---|---|
| 0 | Not noticeable |
| 1 | Slight presence of the character |
| 2 | Moderate presence of the character |
| 3 | Dominance of the character |

TABLE 5(ii)
Character Difference Odour Grading Scale

| Score | Fragrance Character Change |
|---|---|
| 1 | Fragrance character is unchanged, i.e., no difference between the |
| | sample vs. the control. |
| 2 | Slight fragrance character change when compared directly with |
| | the control. |
| 3 | Moderate fragrance change but similar character to the control. |
| 4 | Large difference in fragrance character from the control. |
| 5 | Total difference in the fragrance character from the control. |

The results of the panelists are averaged and then analysed using Analysis of Variance methods. The model treats the subject as a random effect and looks at the impact of product, time and the interaction between product and time. From the analysis the least square means for the product and time interaction are obtained. These means (as well as their confidence intervals) are then plotted to enable comparisons between products at each time point. It should be noted that the confidence levels plotted are intended as a guide, and not as a statistical comparison, as they do not take into account that multiple testing has been performed. As well as a graphical assessment, statistical comparisons between the two products at each of the time points are performed with a Tukey correction for multiple comparisons. The p-values for the product differences were obtained, with p-values<0.05 indicating a statistical difference between the two products at 5% significance (or 95% confidence).

### Test Method 3: Analytical Evaporation Tests

The following test is carried out to demonstrate the improved or enhanced longevity of a fragrance profile of a composition of the present invention vs. a control. In particular, the test measures the effect of a substantially non-odorous fragrance fixative on the evaporation rate of one or more fragrance materials (e.g., 10 PRMs) formulated in a composition. The evaporation response of the fragrance materials to the fixative, as a function of time, is measured through the use of gas chromatography (“GC”).
1. A test composition may comprise a substantially non-odorous fragrance fixative (as disclosed in Table 1) with either: (i) a fragrance material (any one of the fragrance materials disclosed in Table 3) or (ii) a blend of fragrance materials from Table 3 (as disclosed as Fragrance Example 6 in Table 11). The test composition may also comprise high purity ethanol, such as Hayman 100% EP/BP grade, and (optionally) deionised water. Sample test compositions are provided in Tables 18(d), 19(b) and 19(c). All of the ingredients are admixed until evenly distributed in the test compositions.
2. A control composition to the test composition described in 1 above, without the substantially non-odorous fragrance fixative, is made in a similar manner to Step 1, except that the missing substantially non-odorous fragrance fixative is replaced by deionized water. Sample control compositions are provided in Tables 18(d), 19(b) and 19(c).
3. An internal standard is needed to correct for variations of the amount of composition dispensed in the evaporation test as well as loss during the GC analysis. The internal standard has a vapor pressure of less than 0.001 Torr (0.000133 kPa) at 25° C. and is soluble in the composition or fragrance material. A suitable non-limiting example of internal standard is triethyl citrate. The internal standard and fragrance material are admixed until evenly distributed at a level of 90 to 95 parts by weight of fragrance material and the required amount of internal standard to reach 100 parts. This mixture is then use to prepare the sample compositions in Step 1 and 2. Alternatively, the internal standard and test or control composition are admixed until evenly distributed at a level of 99 to 99.75 parts by weight of composition and the required amount of internal standard to reach 100 parts. This resultant solution is used in subsequent steps.
4. A hotplate is set to a temperature of 32° C. An aluminium container, such as TA instrument Tzero™ pan is placed on the hotplate. 20 μL of the test or control composition is introduced in the aluminium container using a micropipette. Alternatively, the aluminium container may be filled with the test or control composition to its full capacity. The time at which this takes place is determined to be time zero (i.e., T=0). Multiple aluminium containers are prepared and left at the set temperature for pre-determined periods of time, such as for example 30 mins, 1 hr, 2 hrs, 3 hrs, 4 hrs, 5 hrs, 6 hrs, 8 hrs and up to 12 hrs.
5. The aluminium container is removed from the hotplate at the end of the pre-determined time period and its content transferred into a 4 mL glass vial already containing 2 mL of highly volatile solvent, such as high purity ethanol or hexane.
6. The glass vial is mixed using a Heidolph multi REAX shaker, or equivalent, for 5 to 10 mins to extract the fragrance materials into the solvent phase. 1.5 mL of the resultant solution is transferred to a 2 mL GC vial.
7. The GC vial is analysed on an Agilent GC system 6890 equipped with an autosampler, or equivalent. A GC column such as a DB-5MS, Rxi-5 SilMS model, with a length of 30 m, an inner diameter of 0.25 mm and a film thickness of 1 μm is used. The GC parameters are set to the values indicated as follows:

TABLE 5(iii)
GC Parameters

| | |
|---|---|
| Injector temperature: | 270° C. |
| Initial gas velocity: | 30 to 40 cm/sec (for Helium as the carrier |
| | gas) |
| Injection type: | Split |
| Initial oven temperature: | 50° C. for 1 min |
| Temperature ramp: | 8° C./min |
| Final oven temperature: | 310° C. |

Gas chromatography with flame ionisation detection (“FID”) or with mass spectrometry (“MS”) can be used for the identification and quantification of aroma chemicals in the compositions. Either detection system can be used in conjunction with GC. The column dimensions as well as GC settings described in this method, such as injector temperature, carrier gas velocity, temperature ramp and final oven temperature can be adjusted to optimize the response of the fragrance material and internal standard being monitored. The detection system settings, such as FID gas flows and temperature or MS parameters, should be optimized by a trained analyst to enable the precise detection and quantification of the analytes of interest.
8. The peak area of the fragrance material and internal standard are recorded. The peak area ratio of the fragrance material and the internal standard is calculated at each time point for each sample composition. The % loss of non-evaporated fragrance material remaining from T=0 is calculated at each time point for each sample composition. The % of non-evaporated fragrance material from T=0 calculated. The % fragrance material remaining in each composition is plotted to give an evaporation profile over time. This is done for both the test and control compositions. Significance is determined by comparison of the evaporation profile for the same fragrance material or same fragrance mixture in the test and control compositions.

### Test Method 4: Analytical Headspace Tests

The following test is carried out to demonstrate the character retention over time of a fragrance composition of the present invention vs. a control. It is necessary for the test and control samples to be run at approximately the same time to ensure that ambient conditions are the same. The test measures the presence of one or more fragrance materials in the headspace formed in a sealed vial by the test composition, after set evaporation times. The fragrance profile in the headspace is measured at specific time points through the use of headspace (“HS”) gas chromatography (“GC”).
1. The test and control compositions as described in the Example section are used for the evaluation.
2. Capillaries of about 2 cm to 3.5 cm, with one sealed end are cut from a Sigma Aldrich “Stuart™ melting point tube” product code Z673269, or equivalent. A suitable fixed volume chosen between 50 and 200 μL of the composition is pipetted into the well of a WVR Tissue Culture 96 F well plate, or equivalent. The sealed end of the glass capillary is dipped into the filled well and left for at least 15 secs to wet the surface of the glass. Care must be taken not to contact the glass capillary with the sides of the well by maintaining it straight and approximately in the center of the well.
3. The glass capillary is then removed from the well and inverted or transferred onto a stable surface or into a holder and allowed to evaporate at ambient conditions for a set period of time. A windshield may be used to reduce high air turbulence.
4. The glass capillary is then introduced into an empty 20 mL HS vial, which is immediately closed with a PTFE cap. The time at which this takes place is determined to be time T=initial (i.e., T=10 mins)
5. Multiple glass capillaries are prepared in the same way and left to evaporate at ambient temperature for pre-determined periods of time, such as for example 10, 15, 30 mins, 1 hr, 2 hrs, 3 hrs, 4 hrs, 5 hrs, and up to 6 hrs, before being introduced to the headspace vial and sealed.
6. The HS vial is then analysed on an Agilent GC system 6890 equipped with a Gerstel MPS 2 autosampler, or equivalent, capable of performing SPME injections. A SPME fiber assembly DVB/CAR/PDMS (50/30 μm, 1 cm length) is required. A GC column such as a DB-5MS, ZB-SMSi models, or equivalent phase, with a length of 30 m, an inner diameter of 0.25 mm and a film thickness of 1 μm is used.
7. The SPME HS parameters are set to the values indicated as follows:

TABLE 5(iv)
SPME Parameters

| | |
|---|---|
| Incubation chamber temperature: | 40° C. |
| Incubation time: | 20 mins |
| Agitation of sample | 250 RPM |
| Extraction time | 5 mins |
| Desorption time | 2 mins |

8. The GC parameters are set to the values indicated as follows:

TABLE 5(v)
GC Parameters

| | |
|---|---|
| Injector temperature: | 270° C. |
| Initial gas velocity: | 20 to 40 cm/sec (for Helium as the carrier |
| | gas) |
| Initial oven temperature: | 45° C. with 2 mins Hold Time |
| Temperature ramp 1: | 30° C./min |
| Temperature 1: | 80° C. |
| Temperature ramp 2: | 8° C./min |
| Final temperature: | 300° C. |

Gas chromatography with flame ionization detection (“FID”) or with mass spectrometry (“MS”) can be used for the identification and quantification of fragrance material in the compositions. Either detection system can be used in conjunction with GC. The column dimensions as well as GC settings described in this method, such as injector temperature, carrier gas velocity, temperature ramp and final oven temperature can be adjusted to optimize the response of the fragrance material being monitored. The detection system settings, such as FID gas flows and temperature or MS parameters, should be optimized by a trained analyst to enable the precise detection and identification of the analytes of interest.
9. A qualitative assessment of the chromatograms obtained is performed by comparing the peak height of the fragrance materials and overall chromatogram at time T=10 mins to other time points. A dotted line is drawn around an estimated retention time where fragrance materials with a vapour pressure of 0.001 Torr or less (0.000133 kPa or less) elute during the analysis. The difference between the peaks present at each measured time point for the test and control compositions provides evidence of the retention of the character of the fragrance over time.
10. This test set-up is designed to enable the collection of the headspace in a manner that does not saturate the SPME fiber. If the fiber is saturated it does not provide an accurate analysis of the headspace composition. Therefore the quantity of liquid and the evaporation surface area are very different from those in the olfactive evaluation of the same samples. For this reason it is not possible to compare directly the evaporation time frames used in the 2 experiments. It is expected that the evaporation profile is much faster in this headspace experiments compared to the olfactive evaluations.

### EXAMPLES

The following examples are provided to further illustrate the present invention and are not to be construed as limitations of the present invention, as many variations of the present invention are possible without departing from its spirit or scope.

### Example 1

### Fragrance Oils

Fragrance examples 1, 2, 3, 4b and 5b are provided below in Tables 6, 7, 8, 9 and 10, respectively, as non-limiting examples of formulations of fragrance materials intended to form the fragrance component of the compositions of the present invention. The exemplary formulations of the fragrance materials span the range from “simple accords” (less than 10 fragrance materials) to “complex fragrances” (greater than 30 fragrance materials). Typically, full bodied fragrance compositions do not comprise less than about 30 fragrance materials.

Fragrance examples 4a and 5a provided in Table 9 and 10, respectively, below are examples of traditional formulations of fragrance materials that fall outside the scope of the present invention.

Fragrance example 6 provided in Table 11 below as an example of a formulation of volatile fragrance materials.

Fragrance examples 7 and 8 are provided in Tables 12 and 13 below as examples of a formulation of fragrance materials intended to form the fragrance component that fall outside the scope of the present invention.

Fragrance examples 9 to 16 are provided in Tables 14 and 15 below as examples of formulations of fragrance materials containing higher than 30 wt % of the low volatile fragrance materials.

Fragrance examples 17 and 18 are provided in Tables 16 and 17 below as comparative samples of formulations of fragrance materials intended to form the fragrance component.

TABLE 6
Fragrance Example 1 (Fresh Floral Accord - 10 wt % of Low Volatile
Fragrance Materials)

| | | Vapor Pressure | Parts |
|---|---|---|---|
| Ingredients | CAS Number | (Torr at 25° C.) | (wt %) |
| Benzyl acetate | 140-11-4 | 0.1640 | 10.8 |
| Linalool | 78-70-6 | 0.0905 | 9.8 |
| Phenethyl alcohol | 60-12-8 | 0.0741 | 15.7 |
| Indole | 120-72-9 | 0.0298 | 1.0 |
| α-Terpineol | 98-55-5 | 0.0283 | 2.9 |
| Geranyl acetate | 105-87-3 | 0.0256 | 4.9 |
| Cymal | 103-95-7 | 0.00881 | 5.9 |
| Hydroxycitronellal | 107-75-5 | 0.00318 | 22.4 |
| Majantol | 103694-68-4 | 0.00224 | 16.6 |
| Hexyl cinnamic | 101-86-0 | 0.000697 | 10.0 |
| aldehyde | | | |

| | |
|---|---|
| Total | 100.00 |

TABLE 7
Fragrance Example 2 (Fresh Male Accord - 13.51 wt % of Low Volatile
Fragrance Materials)

| | | Vapor Pressure | Parts |
|---|---|---|---|
| Ingredients | CAS Number | (Torr at 25° C.) | (wt %) |
| d-Limonene | 5989-27-5 | 1.540000 | 10.0 |
| Dihydromyrcenol | 18479-58-8 | 0.166000 | 10.0 |
| Boisiris | 68845-00-1 | 0.013500 | 6.5 |
| Canthoxal | 5462-06-6 | 0.010200 | 8.0 |
| Helional | 1205-17-0 | 0.002700 | 10.0 |
| Kephalis | 36306-87-3 | 0.002690 | 20.0 |
| Majantol | 103694-68-4 | 0.002240 | 15.5 |
| Javanol ® | 198404-98-7 | 0.000902 | 5.0 |
| Galaxolide ®* | 1222-05-5 | 0.000414 | 7.5 |
| Isopropyl | 110-27-0 | — | 7.5 |
| Myristate | | | |

| | |
|---|---|
| Total | 100.00 |

*Supplied at 50% in Isopropyl myristate.

TABLE 8
Fragrance Example 3 (Sweet Dream 18 Fragrance - 11.15 wt % of
Low Volatile Fragrance Materials)

| | | Vapor Pressure | Parts |
|---|---|---|---|
| Ingredients | CAS Number | (Torr at 25° C.) | (wt %) |
| Prenyl acetate | 1191-16-8 | 3.99000000 | 0.100 |
| Manzanate | 39255-32-8 | 2.91000000 | 0.200 |
| Hexyl acetate | 142-92-7 | 1.39000000 | 0.700 |
| cis-3-Hexenyl | 3681-71-8 | 1.22000000 | 0.200 |
| acetate | | | |
| Benzaldehyde | 100-52-7 | 0.97400000 | 0.200 |
| Liffarome | 67633-96-9 | 0.72100000 | 0.150 |
| Hexyl isobutyrate | 2349-07-7 | 0.41300000 | 0.055 |
| Dihydromyrcenol | 18479-58-8 | 0.16600000 | 2.500 |
| Benzyl acetate | 140-11-4 | 0.16400000 | 0.700 |
| Linalyl acetate | 115-95-7 | 0.11600000 | 2.500 |
| Verdox | 88-41-5 | 0.10300000 | 4.000 |
| Phenethyl alcohol | 60-12-8 | 0.07410000 | 8.000 |
| Rossitol | 215231-33-7 | 0.02990000 | 1.500 |
| alpha-Terpineol | 98-55-5 | 0.02830000 | 1.500 |
| Geranyl acetate | 105-87-3 | 0.02560000 | 1.500 |
| Rhodinol | 141-25-3 | 0.01970000 | 0.700 |
| Givescone | 57934-97-1 | 0.01710000 | 0.700 |
| Methyl anthranilate | 134-20-3 | 0.01580000 | 0.050 |
| Ysamber K | 154171-77-4 | 0.01470000 | 1.000 |
| alpha-Ionone | 127-41-3 | 0.01440000 | 3.000 |
| Citronellyl acetate | 150-84-5 | 0.01370000 | 0.500 |
| cis-3-hexenyl-cis-3- | 61444-38-0 | 0.01220000 | 0.200 |
| hexenoate | | | |
| Cinnamic alcohol | 104-54-1 | 0.01170000 | 0.100 |
| delta-damascone | 57378-68-4 | 0.01020000 | 0.200 |
| Citronellyloxyacetal | 7492-67-3 | 0.00967000 | 0.100 |
| dehyde | | | |
| Cymal | 103-95-7 | 0.00881000 | 0.500 |
| Floralozone | 67634-15-5 | 0.00808000 | 0.100 |
| Ethylmethylphenylg | 77-83-8 | 0.00571000 | 0.200 |
| lycidate | | | |
| Florosa Q | 63500-71-0 | 0.00557000 | 3.000 |
| Ethyl linalool | 10339-55-6 | 0.00520000 | 6.400 |
| Pivarose | 67662-96-8 | 0.00484000 | 2.500 |
| Hydroxycitronellal | 107-75-5 | 0.00318000 | 7.500 |
| Methyl Ionone | 7779-30-8 | 0.00286000 | 4.000 |
| gamma- | 104-67-6 | 0.00271000 | 0.500 |
| Undecalactone | | | |
| Kephalis | 36306-87-3 | 0.00269000 | 5.000 |
| Cashmeran | 33704-61-9 | 0.00269000 | 1.000 |
| Magnolan | 27606-09-3 | 0.00251000 | 3.000 |
| Majantol | 103694-68-4 | 0.00224000 | 6.900 |
| Brahmanol | 72089-08-8 | 0.00154000 | 3.000 |
| Coumarin | 91-64-5 | 0.00130000 | 0.500 |
| Glycolierral | 68901-32-6 | 0.00121000 | 0.100 |
| Raspberry ketone | 5471-51-2 | 0.00106000 | 0.100 |
| Top Mango base ³ | — | — | 0.500 |
| Cherry base ³ | — | — | 0.200 |
| Cassis base ³ | — | — | 0.300 |
| Bergamot Oil ⁴ | — | — | 6.000 |
| Prunella base ³ | — | — | 0.500 |
| Hexyl cinnamic | 101-86-0 | 0.00069700 | 1.500 |
| aldehyde | | | |
| Sandalore | 65113-99-7 | 0.00062500 | 3.000 |
| Dupical | 30168-23-1 | 0.00044100 | 0.005 |
| Galaxolide ®¹ | 1222-05-5 | 0.00041400 | 1.500 |
| Ebanol | 67801-20-1 | 0.00028100 | 2.000 |
| Helvetolide | 141773-73-1 | 0.00005790 | 2.000 |
| Warm Milk base ⁵ | — | — | 0.200 |
| Vanilla Absolute ^{2, 6} | — | — | 0.100 |
| Isopropyl Myristate | — | — | 1.500 |
| Dipropylene Glycol | — | — | 6.040 |
| | | Total | 100.00 |

¹Supplied at 50% in IPM.
² Supplied at 50% in DiPG.
³ Proprietary bases that contain a mixture of perfume raw materials, judged to be of high volatility for the purposes of calculating % of low volatility PRMs.
⁴ Natural oils or extracts that contain a mixture of perfume raw materials, judged to be of high volatility for the purposes of calculating % of low volatility PRMs.
⁵ Proprietary bases that contain a mixture of perfume raw materials, judged to be of low volatility for the purposes of calculating % of low volatility PRMs.
⁶ Natural oils or extracts that contain a mixture of perfume raw materials, judged to be of low volatility for the purposes of calculating % of low volatility PRMs.

TABLE 9
Fragrance Examples 4a and 4b (″Traditional Floral Magnifica″ Example 4a -
37 wt % of Low Volatile Fragrance Materials and ″Reduced Low Volatile Floral Magnifica″
Example 4b - 13 wt % of Low Volatile Fragrance Materials)

| | | | Parts (wt %) | |
|---|---|---|---|---|
| | | | | Example 4b |
| | CAS | Vapor Pressure | Example 4a | (Reduced |
| Ingredients | Number | (Torr at 25° C.) | (Traditional) | Low Volatile) |
| Beta Gamma Hexenol | 928-96-1 | 2.126000 | 0.20 | 0.20 |
| Cis 3 Hexenyl Acetate | 3681-71-8 | 1.219000 | 0.30 | 0.30 |
| Benzyl Acetate | 140-11-4 | 0.16400000 | 3.01 | 3.01 |
| Liffarome | 67633-96-9 | 0.721000 | 0.20 | 0.20 |
| Ligustral Or Triplal | 68039-49-6 | 0.578000 | 0.10 | 0.10 |
| Methyl Pamplemousse | 67674-46-8 | 0.214000 | 0.40 | 0.40 |
| d-Limonene | 5989-27-5 | 1.54000000 | 3.01 | 3.01 |
| Phenyl Acetaldehyde ¹ | 122-78-1 | 0.368000 | 0.0002 | 0.0002 |
| Precyclemone B | 52475-86-2 | 0.003810 | 0.20 | 0.20 |
| Ethyl 2 4- | 3025-30-7 | 0.009540 | 0.20 | 0.20 |
| Decadienoate | | | | |
| Ambronat | 6790-58-5 | 0.009340 | 2.00 | 2.01 |
| Alpha Damascone | 24720-09-0 | 0.008300 | 0.04 | 0.06 |
| Citronellol | 106-22-9 | 0.032900 | 4.01 | 4.01 |
| Cyclemax | 7775-00-0 | 0.018200 | 0.40 | 0.40 |
| Cyclo Galbanate | 68901-15-5 | 0.003230 | 0.10 | 0.10 |
| Cymal | 103-95-7 | 0.008810 | 0.90 | 1.51 |
| Dimethyl Benzyl | 10094-34-5 | 0.001680 | 0.50 | 0.50 |
| Carbinyl Butyrate | | | | |
| Ethyl Linalool | 10339-55-6 | 0.005200 | 7.23 | 12.04 |
| Florol | 63500-71-0 | 0.005570 | 6.43 | 10.71 |
| Gamma Decalactone | 706-14-9 | 0.008520 | 0.20 | 0.20 |
| Geraniol | 106-24-1 | 0.013300 | 3.01 | 5.02 |
| Geranyl Acetate | 105-87-3 | 0.009760 | 2.01 | 2.01 |
| Helional | 1205-17-0 | 0.002700 | 2.41 | 4.01 |
| Heliotropin | 120-57-0 | 0.010400 | 0.20 | 0.20 |
| Hivernal | 173445-65-3 | 0.00392000 | 0.20 | 0.20 |
| Hydroxycitronellal | 107-75-5 | 0.003180 | 2.41 | 4.01 |
| Ionone Beta | 14901-07-6 | 0.003080 | 0.24 | 0.40 |
| Ionone Gamma | 127-51-5 | 0.002820 | 1.81 | 3.01 |
| Methyl | | | | |
| Jasmal | 18871-14-2 | 0.004340 | 5.02 | 5.02 |
| Jasmolactone | 32764-98-0 | 0.003550 | 0.20 | 0.20 |
| Linalyl Propionate | 144-39-8 | 0.026300 | 1.20 | 1.20 |
| Magnolan 690304 | 27606-09-3 | 0.002510 | 3.01 | 5.02 |
| Majantol | 103694-68-4 | 0.002240 | 2.41 | 4.01 |
| Phenyl Ethyl Alcohol | 60-12-8 | 0.074100 | 3.01 | 5.02 |
| Phenyl Hexanol | 55066-48-3 | 0.006370 | 3.61 | 6.02 |
| Undecavertol | 81782-77-6 | 0.010700 | 2.01 | 2.01 |
| Vanillin | 121-33-5 | 0.001940 | 0.10 | 0.10 |
| cis-3-Hexenyl cis-3- | 61444-38-0 | 0.012200 | 0.10 | 0.10 |
| Hexenoate | | | | |
| Phenoxy Ethyl Iso | 103-60-6 | 0.005620 | 0.50 | 0.50 |
| Butyrate | | | | |
| 5-Cyclohexadecen-1- | 37609-25-9 | 0.000033 | 1.00 | 1.00 |
| One | | | | |
| Ambrettolide | 28645-51-4 | 0.000001 | 1.00 | 1.00 |
| Cis-3-Hexenyl | 65405-77-8 | 0.000246 | 1.51 | 0.50 |
| Salicylate | | | | |
| Delta Muscenone | 63314-79-4 | 0.000165 | 1.00 | 1.00 |
| 962191 | | | | |
| Hedione ® HC | 24851-98-7 | 0.000710 | 10.54 | 3.51 |
| Iso-E Super ® | 54464-57-2 | 0.000538 | 10.54 | 3.51 |
| Para Hydroxy Phenyl | 5471-51-2 | 0.001060 | 0.20 | 0.20 |
| Butanone | | | | |
| Polysantol | 107898-54-4 | 0.000117 | 0.50 | 0.50 |

| | | |
|---|---|---|
| Total | 100 | 100 |

TABLE 10
Fragrance Examples 5a and 5b (″Traditional Muguesia Magnifica″ Example
5a - 37 wt % of Low Volatile Fragrance Materials and ″Reduced Low Volatile Muguesia
Magnifica″ Example 5b - 13 wt % of Low Volatile Fragrance Materials)

| | | | Parts (wt %) | |
|---|---|---|---|---|
| | | | | Example 5b |
| | CAS | Vapor Pressure | Example 5a | (Reduced |
| Ingredients | Number | (Torr at 25° C.) | (Traditional) | Low Volatile) |
| Benzyl Alcohol | 100-51-6 | 0.158000 | 0.10 | 0.10 |
| Methyl Phenyl Carbinyl | 93-92-5 | 0.203000 | 0.32 | 0.40 |
| Acetate | | | | |
| d-Limonene | 5989-27-5 | 1.54000000 | 1.00 | 1.00 |
| Benzyl Acetate | 140-11-4 | 0.304000 | 5.86 | 7.32 |
| Beta Gamma Hexenol | 928-96-1 | 2.126000 | 0.40 | 0.40 |
| Cis 3 Hexenyl Acetate | 3681-71-8 | 1.219000 | 0.20 | 0.20 |
| Linalyl Acetate | 115-95-7 | 0.077400 | 1.00 | 1.00 |
| Jasmal | 18871-14-2 | 0.004340 | 3.21 | 4.01 |
| Indol | 120-72-9 | 0.029800 | 0.10 | 0.10 |
| Hydroxycitronellal | 107-75-5 | 0.003180 | 3.21 | 4.01 |
| Helional | 1205-17-0 | 0.002700 | 4.01 | 5.02 |
| Geranyl Acetate | 105-87-3 | 0.009760 | 3.21 | 4.01 |
| Geraniol | 106-24-1 | 0.013300 | 4.01 | 5.02 |
| Florosa Q | 63500-71-0 | 0.005570 | 0 | 9.03 |
| Cinnamic Alcohol | 104-54-1 | 0.005720 | 0.20 | 0.20 |
| Cinnamic Aldehyde | 104-55-2 | 0.02650000 | 0.06 | 0.06 |
| Cis Jasmone | 488-10-8 | 0.020100 | 0.50 | 0.50 |
| Citronellol | 106-22-9 | 0.032900 | 4.01 | 5.01 |
| Citronellyl Acetate | 150-84-5 | 0.013700 | 3.21 | 4.01 |
| Citronellyl | 7492-67-3 | 0.009670 | 0.10 | 0.10 |
| Oxyacetaldehyde | | | | |
| Cyclemax | 7775-00-0 | 0.018200 | 0.32 | 0.40 |
| Cyclo Galbanate | 68901-15-5 | 0.003230 | 0.20 | 0.20 |
| Cymal | 103-95-7 | 0.008810 | 1.61 | 2.01 |
| Ethyl Linalool | 10339-55-6 | 0.005200 | 8.04 | 10.03 |
| Florhydral | 125109-85-5 | 0.020700 | 0.16 | 0.20 |
| Majantol | 103694-68-4 | 0.002240 | 3.21 | 4.01 |
| Phenyl Ethyl Acetate | 103-45-7 | 0.056400 | 0.40 | 0.40 |
| Phenyl Ethyl Alcohol | 60-12-8 | 0.074100 | 14.45 | 18.06 |
| Ambrettolide | 28645-51-4 | 0.000001 | 1.00 | 1.00 |
| Cis-3-Hexenyl | 65405-77-8 | 0.000246 | 1.00 | 0.50 |
| Salicylate | | | | |
| Benzyl Salicylate | 118-58-1 | 0.00017500 | 16.61 | 2.51 |
| Hedione ® HC | 24851-98-7 | 0.000710 | 8.03 | 4.01 |
| Iso-E Super ® | 54464-57-2 | 0.000538 | 10.03 | 5.02 |
| Phenyl Acetaldehyde | 101-48-4 | 0.55600000 | 0.20 | 0.10 |
| Dimethyl Acetal | | | | |

| | | |
|---|---|---|
| Total | 100 | 100 |

TABLE 11
Fragrance Example 6 (10 Volatile Fragrance Materials)

| | | Vapor Pressure | Parts |
|---|---|---|---|
| Ingredients | CAS Number | (Torr at 25° C.) | (wt %) |
| Tetra-Hydro | 78-69-3 | 0.115 | 9.85 |
| Linalool | | | |
| Terpinyl acetate | 80-26-2 | 0.0392 | 12.21 |
| Dimethyl Benzyl | 151-05-3 | 0.0139 | 11.96 |
| Carbinyl Acetate | | | |
| Dimethyl Benzyl | 100-86-7 | 0.088844 | 9.35 |
| Carbinol | | | |
| Phenyl Ethyl | 60-12-8 | 0.074100 | 7.60 |
| alcohol | | | |
| Laevo Carvone | 6485-40-1 | 0.0656 | 9.35 |
| Indole | 120-72-9 | 0.0298 | 7.29 |
| Ethyl Safranate | 35044-59-8 | 0.0266 | 12.09 |
| Indocolore | 2206-94-2 | 0.0255 | 10.09 |
| Eugenol | 97-53-0 | 0.0104 | 10.21 |

| | |
|---|---|
| Total | 100.00 |

TABLE 12
Fragrance Example 7 (Fresh Floral GF 6-7 Accord - 40.14 wt % of Low
Volatile Fragrance Materials)

| | | Vapor Pressure | Parts |
|---|---|---|---|
| Ingredients | CAS Number | (Torr at 25° C.) | (wt %) |
| Ligustral or | 68039-49-6 | 0.578000 | 0.15 |
| Triplal | | | |
| Benzyl acetate | 140-11-4 | 0.164000 | 0.31 |
| Verdox | 88-41-5 | 0.103000 | 5.38 |
| Phenethyl alcohol | 60-12-8 | 0.074100 | 1.54 |
| Indole | 120-72-9 | 0.029800 | 0.02 |
| Heliotropin | 120-57-0 | 0.010400 | 1.23 |
| gamma- | 706-14-9 | 0.008520 | 0.38 |
| Decalactone | | | |
| Florosa Q | 63500-71-0 | 0.005570 | 15.38 |
| Ethyl linalool | 10339-55-6 | 0.005200 | 26.15 |
| Isoeugenol | 97-54-1 | 0.005190 | 0.08 |
| alpha-Irone | 79-69-6 | 0.004190 | 1.54 |
| Vanillin | 121-33-5 | 0.001940 | 6.15 |
| Dimethyl benzyl | 10094-34-5 | 0.001680 | 1.54 |
| carbinyl butyrate | | | |
| Methyl beta- | 93-08-3 | 0.000957 | 0.77 |
| naphthyl ketone | | | |
| Methyl | 24851-98-7 | 0.000710 | 30.60 |
| dihydrojasmonate | | | |
| Benzyl salicylate | 118-58-1 | 0.000175 | 7.69 |
| Polysantol | 107898-54-4 | 0.000117 | 0.77 |
| Lrg 201 | 4707-47-5 | 0.000029 | 0.31 |
| | | Total | 100.00 |

TABLE 13
Fragrance Example 8 (Traditional Floral Accord - 54.00 wt % of Low
Volatile Fragrance Materials)

| | | Vapor Pressure | Parts |
|---|---|---|---|
| Ingredients | CAS Number | (Torr at 25° C.) | (wt %) |
| Benzyl acetate | 140-11-4 | 0.1640 | 5.5 |
| Linalool | 78-70-6 | 0.0905 | 5.0 |
| Phenethyl alcohol | 60-12-8 | 0.0741 | 8.0 |
| Indole | 120-72-9 | 0.0298 | 0.5 |
| α-Terpineol | 98-55-5 | 0.0283 | 1.5 |
| Geranyl acetate | 105-87-3 | 0.0256 | 2.5 |
| Cymal | 103-95-7 | 0.00881 | 3.0 |
| Hydroxycitronellal | 107-75-5 | 0.00318 | 11.5 |
| Majantol | 103694-68-4 | 0.00224 | 8.5 |
| Hexyl cinnamic | 101-86-0 | 0.000697 | 4.0 |
| aldehyde | | | |
| iso gamma super | 68155-66-8 | 0.000565 | 12.50 |
| Sandalore | 65113-99-7 | 0.000625 | 18.75 |
| Habanolide | 111879-80-2 | 0.00000431 | 18.75 |

| | |
|---|---|
| Total | 100.00 |

TABLE 14
Fragrance Examples 9, 10, 11 and 12 (Traditional Flora Magnifica -
Greater than 30 wt % of Low Volatile Fragrance Materials)

| | Fragrance | Fragrance | Fragrance | Fragrance |
|---|---|---|---|---|
| | Example | Example | Example | Example |
| | 9 | 10 | 11 | 12 |
| Ingredients | Weight % | Weight % | Weight % | Weight % |
| Flora Magnifica ¹ | 86.96 | 83.33 | 74.07 | 68.97 |
| Ethylene | 4.35 | 4.167 | 3.704 | 6.90 |
| Brassylate | | | | |
| Methyl Dihydro | 4.35 | 8.33 | 14.82 | 13.79 |
| Jasmonate | | | | |
| Iso-E Super ® | 4.35 | 4.167 | 7.407 | 10.35 |
| Total | 100 | 100 | 100 | 100 |
| Wt % Low Volatile | 45 | 47 | 53 | 56 |
| Fragrance Materials | | | | |

¹ Fragrance Example 4a.

TABLE 15
Fragrance Examples 13, 14, 15 and 16 (Traditional Muguesia
Magnifica - Greater than 30 wt % of Low Volatile Fragrance
Materials)

| | Fragrance | Fragrance | Fragrance | Fragrance |
|---|---|---|---|---|
| | Example | Example | Example | Example |
| | 13 | 14 | 15 | 16 |
| Ingredients | Weight % | Weight % | Weight % | Weight % |
| Muguesia Magnifica ¹ | 86.96 | 83.33 | 74.07 | 68.97 |
| Ethylene | 4.35 | 4.17 | 3.70 | 6.90 |
| Brassylate | | | | |
| Methyl Dihydro | 4.35 | 8.33 | 14.82 | 13.79 |
| Jasmonate | | | | |
| Iso-E Super ® | 4.35 | 4.17 | 7.41 | 10.35 |
| Total | 100 | 100 | 100 | 100 |
| Wt % Low Volatile | 45 | 47 | 53 | 56 |
| Fragrance Materials | | | | |

¹ Fragrance Example 5a.

Fragrance example 17 (as disclosed in Table 16) is composed of 68.51 wt % of volatile fragrance materials and 31.49 wt % of low volatile fragrance materials, wherein the wt % is relative to the total weight of the fragrance component.

TABLE 16
Fragrance Example 17 (Comparative Fragrance 1 - 31.49 wt % of Low
Volatile Fragance Materials)

| | | | Amount | |
|---|---|---|---|---|
| | CAS | Vapor Pressure | Parts by | Parts |
| Ingredients | Number | (Torr at 25° C.) | Weight | (wt %) |
| Limonene | 5989-27-5 | 1.541 | 2576 | 30.04 |
| Cis-3-Hexenol | 928-96-1 | 1.039 | 21 | 0.24 |
| Zestover ⁶ | 78-70-6 | 0.578 | 1 | 0.01 |
| Linalol | 78-70-6 | 0.0905 | 553 | 6.45 |
| Aphermate ⁴ (10% DIPG) ⁷ | 25225-08-5 | 0.0678 | 7 | 0.08 |
| Cyclosal | 535-86-4 | 0.0311 | 35 | 0.41 |
| Coranol | 83926-73-2 | 0.0210 | 371 | 4.33 |
| Sclareolate ®* ¹ | 319002-92-1 | 0.0196 | 630 | 7.35 |
| 3-Methoxy-7,7-dimethyl-l 0- | 216970-21-7 | 0.0196 | 371 | 4.33 |
| methylene-bicyclo[4.3.1] | | | | |
| decane | | | | |
| Cedramber ² | 19870-74-8 | 0.0128 | 1050 | 12.24 |
| Ambrox ®* | 6790-58-5 | 0.00934 | 1 | 0.01 |
| Decal | 706-14-9 | 0.00852 | 21 | 0.24 |
| Damascone Alpha* (10% DIPG) ⁷ | 24720-09-0 | 0.00830 | 9.1 | 0.11 |
| (Methoxymethoxy)Cyclododecane | 42604-12-6 | 0.00686 | 182 | 2.12 |
| Lilial ® | 80-54-6 | 0.00444 | 26 | 0.30 |
| γ-Undecalactone* | 104-67-6 | 0.00271 | 21 | 0.24 |
| Calone ®* 3 | 28940-11-6 | 0.000831 | 50 | 0.58 |
| Paradisone ⁵®* | 24851-98-7 | 0.000710 | 1000 | 11.66 |
| Galaxolide ® (70% MIP Extra) ⁷ | 1222-05-5 | 0.000414 | 700 | 8.16 |
| Exaltenone | 14595-54-1 | 0.0000964 | 950 | 11.08 |

| | | |
|---|---|---|
| Total | 8575.10 | 100 wt % |

*origin: Firmenich SA (Geneva, Switzerland).
¹ Propyl (S)-2-(1,1-dimethylpropxy)propanoate.
² 8-Methoxy-2,6,6,8-tetramethyl-tricyclo[5.3.1.0(1,5)]undecane.
³ 7-Methyl-2H,4H-1,5-benzodioxepin-3-one.
⁴ 1-(3,3-dimethyl-1-cyclohexyl)ethyl formate; origin: International Flavors & Fragrances.
⁵ Methyl dihydrojasmonate.
⁶ Linalool.
⁷ Fragrance materials added as dilutions in a non-volatile solvent. For the purposes of calculating the fragrance oil composition actual fragrance materials levels added are used.

Fragrance example 18 (as disclosed in Table 17) is composed of 90.63 wt % of volatile fragrance materials and 9.37 wt % of low volatile fragrance materials, wherein the wt % is relative to the total weight of the fragrance component.

TABLE 17
Fragrance Example 18 (Comparative Fragrance 2 - 9.37 wt % of Low Volatile
Fragance Materials)

| | | | Amount | |
|---|---|---|---|---|
| | CAS | Vapor Pressure | Parts by | Parts |
| Ingredients | Number | (Torr at 25° C.) | Weight | (wt %) |
| D-Limonene | 5989-27-5 | 1.540 | 50.00 | 5.21 |
| cis-3-Hexenol (10% in DPG) ⁴ | 928-96-1 | 1.040 | 0.5 | 0.05 |
| Acetophenone (10% in DPG) ⁴ | 98-86-2 | 0.299 | 1.00 | 0.10 |
| Methylphenyl Acetate | 101-41-7 | 0.176 | 10.00 | 1.04 |
| Dihydromyrcenol | 18479-58-8 | 0.166 | 50.00 | 5.21 |
| Benzyl acetate | 140-11-4 | 0.164 | 60.00 | 6.25 |
| Tetra-Hydro Linalool | n/a | 0.115 | 50.00 | 5.21 |
| n-Undecanal | n/a | 0.102 | 5.00 | 0.52 |
| Linalool | 78-70-6 | 0.0905 | 40.00 | 4.17 |
| Phenylethyl Alcohol | n/a | 0.0559 | 245.00 | 25.53 |
| Allyl amyl glycolate | 67634-00-8 | 0.04000 | 2.00 | 0.21 |
| (10% in DPG) ⁴ | | | | |
| Indole (10% in DPG) ⁴ | 120-72-9 | 0.02980 | 1.00 | 0.10 |
| Alpha-Terpineol | 98-55-5 | 0.02830 | 30.00 | 3.13 |
| Diphenyl Oxide | 101-84-8 | 0.02230 | 5.00 | 0.52 |
| L-Citronellol | 7540-51-4 | 0.01830 | 80.00 | 8.34 |
| Beta-Ionone | 14901-07-6 | 0.01690 | 5.00 | 0.52 |
| Alpha-Ionone | 127-41-3 | 0.01440 | 15.00 | 1.56 |
| Dimethyl benzyl carbinyl acetate | 151-05-3 | 0.01390 | 30.00 | 3.13 |
| Geraniol | 106-24-1 | 0.01330 | 40.00 | 4.17 |
| Nerol | n/a | 0.01330 | 20.00 | 2.08 |
| Lilial ® ¹ | 80-54-6 | 0.00444 | 60.00 | 6.25 |
| Gamma-Undecalactone | 104-67-6 | 0.00271 | 15.00 | 1.56 |
| Amyl salicylate | 2050-08-0 | 0.00144 | 25.00 | 2.61 |
| Galaxolide ® | 1222-05-5 | 0.000414 | 20.00 | 2.08 |
| cis-3-Hexenyl salicylate | 65405-77-8 | 0.000246 | 20.00 | 2.08 |
| Ethylene Brassylate | 105-95-3 | 0.00000000313 | 30.00 | 3.13 |
| Styrolyl Acetate ⁵ | n/a | n/a | 20.00 | 2.08 |
| Decenol trans-9 ³ | n/a | n/a | 15.00 | 1.56 |
| Geranium oil ² | n/a | n/a | 15.00 | 1.56 |

| | | |
|---|---|---|
| Total | 959.5 | 100 wt % |

¹ Benzenepropanal, 4-(1,1-dimethylethyl)-α-methyl-.
² Natural oil that is judged to be of moderate volatility for the purposes of calculating levels of the volatile fragrance materials.
³ Proprietary oil that is judged to be of moderate volatile for the purposes of calculating levels of the volatile fragrance materials.
⁴ Fragrance materials added as dilutions in a non-volatilee solvent. For the purposes of calculating the fragrance oil composition actual fragrance materials levels added are used.
⁵ Unknown oil that is judged to be of low volatility for the purposes of calculating levels of the volatile fragrance materials.

### Example 2

### Single Fragrance Material Compositions Containing Fragrance Oils and Substantially Non-Odorous Fragrance Fixatives

Compositions A, C, E, G, I, K, M, O, Q, S, U, W, Y, AA, and CC are examples of compositions according to the present invention, made with single fragrance materials and the substantially non-odorous fragrance fixatives, respectively. In parallel, control Compositions B, D, F, H, J, L, N, P, R, T, V, X, Z, BB, and DD are prepared without a substantially non-odorous fragrance fixative as a control. They are prepared by admixture of the components in Tables 18(a) and 18(b), in the proportions indicated.

TABLE 18(a)
Single Fragrance Material Compositions

| | Single Fragrance Material Composition (wt %¹) | | | | | | | | | | | | | | | |
|---|---|---|---|---|---|---|---|---|---|---|---|---|---|---|---|---|
| Ingredients | A | B | C | D | E | F | G | H | I | J | K | L | M | N | O | P |
| Dimethyl Benzyl | 1 | 1 | — | — | — | — | — | — | 1 | 1 | — | — | — | — | — | — |
| Carbinol | | | | | | | | | | | | | | | | |
| Eugenol | — | | 1 | 1 | — | — | — | — | — | — | 1 | 1 | — | — | — | — |
| Phenylethyl | — | — | — | — | 1 | 1 | — | — | — | — | — | — | 1 | 1 | — | — |
| Alchol | | | | | | | | | | | | | | | | |
| Fragrance A² | — | — | — | — | — | — | 1 | 1 | — | — | — | — | — | — | 1 | 1 |
| Piperonyl butoxide | 2.2 | 0 | 2.0 | 0 | 2.2 | 0 | 0.5-5 | 0 | — | — | — | — | — | — | — | — |
| Poly(PG)monobutyl | — | — | — | — | — | — | — | — | 2.2 | 0 | 2.0 | 0 | 1.8 | 0 | 0.5-5 | 0 |
| ether | | | | | | | | | | | | | | | | |

| | |
|---|---|
| Ethanol | to 100 |

¹Wt % is relative to the total weight of the composition.
²Can be any one of the single fragrance materials of Table 2 or 3.

TABLE 18(b)
Single Fragrance Material Compositions

| | Single Fragrance Material Composition (wt %¹) | | | | | | | | | | | | | |
|---|---|---|---|---|---|---|---|---|---|---|---|---|---|---|
| Ingredients | Q | R | S | T | U | V | W | X | Y | Z | AA | BB | CC | DD |
| Indole | 1 | 1 | — | — | — | — | — | — | 1 | 1 | — | — | — | — |
| Eugenol | — | — | 1 | 1 | — | — | — | — | — | — | 1 | 1 | — | — |
| Dimethyl | — | — | — | — | 1 | 1 | — | — | — | — | — | — | 1 | 1 |
| Benzyl | | | | | | | | | | | | | | |
| Carbinol | | | | | | | | | | | | | | |
| Phenylethyl | — | — | — | — | — | — | 1 | 1 | — | — | — | — | — | — |
| Alchol | | | | | | | | | | | | | | |
| Triglycol | 1.3 | — | 0.9 | — | 1.0 | — | 1.2 | — | — | — | — | — | — | — |

| | |
|---|---|
| Ethanol | To 100 |

¹Wt % is relative to the total weight of the composition.

Composition EE is an example of a composition according to the present invention, made with single fragrance material and the substantially non-odorous fragrance fixative, respectively, that are particularly suited to olfactive evaluation. In parallel, control Composition FF is prepared without a substantially non-odorous fragrance fixative as a control. All the compositions are prepared by admixture of the components in Table 18(c), in the proportions indicated.

TABLE 18(c)
Single Fragrance Material Compositions

| | Single Fragrance Material Composition (wt %) ¹ | |
|---|---|---|
| Ingredients | EE | FF |
| Fragrance A ² | 1-7 | 1-7 |
| Modulator ³ | 1-15.0 | 0.0 |

| | |
|---|---|
| Ethanol | to 100 |

¹ Wt % is relative to the total weight of the composition.
² Can be any one of the fragrance materials disclosed in Tables 2 and 3.
³ Can be any one of the substantially non-odorous fragrance fixatives not already disclosed in Tables 18(a) and 18(b).

Tables 18(d) provides test compositions comprising the a single volatile fragrance material (as disclosed in Table 3) with a substantially non-odorous fragrance fixative (as disclosed in Table 1) that are particularly suited to analytical measurements. All of the compositions are prepared by admixture of the components described in Table 18(d) in the proportions indicated.

TABLE 18(d)
Single Volatile Fragrance Material Compositions

| | Test Composition | Reference Composition |
|---|---|---|
| Ingredients | (wt % ¹) | (wt % ¹) |
| Volatile Fragrance Material ² | 1.0-3.0 | 1.0-3.0 |
| Triethyl citrate | 0.25 to 2.0 | 0.25 to 2.0 |
| Ethanol | 75.0 | 75.0 |
| Fixative ³ | 0.1-10.0 | 0.0 |
| Water | qsp | qsp |
| Total | 100.0 | 100.0 |

¹ Wt % is relative to the total weight of the composition.
² Can be any one of the volatile fragrance material as disclosed in Table 3.
³ Can be any one of the substantially non-odorous fragrance fixative as disclosed in Table 1.

### Example 3

### Compositions Comprising Substantially Non-Odorous Fragrance Fixatives

Composition A1 is an example of a fragrance composition according to the present invention, made with any of the fragrance examples 1-3, 4b, 5b and 18, respectively. Composition B1 is an example of a fragrance composition containing traditional or higher levels of low volatile fragrance materials, made with any of the fragrance examples 4a, 5a, and 7-17, respectively. In parallel, a control composition C1 is prepared by replacing the different substantially non-odorous fragrance fixative by the same amount of deionized water. All of the compositions are prepared by admixture of the components described in Table 19(a) in the proportions indicated.

TABLE 19(a)
Fragrance Composition

| | Fragrance Composition | | |
|---|---|---|---|
| | (wt %) ¹ | | |
| Ingredients | A1 | B1 | C1 |
| Fragrance A1 ² | 2-15 | — | — |
| Fragrance B ³ | — | 2-15 | — |
| Fragrance A1 or B | — | — | 2-15 |
| Ethanol | 60-99.99 | | |
| Butylated Hydroxy | 0-0.07 | | |
| Toluene | | | |
| Modulator A ⁴ | 0.1-20 | 0.1-20 | — |

| | |
|---|---|
| Deionized water | to 100.00 |

¹ Wt % is relative to the total weight of the composition.
² Can be any one of fragrance examples 1-3, 4b, 5b, and 18.
³ Can be any one of fragrance examples 4a, 5a, and 7-17.
⁴ Can be any one of the substantially non-odorous fragrance fixative as disclosed in Table 1.

Tables 19(b) provides test compositions (MOD1 to MOD43) comprising the volatile fragrance formulation of fragrance example 6 (as disclosed in Table 11) with a substantially non-odorous fragrance fixative (as disclosed in Table 1) that are particularly suited to analytical measurements. All of the compositions are prepared by admixture of the components described in Table 19(b) in the proportions indicated.

TABLE 19(b)
Compositions comprising fragrance with 10 Volatile
Fragrance Materials

| | Test composition | Reference composition |
|---|---|---|
| | (wt %) | (wt %) |
| Ingredients | MOD 1 to 43 | REF |
| Fragrance A ² | 7.0 | 7.0 |
| Triethyl citrate | 0.25 to 1.0 | 0.25 to 1.0 |
| Ethanol | 75.0 | 75.0 |
| Fixative ³ | 15.0 | 0.0 |
| Water | qsp | qsp |
| Total | 100.0 | 100.0 |

¹ Wt % is relative to the total weight of the composition.
² Fragrance Example 6 (as disclosed in Table 11).
³ Can be any one of the substantially non-odorous fragrance fixative no. 1- 3, 6-10, 12-13, 15-19, 21, 26-28, 32, 47, 49-50, 52, 63, 84, 101, 106, 121, 128, 130, 138, 142, 143, 144, 151, 152, 159, 173, 180, and 189 as disclosed in Table 1.

Tables 19(c) provides test compositions comprising the volatile fragrance formulation of fragrance example 6 (as disclosed in Table 11) with a substantially non-odorous fragrance fixative (as disclosed in Table 1) that are particularly suited to analytical measurements. All of the compositions are prepared by admixture of the components described in Table 19(c) in the proportions indicated.

TABLE 19(c)
Compositions comprising fragrance with 10 Volatile
Fragrance Materials

| | Test composition | Reference composition |
|---|---|---|
| Ingredients | (wt % ¹) | (wt % ¹) |
| Fragrance A ² | 0.4-7.0 | 0.4-7.0 |
| Triethyl citrate | 0.25 to 2.0 | 0.25 to 2.0 |
| Ethanol | 75.0 | 75.0 |
| Fixative ³ | 1-15.0 | 0.0 |
| Water | qsp | qsp |
| Total | 100.0 | 100.0 |

¹ Wt % is relative to the total weight of the composition.
² Fragrance Example 6 (as disclosed in Table 11).
³ Can be any one of the substantially non-odorous fragrance fixative no. 4-5, 11, 14, 20, 22-25, 29-31, 33-46, 48, 51, 53-62, 64-83, 85-100, 102-105, 107-120, 122-127, 129, 131-137, 139-141, 145-150, 153-158, 160-172, 174-179, 181-188, and 190 as disclosed in Table 1.

### Example 4

### Exemplary Product Compositions

Compositions I, II, III and IV are examples of body spray compositions according to the present invention. They are prepared by admixture of the components described in Table 20, in the proportions indicated.

TABLE 20
Body Spray Compositions

| | CAS | Compositions (wt % ¹) | | | |
|---|---|---|---|---|---|
| Ingredients | Number | I | II | III | IV |
| Denatured Ethanol | 64-17-5 | 39.70 | 59.45 | 39.70 | 39.70 |
| Water | 7732-18-5 | — | 0.75 | — | — |
| Dipropylene Glycol | 25265-71-8 | 15.00 | — | 15.00 | 15.00 |
| Isopropyl Myristate | 110-27-0 | 1.00 | — | 1.00 | 1.00 |
| Zinc | 127-82-2 | 0.50 | — | 0.50 | 0.50 |
| Phenosulphonate | | | | | |
| Cavasol ® W7 | 128446-36-6 | — | 1.00 | — | — |
| methylated | | | | | |
| Beta-cyclodextrin | | | | | |
| Fragrance ² | — | 1.20 | 1.20 | 1.20 | 1.20 |
| Fragrance Fixative ³ | — | 2.60 | 2.60 | 2.60 | 2.60 |
| Propane | 74-98-6 | 4.86 | — | 4.86 | 4.86 |
| Isobutane | 72-28-5 | 27.14 | — | 27.14 | 27.14 |
| 1,1-Difluoroethane | 75-37-6 | 8.00 | 35.00 | 8.00 | 8.00 |
| (HFC-152a) | | | | | |

| | | | | |
|---|---|---|---|---|
| Total | 100.00 | 100.00 | 100.00 | 100.00 |

¹ wt % relative to the total weight of the composition.
² Can be any one of Fragrances Examples 1, 2, 3, 4a, 4b, 5a, 5b, and 7-17.
³ Can be any one of the substantially non-odorous fragrance fixatives disclosed in Table 1.

Composition V, VI and VII are examples of body lotion compositions according to the present invention. They are prepared by admixture of the components as described in Table 21, in the proportions indicated.

TABLE 21
Body Lotion Composition

| | CAS | Compositions (wt % ¹) | | |
|---|---|---|---|---|
| Ingredients | Number | V | VI | VII |
| Water | 7732-18-5 | qsp | qsp | qsp |
| | | 100% | 100% | 100% |
| Trilon ® B | 64-02-8 | 0.05 | 0.05 | 0.05 |
| Carbopol ® ETD 2050 | 9003-01-4 | 0.2 | 0.2 | 0.2 |
| Pemulen ™ TR1 | 9063-87-0 | 0.2 | 0.2 | 0.2 |
| Nexbase ® 2008 | 68037-01-4 | 8 | 8 | 8 |
| Silicone V100 | 63148-62-9 | 6 | 6 | 6 |
| Fragrance Fixative ³ | — | 3 | 3 | 3 |
| Tris Amino ™ Ultra | 102-71-6 | 0.4 | 0.4 | 0.4 |
| Pur | | | | |
| Fragrance ² | — | 3 | 3 | 3 |
| Preservatives | — | qs | qs | qs |

| | | | |
|---|---|---|---|
| Total | 100.00 | 100.00 | 100.00 |

¹ wt % relative to the total weight of the composition.
² Can be any one of the Fragrances Examples 1, 2, 3, 4a, 4b, 5a, 5b, and 7-17.
³ Can be any one of the substantially non-odorous fragrance fixatives disclosed in Table 1.

### Example 5

### Olfactive Test Results

Compositions disclosed in Tables 18(a)-18(c), and 19(a) are applied to glass slides in accordance with the protocol described in the Method Section and a panel of 6-11 experienced panelists evaluated the perceived fragrance profile at initial time 0, then at various time points, typically 1 hour, 2 hours, 3 hours, 4 hours and 6 hours post application. Panelists are asked to score the compositions for the longevity on a scale of 0 to 5, wherein 0 represents a no fragrance is detected and 5 represents a very strong fragrance intensity is detected; and for fragrance profile fidelity on a scale of 0 to 3 wherein 0 represents not detectable and 3 represents it being the dominant character. The results of the panelists are then averaged and discussed below.

(a) Effects of the Substantially Non-Odorous Fragrance Fixatives on Single Fragrance Material Compositions

FIG. 1 shows the fragrance intensity profile of Composition A as evaluated by 10 panelists, which comprises the substantially non-odorous fragrance fixative Piperonyl butoxide, on the single fragrance material, Dimethyl Benzyl Carbinol. Addition of the fixative maintains the intensity of the fragrance material whilst the control, Composition B, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 1 hour (p=0.0061) at 95% significance level (i.e., p<0.05).

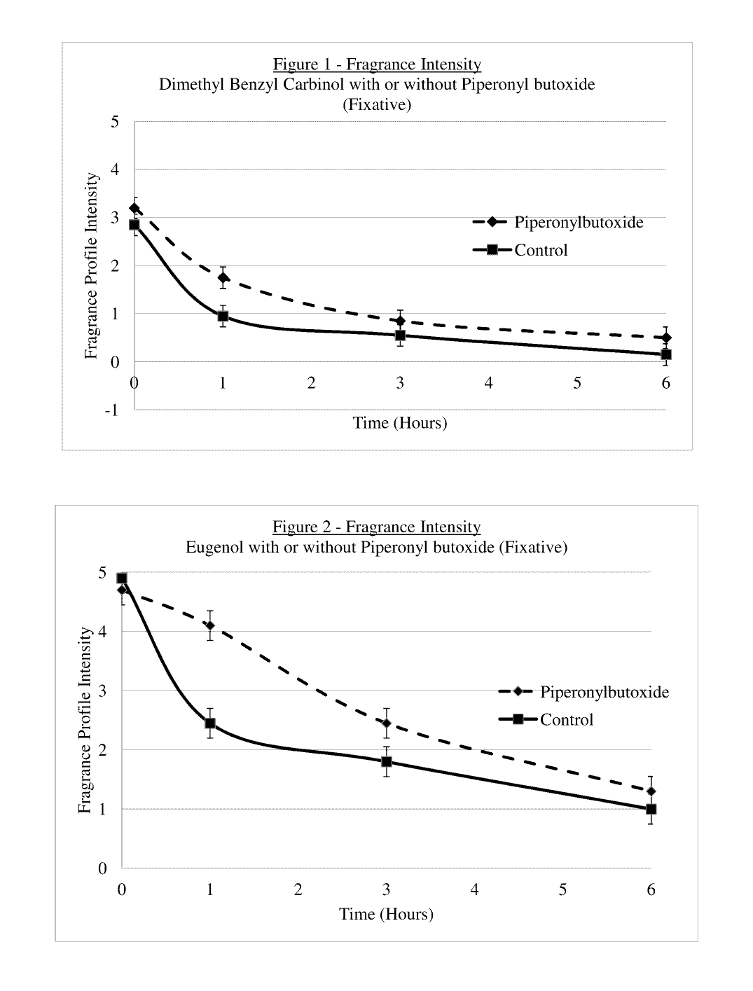

FIG. 2 shows the fragrance intensity profile of Composition C as evaluated by 10 panelists, which comprises the substantially non-odorous fragrance fixative Piperonyl butoxide, on the single fragrance material, Eugenol. Addition of the fixative (Piperonyl butoxide) maintains the intensity of the fragrance material whilst the control, Composition D, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 1 hour (p<0.0001) and at 3 hours (p=0.0231) at 95% significance level (i.e., p<0.05).

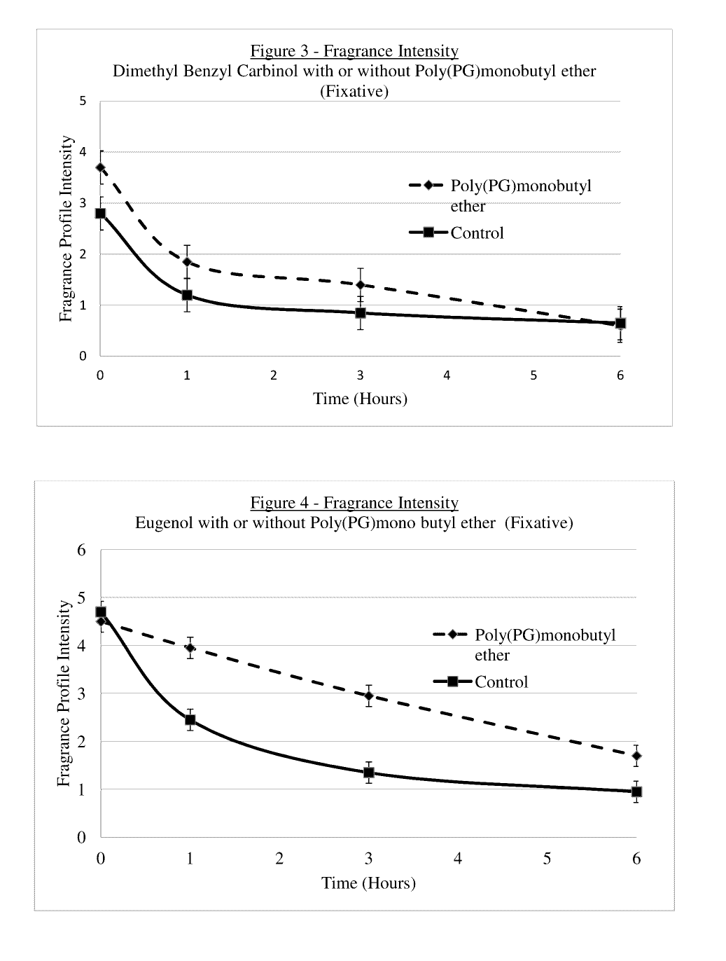

FIG. 3 shows the fragrance intensity profile of Composition I as evaluated by 10 panelists, which comprises the substantially non-odorous fragrance fixative Poly(PG)monobutyl ether, on the single fragrance material, Dimethyl Benzyl Carbinol. Addition of the fixative (Poly(PG)monobutyl ether) maintains the intensity of the fragrance material whilst the control, Composition J, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 0 hours (p=0.0060) and 1 hour (p=0.0443) at 95% significance level (i.e., p<0.05) and at 3 hours (p=0.0873) at 90% significance (i.e., p<0.1).

FIG. 4 shows the fragrance intensity profile of Composition K as evaluated by 10 panelists, which comprises the substantially non-odorous fragrance fixative Poly(PG)monobutyl ether, on the single fragrance material, Eugenol. Addition of the fixative (Poly(PG)monobutyl ether) maintains the intensity of the fragrance material whilst the control, Composition L, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 1 hour (p<0.0001), at 3 hours (p<0.0001) and at 6 hours (p=0.0067) at 95% significance level (i.e., p<0.05).

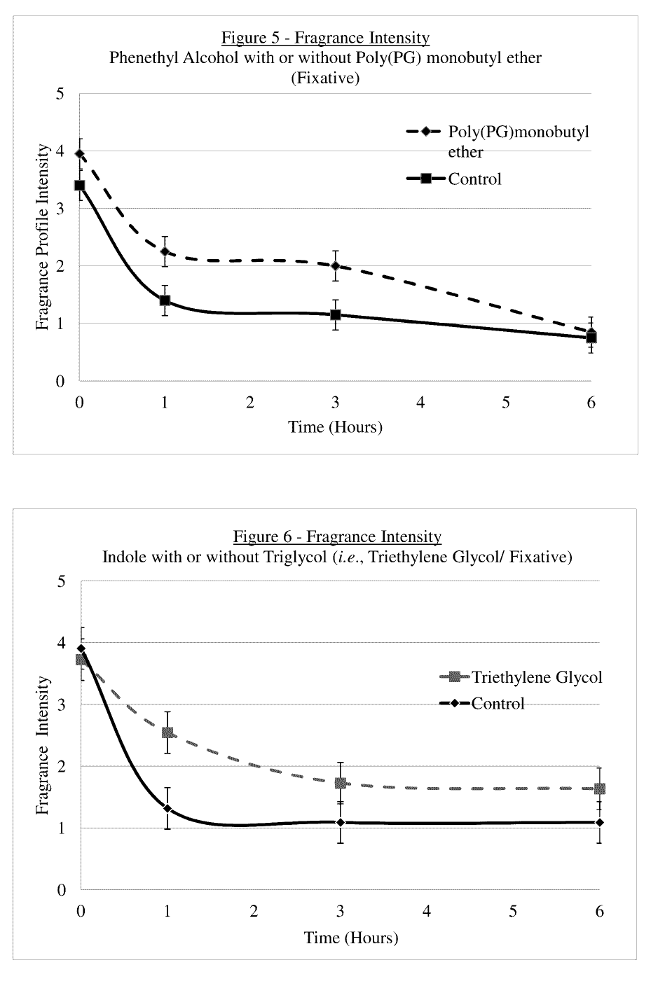

FIG. 5 shows the fragrance intensity profile of Composition M as evaluated by 10 panelists, which comprises the substantially non-odorous fragrance fixative Poly(PG)monobutyl ether, on the single fragrance material, phenethyl alcohol (PEA). Addition of the fixative (Poly(PG)monobutyl ether) maintains the intensity of the fragrance material whilst the control, Composition N, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 0 hours (p=0.0530) at 90% significance level (i.e., p<0.1) and at 1 hour (p<0.0034) and at 3 hours (p<0.0034) at 95% significance level (i.e., p<0.05).

FIG. 6 shows the fragrance intensity profile of Composition Q as evaluated by 11 panelists, which comprises the substantially non-odorous fragrance fixative Triglycol, on the single fragrance material Indole. Addition of the fixative (Triglycol) maintains the intensity of the fragrance material whilst the control, Composition R, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 1 hour (p<0.0014) at 95% significance level (i.e., p<0.05).

FIG. 7 shows the fragrance intensity profile of Composition S as evaluated by 11 panelists, which comprises the substantially non-odorous fragrance fixative Triglycol, on the single fragrance material Eugenol. Addition of the fixative (Triglycol) maintains the intensity of the fragrance material whilst the control, Composition T, in the absence of the substantially non-odorous fragrance fixative, drops in fragrance intensity profile over the 6 hours. The substantially non-odorous fragrance fixative acts to maintain the continued evaporation over time of the fragrance material. Statistical analysis using the Tukey correction for multiple comparisons confirms the statistically significant difference at 1 hour (p<0.0144) at 95% significance level (i.e., p<0.05).

(b) Effects of Substantially Non-Odorous Fragrance Fixatives on the Fragrance Profile Longevity of Compositions Having Reduced Levels of Low Volatile Fragrance Materials (Between 10 to 30 wt % Relative to the Total Weight of the Fragrance Component) Vs. Compositions Having Traditional Levels of Low Volatile Fragrance Materials (Greater than 30 wt % Relative to the Total Weight of the Fragrance Component) and No Substantially Non-Odorous Fragrance Fixative

Panelists are asked to score the compositions for the intensity of the fragrance on a scale of 0 to 5, wherein 0 represents no fragrance intensity is detected and 5 represents a very strong fragrance intensity is detected. The results of the panel test are then averaged. The results show the effect of the substantially non-odorous fragrance fixative and reduced levels of low volatile fragrance materials for any one of the inventive Compositions A1 on fragrance profile longevity versus control Compositions C1 in the absence of the substantially non-odorous fragrance fixatives. Alternatively, the results show the effect of the substantially non-odorous fragrance fixative and reduced levels of low volatile fragrance materials for any one of the inventive Compositions A1 on fragrance profile longevity versus traditional Compositions B1 in the presence of the substantially non-odorous fragrance fixative.

Fragrance profile longevity, particularly intensity of the characters attributable to the volatile fragrance materials, are maintained for up to at least 6 hours in the presence of the substantially non-odorous fragrance fixative whilst it drops in the absence of the substantially non-odorous fragrance fixative.

(c) Effects of the Substantially Non-Odorous Fragrance Fixatives on the Fragrance Profile Fidelity of Compositions Having Reduced Levels of Low Volatile Fragrance Materials (Between 10 to 30 wt % Relative to the Total Weight of the Fragrance Component) Vs. Compositions Having Traditional Levels of Low Volatile Fragrance Materials (Greater than 30 wt % Relative to the Total Weight of the Fragrance Component) and No Substantially Non-Odorous Fragrance Fixative

Panelists are also asked to score the composition for the fragrance profile fidelity. In particular, the panelists are asked to score the dominance of the floral character attributable to the volatile fragrance materials on a scale of 0 to 3 wherein 0 represents not detectable and 3 represents it being the dominant character. The results of the panel test are then averaged. The results show the effect of the substantially non-odorous fragrance fixative for the inventive Compositions A1 on fragrance profile fidelity versus control Compositions C1 in the absence of the substantially non-odorous fragrance fixative.

Fragrance profile fidelity are maintained by the substantially non-odorous fragrance fixative over time for up to 6 hours in the presence of the substantially non-odorous fragrance fixative whilst it drops in the absence of the substantially non-odorous fragrance fixative (data not shown).

### Example 6

### Analytical Evaporation Test Results

Using the analytical evaporation Test Method 3, it is possible to measure the amount of a volatile fragrance material or each component of a perfume mixture that remains as the fragrance mixture evaporates. Test compositions may comprise any one of the volatile fragrance material as disclosed in Table 3 and a substantially non-odorous fragrance fixative, as disclosed in Table 1. Alternatively, test compositions may comprise a mixture of 10 volatile perfume materials, as disclosed in Table 11 (Fragrance Example 6), and a substantially non-odorous fragrance fixative, as disclosed in Table 1. Examples of suitable test compositions include the compositions disclosed in Table 18(d), and Compositions MOD1 to MOD43 in Table 19(b) and Compositions in Table 19(c). The test compositions are introduced in the aluminum containers at the set temperature for pre-determined periods of time in accordance with the protocol described in Test Method 3.

For Compositions MOD1 to MOD43, indole is one of the components of the 10 PRMs mixture of Table 11. Control compositions containing the full 10 PRMs, or one component (e.g., indole), without the substantially non-odorous fragrance fixative are run alongside the test compositions. The average profile for the control composition is plotted against the individual profile for the indole component from the test composition containing the 10 PRMs mixture of Table 11 with the substantially non-odorous fragrance fixatives. The error associated with the method is determined by running replicate evaporation experiments on the control composition. An average evaporation profile of the control composition as well as the 95% confidence interval at each time point are calculated from the replicates.

It is useful to consider the difference (Δ) in the % of remaining fragrance material between each of the test composition (MOD) and their respective control composition (REF) at each experimental time points (e.g., 30 mins, 60 mins and 180 mins) to determine the effect of the substantially non-odorous fragrance fixative on the volatile PRMs in a mixture. The difference (Δ) in the % of remaining of a given fragrance material is calculated as follows:

Δ=% remaining of given fragrance material in test composition (MOD)−% remaining of same fragrance material in control composition (REF)

The difference (Δ) can then be plotted (data not shown) for each of the perfume materials in the mixture at each of the time points. For ease of reference, the applicant has summarize the effect of the substantially non-odorous fragrance fixative on only one volatile fragrance component (e.g., indole) of the mixture, to serve as a representative of all of the volatile fragrance materials.

(a) Effects of Tergitol® 15-S-7 on Composition Having Volatile Fragrance Materials

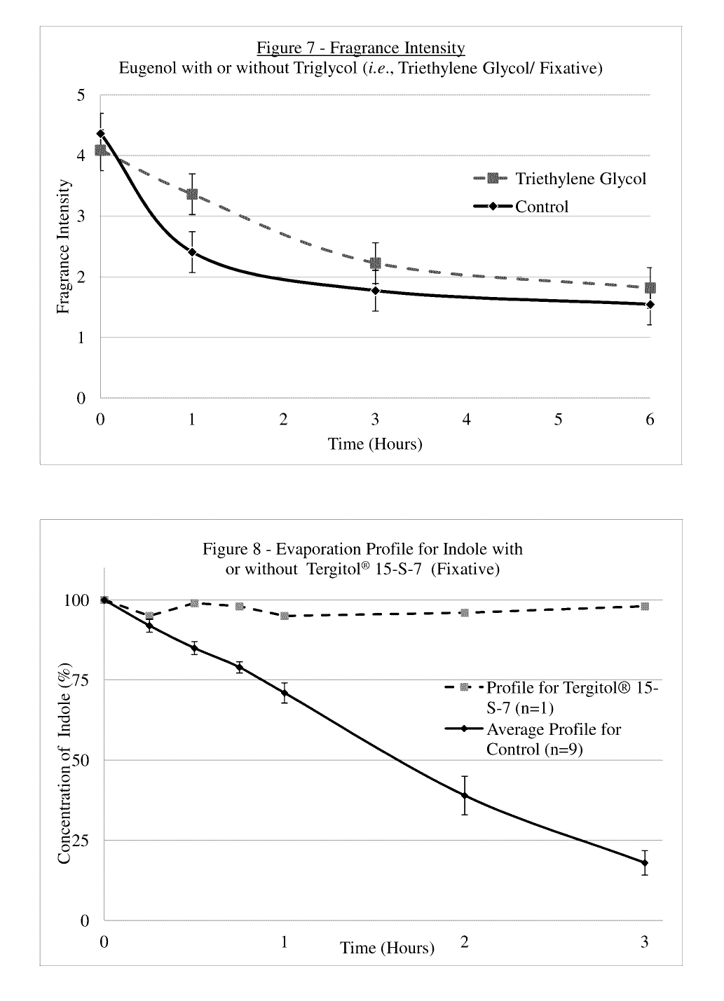

FIG. 8 shows the effect of the substantially non-odorous fragrance fixative Tergitol® 15-S-7 on the evaporation profile for a representative component (i.e., indole) of the test composition (MOD1). With reference to FIG. 8, indole has a difference (Δ) of 14% after 30 mins, 24% after 60 mins, and 80% after 3 hours. Addition of the Tergitol® 15-S-7 in the test composition (MOD1) maintains the concentration of the volatile fragrance material indole from 0 hour up to 3 hours whilst the control composition (REF), in the absence of Tergitol® 15-S-7, drops in fragrance concentration over the 3 hours. Thus, Tergitol® 15-S-7 acts to maintain the continued evaporation of the volatile fragrance material over time. Similar results are observed for the other volatile fragrance materials in the mixture (data not shown).

(b) Effects of PPG-7-Buteth-10 on Composition Having Volatile Fragrance Materials

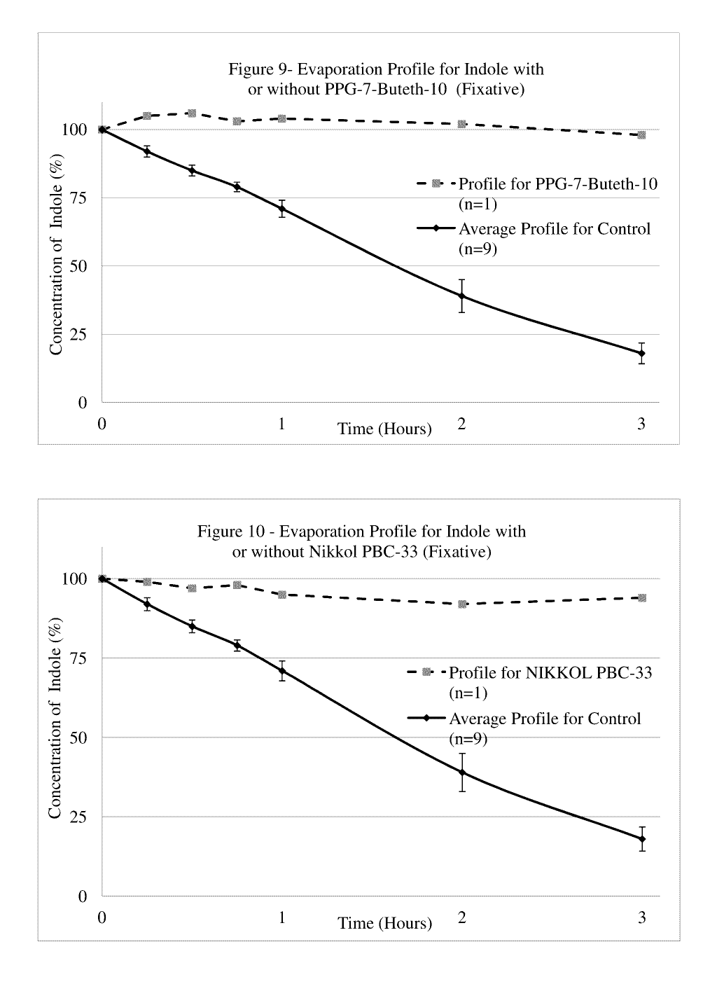

FIG. 9 shows the effect of the substantially non-odorous fragrance fixative PPG-7-Buteth-10 on the evaporation profile for a representative component (i.e., indole) of the test composition (MOD2). With reference to FIG. 9, indole has a difference (Δ) of 21% after 30 mins, 33% after 60 mins, and 80% after 3 hours. Addition of the Tergitol® in the test composition (MOD2) maintains the concentration of the volatile fragrance material indole from 0 hour up to 3 hours whilst the control composition (REF), in the absence of PPG-7-Buteth-10, drops in fragrance concentration over the 3 hours. Thus, PPG-7-Buteth-10 acts to maintain the continued evaporation of the volatile fragrance material over time. Similar results are observed for the other volatile fragrance materials in the mixture (data not shown).

(c) Effects of Nikkol PBC-33 on Composition Having Volatile Fragrance Materials

FIG. 10 shows the effect of the substantially non-odorous fragrance fixative Nikkol PBC-33 on the evaporation profile for a representative component (i.e., indole) of the test composition (MOD3). With reference to FIG. 10, indole has a difference (Δ) of 12% after 30 mins, 24% after 60 mins, and 76% after 3 hours. Addition of the Nikkol PBC-33 in the test composition (MOD3) maintains the concentration of the volatile fragrance material indole from 0 hour up to 3 hours whilst the control composition (REF), in the absence of Nikkol PBC-33, drops in fragrance concentration over the 3 hours. Thus, Nikkol PBC-33 acts to maintain the continued evaporation of the volatile fragrance material over time. Similar results are observed for the other volatile fragrance materials in the mixture (data not shown).

(d) Effects of Neodol 45-7 Alcohol Ethoxylate on Composition Having Volatile Fragrance Materials

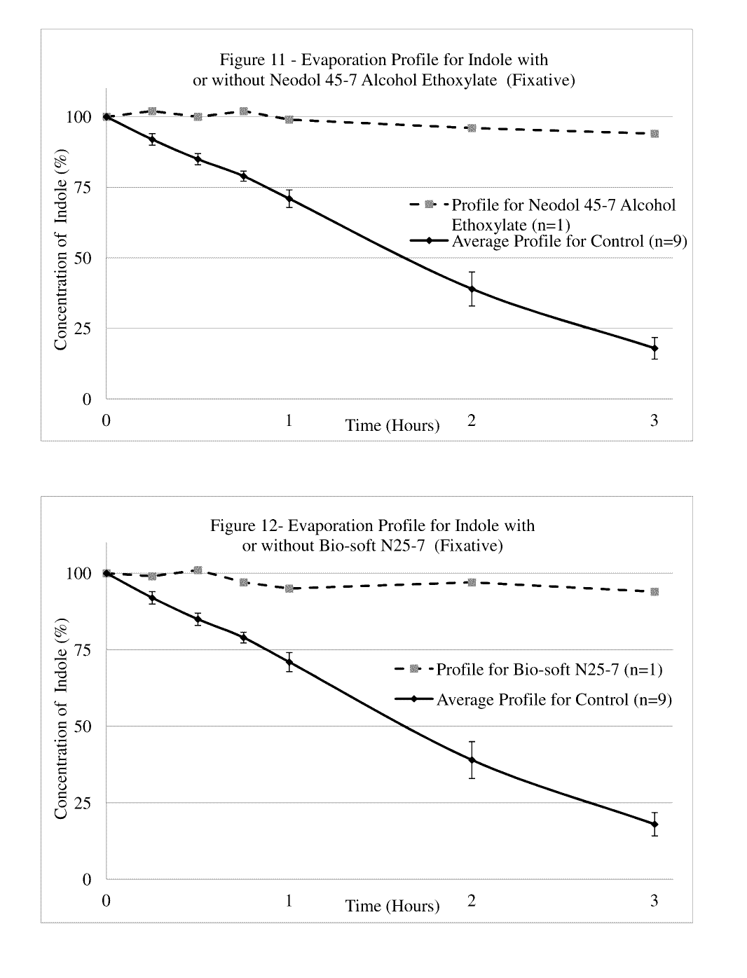

FIG. 11 shows the effect of the substantially non-odorous fragrance fixative Neodol 45-7 Alcohol Ethoxylate on the evaporation profile for a representative component (i.e., indole) of the test composition (MOD4). With reference to FIG. 11, indole has a difference (Δ) of 15% after 30 mins, 28% after 60 mins, and 76% after 3 hours. Addition of the Neodol 45-7 Alcohol Ethoxylate in the test composition (MOD4) maintains the concentration of the volatile fragrance material indole from 0 hour up to 3 hours whilst the control composition (REF), in the absence of Neodol 45-7 Alcohol Ethoxylate, drops in fragrance concentration over the 3 hours. Thus, Neodol 45-7 Alcohol Ethoxylate acts to maintain the continued evaporation of the volatile fragrance material over time. Similar results are observed for the other volatile fragrance materials in the mixture (data not shown).

(e) Effects of Bio-Soft N25-7 on Composition Having Volatile Fragrance Materials

FIG. 12 shows the effect of the substantially non-odorous fragrance fixative Bio-soft N25-7 on the evaporation profile for a representative component (i.e., indole) of the test composition (MOD5). With reference to FIG. 12, indole has a difference (Δ) of 16% after 30 mins, 24% after 60 mins, and 76% after 3 hours. Addition of the Bio-soft N25-7 in the test composition (MOD5) maintains the concentration of the volatile fragrance material indole from 0 hour up to 3 hours whilst the control composition (REF), in the absence of Bio-soft N25-7, drops in fragrance concentration over the 3 hours. Thus, Bio-soft N25-7 acts to maintain the continued evaporation of the volatile fragrance material over time. Similar results are observed for the other volatile fragrance materials in the mixture (data not shown).

(f) Effects of Bio-Soft N23-6.5 on Composition Having Volatile Fragrance Materials

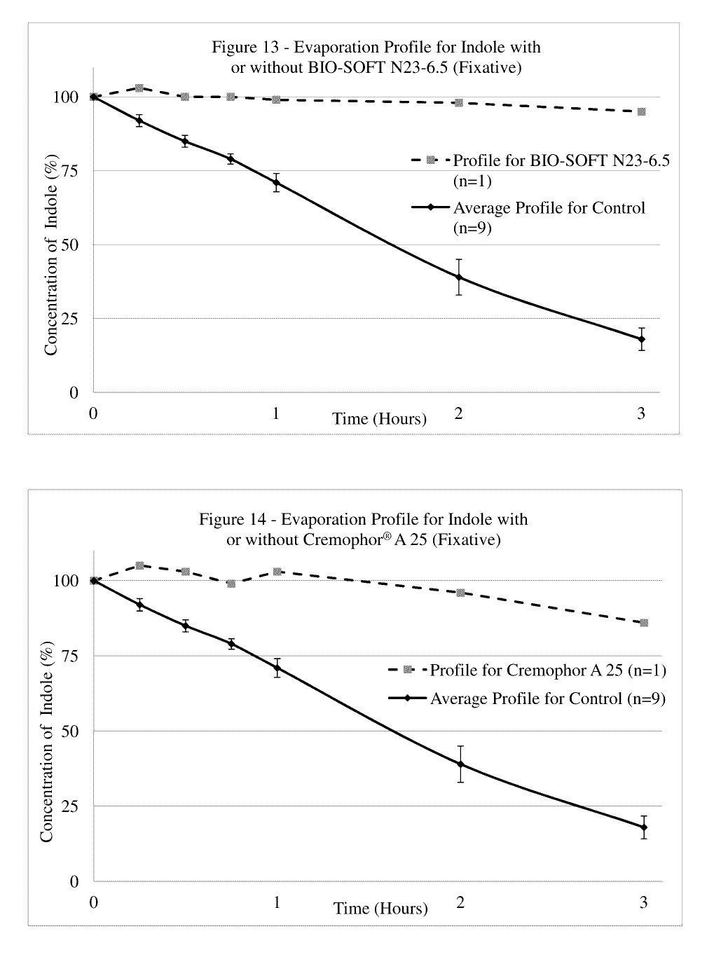

FIG. 13 shows the effect of the substantially non-odorous fragrance fixative Bio-soft N23-6.5 on the evaporation profile for a representative component (i.e., indole) of the test composition (MOD6). With reference to FIG. 13, indole has a difference (Δ) of 15% after 30 mins, 28% after 60 mins, and 77% after 3 hours. Addition of the Bio-soft N23-6.5 in the test composition (MOD6) maintains the concentration of the volatile fragrance material indole from 0 hour up to 3 hours whilst the control composition (REF), in the absence of Bio-soft N23-6.5, drops in fragrance concentration over the 3 hours. Thus, Bio-soft N23-6.5 acts to maintain the continued evaporation of the volatile fragrance material over time. Similar results are observed for the other volatile fragrance materials in the mixture (data not shown).

(g) Effects of Cremophor® A 25 on Composition Having Volatile Fragrance Materials

FIG. 14 shows the effect of the substantially non-odorous fragrance fixative Cremophor® A 25 on the evaporation profile for a representative component (i.e., indole) of the test composition (MOD7). With reference to FIG. 14, indole has a difference (Δ) of 18% after 30 mins, 32% after 60 mins, and 68% after 3 hours. Addition of the Cremophor® A 25 in the test composition (MOD7) maintains the concentration of the volatile fragrance material indole from 0 hour up to 3 hours whilst the control composition (REF), in the absence of Cremophor® A 25, drops in fragrance concentration over the 3 hours. Thus, Cremophor® A 25 acts to maintain the continued evaporation of the volatile fragrance material over time. Similar results are observed for the other volatile fragrance materials in the mixture (data not shown).

(h) Effects of Bio-Soft N91-8 on Composition Having Volatile Fragrance Materials

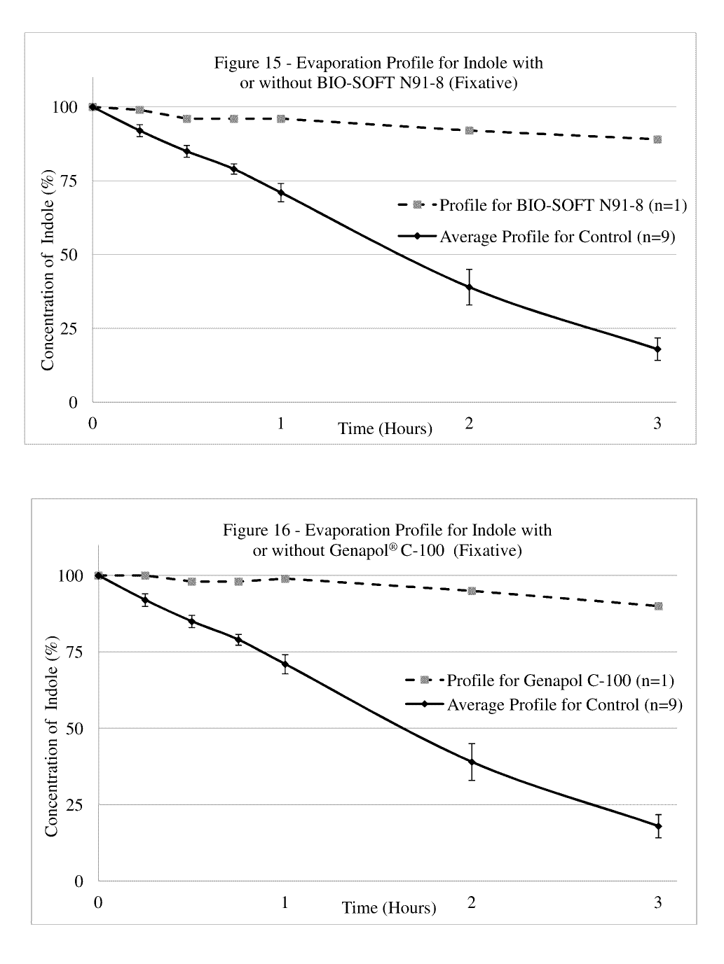

FIG. 15 shows the effect of the substantially non-odorous fragrance fixative Bio-soft N91-8 on the evaporation profile for a representative component (i.e., indole) of the test composition (MOD8). With reference to FIG. 15, indole has a difference (Δ) of 11% after 30 mins, 25% after 60 mins, and 71% after 3 hours. Addition of the Bio-soft N91-8 in the test composition (MOD8) maintains the concentration of the volatile fragrance material indole from 0 hour up to 3 hours whilst the control composition (REF), in the absence of Bio-soft N91-8, drops in fragrance concentration over the 3 hours. Thus, Bio-soft N91-8 acts to maintain the continued evaporation of the volatile fragrance material over time. Similar results are observed for the other volatile fragrance materials in the mixture (data not shown).

(i) Effects of Genapol® C-100 on Composition Having Volatile Fragrance Materials

FIG. 16 shows the effect of the substantially non-odorous fragrance fixative Genapol® C-100 on the evaporation profile for a representative component (i.e., indole) of the test composition (MOD9). With reference to FIG. 16, indole has a difference (Δ) of 13% after 30 mins, 28% after 60 mins, and 72% after 3 hours. Addition of the Genapol® C-100 in the test composition (MOD9) maintains the concentration of the volatile fragrance material indole from 0 hour up to 3 hours whilst the control composition (REF), in the absence of Genapol® C-100, drops in fragrance concentration over the 3 hours. Thus, Genapol® C-100 acts to maintain the continued evaporation of the volatile fragrance material over time. Similar results are observed for the other volatile fragrance materials in the mixture (data not shown).

(j) Effects of Rhodasurf® LA 30 on Composition Having Volatile Fragrance Materials

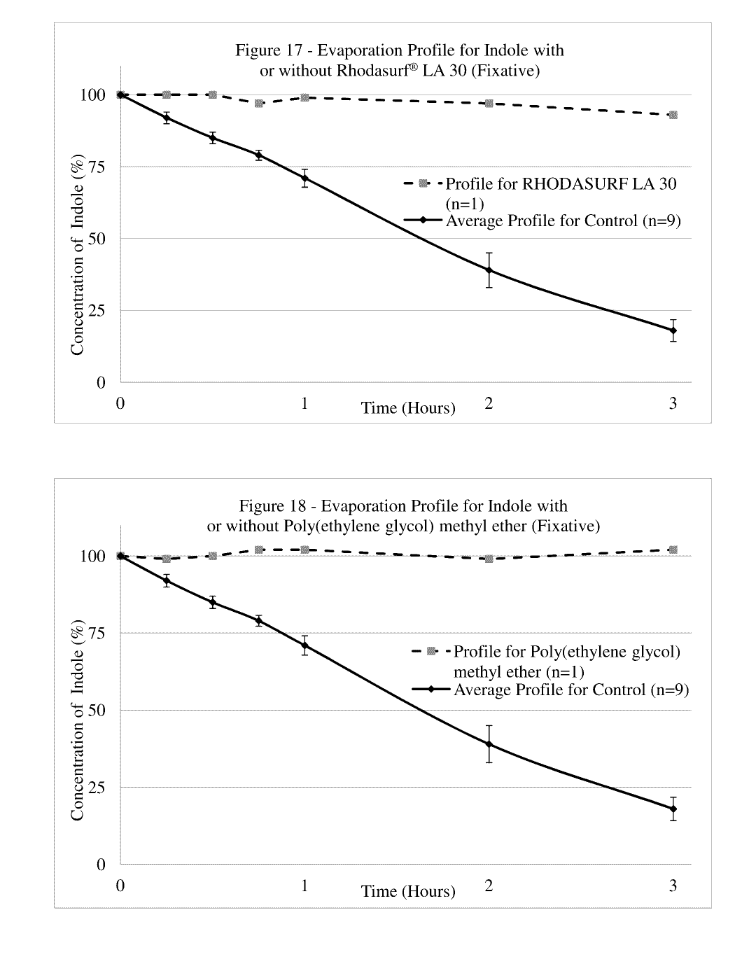

FIG. 17 shows the effect of the substantially non-odorous fragrance fixative Rhodasurf® LA 30 on the evaporation profile for a representative component (i.e., indole) of the test composition (MOD10). With reference to FIG. 17, indole has a difference (Δ) of 15% after 30 mins, 28% after 60 mins, and 75% after 3 hours. Addition of the Rhodasurf® LA 30 in the test composition (MOD10) maintains the concentration of the volatile fragrance material indole from 0 hour up to 3 hours whilst the control composition (REF), in the absence of Rhodasurf® LA 30, drops in fragrance concentration over the 3 hours. Thus, Rhodasurf® LA 30 acts to maintain the continued evaporation of the volatile fragrance material over time. Similar results are observed for the other volatile fragrance materials in the mixture (data not shown).

(k) Effects of Poly(Ethylene Glycol) Methyl Ether on Composition Having Volatile Fragrance Materials

FIG. 18 shows the effect of the substantially non-odorous fragrance fixative Poly(ethylene glycol) methyl ether on the evaporation profile for a representative component (i.e., indole) of the test composition (MOD11). With reference to FIG. 18, indole has a difference (Δ) of 15% after 30 mins, 31% after 60 mins, and 84% after 3 hours. Addition of the Poly(ethylene glycol) methyl ether in the test composition (MOD11) maintains the concentration of the volatile fragrance material indole from 0 hour up to 3 hours whilst the control composition (REF), in the absence of Poly(ethylene glycol) methyl ether, drops in fragrance concentration over the 3 hours. Thus, Poly(ethylene glycol) methyl ether acts to maintain the continued evaporation of the volatile fragrance material over time. Similar results are observed for the other volatile fragrance materials in the mixture (data not shown).

(l) Effects of Arlamol™ PS11E on Composition Having Volatile Fragrance Materials

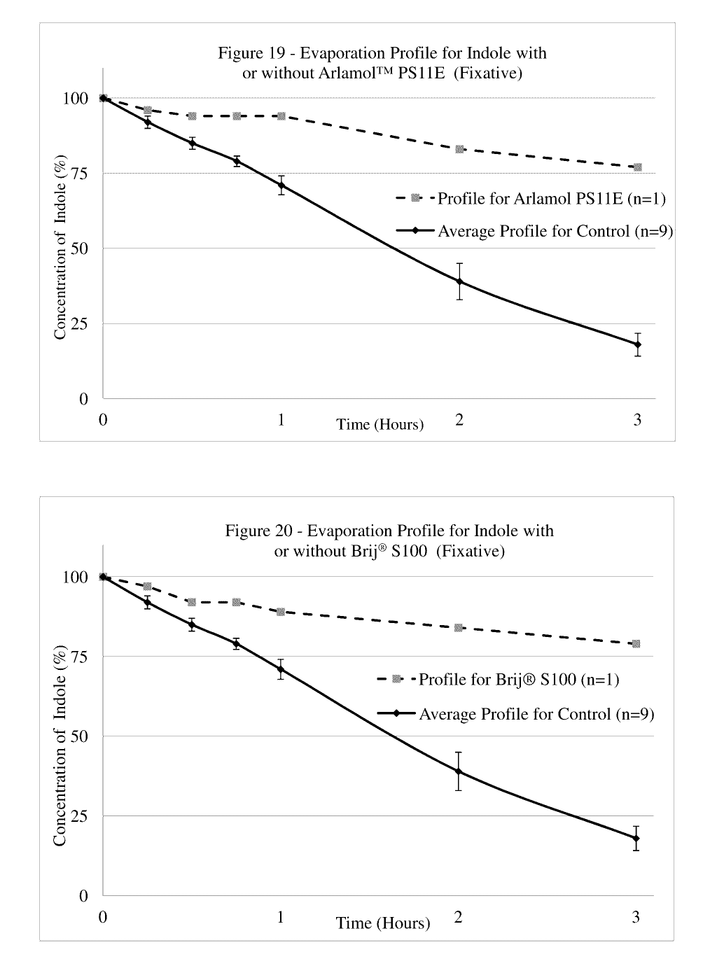

FIG. 19 shows the effect of the substantially non-odorous fragrance fixative Arlamol™ PS11E on the evaporation profile for a representative component (i.e., indole) of the test composition (MOD12). With reference to FIG. 19, indole has a difference (Δ) of 9% after 30 mins, 23% after 60 mins, and 59% after 3 hours. Addition of the Arlamol™ PS11E in the test composition (MOD12) maintains the concentration of the volatile fragrance material indole from 0 hour up to 3 hours whilst the control composition (REF), in the absence of Arlamol™ PS11E, drops in fragrance concentration over the 3 hours. Thus, Arlamol™ PS11E acts to maintain the continued evaporation of the volatile fragrance material over time. Similar results are observed for the other volatile fragrance materials in the mixture (data not shown).

(m) Effects of Brij® S100 on Composition Having Volatile Fragrance Materials

FIG. 20 shows the effect of the substantially non-odorous fragrance fixative Brij® S100 on the evaporation profile for a representative component (i.e., indole) of the test composition (MOD13). With reference to FIG. 20, indole has a difference (Δ) of 7% after 30 mins, 18% after 60 mins, and 61% after 3 hours. Addition of the Brij® S100 in the test composition (MOD13) maintains the concentration of the volatile fragrance material indole from 0 hour up to 3 hours whilst the control composition (REF), in the absence of Brij® S100, drops in fragrance concentration over the 3 hours. Thus, Brij® S100 acts to maintain the continued evaporation of the volatile fragrance material over time. Similar results are observed for the other volatile fragrance materials in the mixture (data not shown).
(n) Effects of Brij® C-10 on Composition Having Volatile Fragrance Materials

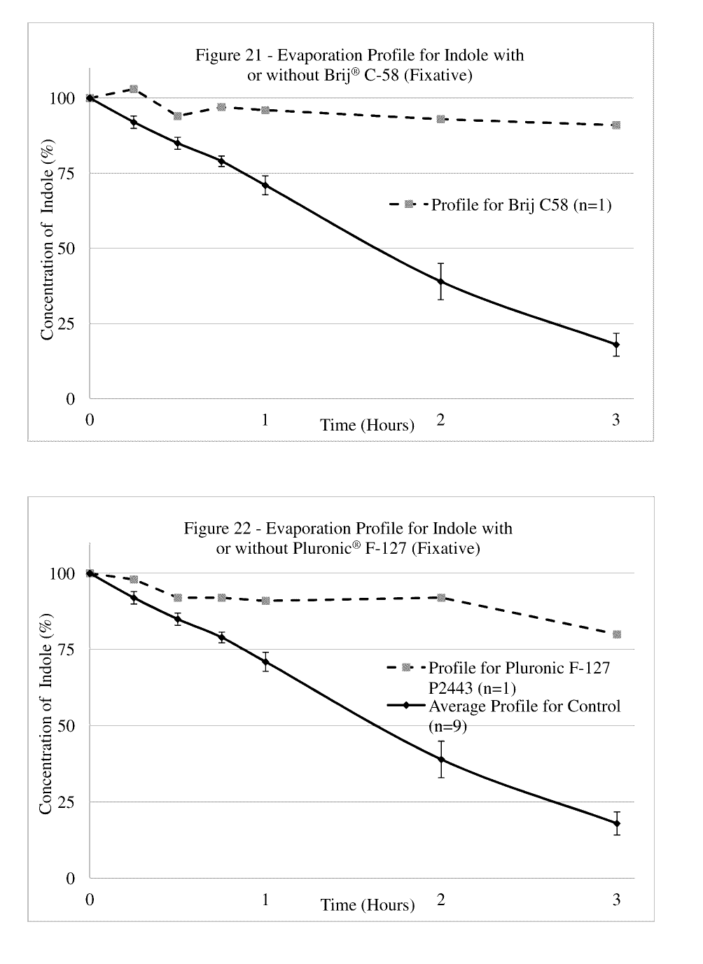

FIG. 21 shows the effect of the substantially non-odorous fragrance fixative Brij® C-58 on the evaporation profile for a representative component (i.e., indole) of the test composition (MOD14). With reference to FIG. 21, indole has a difference (Δ) of 9% after 30 mins, 25% after 60 mins, and 73% after 3 hours. Addition of the Brij® C-58 in the test composition (MOD14) maintains the concentration of the volatile fragrance material indole from 0 hour up to 3 hours whilst the control composition (REF), in the absence of Brij® C-58, drops in fragrance concentration over the 3 hours. Thus, Brij® C-58 acts to maintain the continued evaporation of the volatile fragrance material over time. Similar results are observed for the other volatile fragrance materials in the mixture (data not shown).
(o) Effects of Pluronic® F-127 on Composition Having Volatile Fragrance Materials

FIG. 22 shows the effect of the substantially non-odorous fragrance fixative Pluronic® F-127 on the evaporation profile for a representative component (i.e., indole) of the test composition (MOD15). With reference to FIG. 22, indole has a difference (Δ) of 7% after 30 mins, 20% after 60 mins, and 62% after 3 hours. Addition of the Pluronic® F-127 in the test composition (MOD15) maintains the concentration of the volatile fragrance material indole from 0 hour up to 3 hours whilst the control composition (REF), in the absence of Pluronic® F-127, drops in fragrance concentration over the 3 hours. Thus, Pluronic® F-127 acts to maintain the continued evaporation of the volatile fragrance material over time. Similar results are observed for the other volatile fragrance materials in the mixture (data not shown).

(p) Effects of Bio-Soft N1-5 on Composition Having Volatile Fragrance Materials

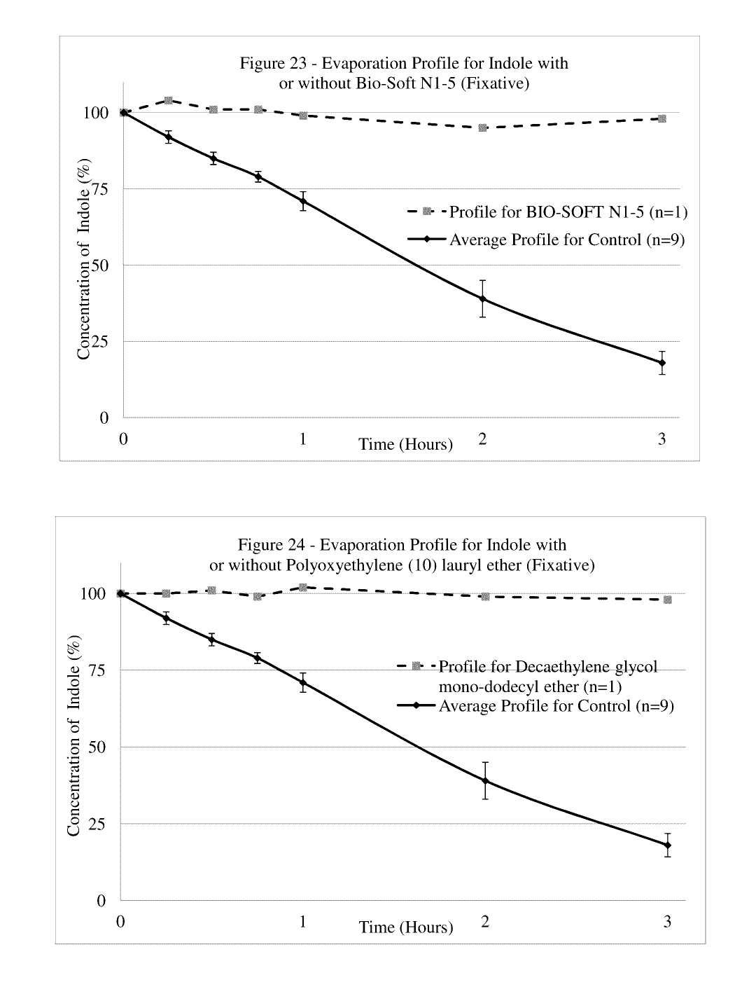

FIG. 23 shows the effect of the substantially non-odorous fragrance fixative Bio-soft N1-5 on the evaporation profile for a representative component (i.e., indole) of the test composition (MOD16). With reference to FIG. 23, indole has a difference (Δ) of 16% after 30 mins, 28% after 60 mins, and 80% after 3 hours. Addition of the Bio-soft N1-5 in the test composition (MOD16) maintains the concentration of the volatile fragrance material indole from 0 hour up to 3 hours whilst the control composition (REF), in the absence of Bio-soft N1-5, drops in fragrance profile concentration over the 3 hours. Thus, Bio-soft N1-5 acts to maintain the continued evaporation of the volatile fragrance material over time. Similar results are observed for the other volatile fragrance materials in the mixture (data not shown).

(q) Effects of Polyoxyethylene (10) Lauryl Ether on Composition Having Volatile Fragrance Materials

FIG. 24 shows the effect of the substantially non-odorous fragrance fixative Polyoxyethylene (10) lauryl ether on the evaporation profile for a representative component (i.e., indole) of the test composition (MOD17). With reference to FIG. 24, indole has a difference (A) of 16% after 30 mins, 31% after 60 mins, and 80% after 3 hours. Addition of the Polyoxyethylene (10) lauryl ether in the test composition (MOD17) maintains the concentration of the volatile fragrance material indole from 0 hour up to 3 hours whilst the control composition (REF), in the absence of Polyoxyethylene (10) lauryl ether, drops in fragrance concentration over the 3 hours. Thus, Polyoxyethylene (10) lauryl ether acts to maintain the continued evaporation of the volatile fragrance material over time. Similar results are observed for the other volatile fragrance materials in the mixture (data not shown).

(r) Effects of Arlamol™ PC10 on Composition Having Volatile Fragrance Materials

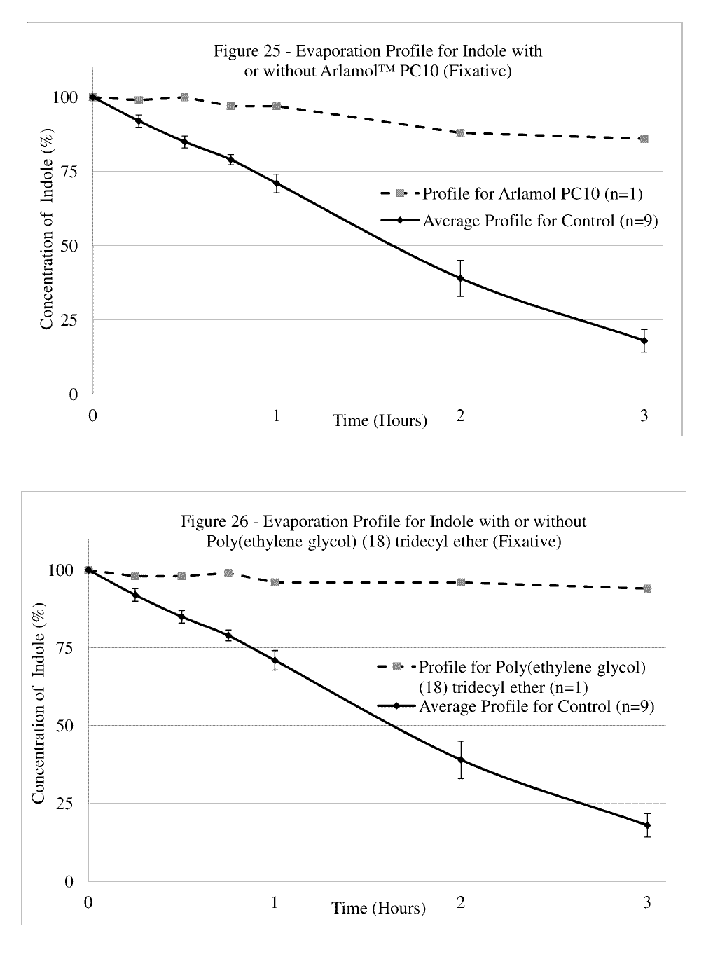

FIG. 25 shows the effect of the substantially non-odorous fragrance fixative Arlamol™ PC10 on the evaporation profile for a representative component (i.e., indole) of the test composition (MOD18). With reference to FIG. 25, indole has a difference (Δ) of 15% after 30 mins, 26% after 60 mins, and 68% after 3 hours. Addition of the Arlamol™ PC10 in the test composition (MOD18) maintains the concentration of the volatile fragrance material indole from 0 hour up to 3 hours whilst the control composition (REF), in the absence of Arlamol™ PC10, drops in fragrance concentration over the 3 hours. Thus, Arlamol™ PC10 acts to maintain the continued evaporation of the volatile fragrance material over time. Similar results are observed for the other volatile fragrance materials in the mixture (data not shown).

(s) Effects of Poly(Ethylene Glycol) (18) Tridecyl Ether on Composition Having Volatile Fragrance Materials

FIG. 26 shows the effect of the substantially non-odorous fragrance fixative Poly(ethylene glycol) (18) tridecyl ether on the evaporation profile for a representative component (i.e., indole) of the test composition (MOD19). With reference to FIG. 26, indole has a difference (Δ) of 13% after 30 mins, 25% after 60 mins, and 76% after 3 hours. Addition of the Poly(ethylene glycol) (18) tridecyl ether in the test composition (MOD19) maintains the concentration of the volatile fragrance material indole from 0 hour up to 3 hours whilst the control composition (REF), in the absence of Poly(ethylene glycol) (18) tridecyl ether, drops in fragrance concentration over the 3 hours. Thus, Poly(ethylene glycol) (18) tridecyl ether acts to maintain the continued evaporation of the volatile fragrance material over time. Similar results are observed for the other volatile fragrance materials in the mixture (data not shown).

(t) Effects of ALFONIC® 10-8 Ethoxylate on Composition Having Volatile Fragrance Materials

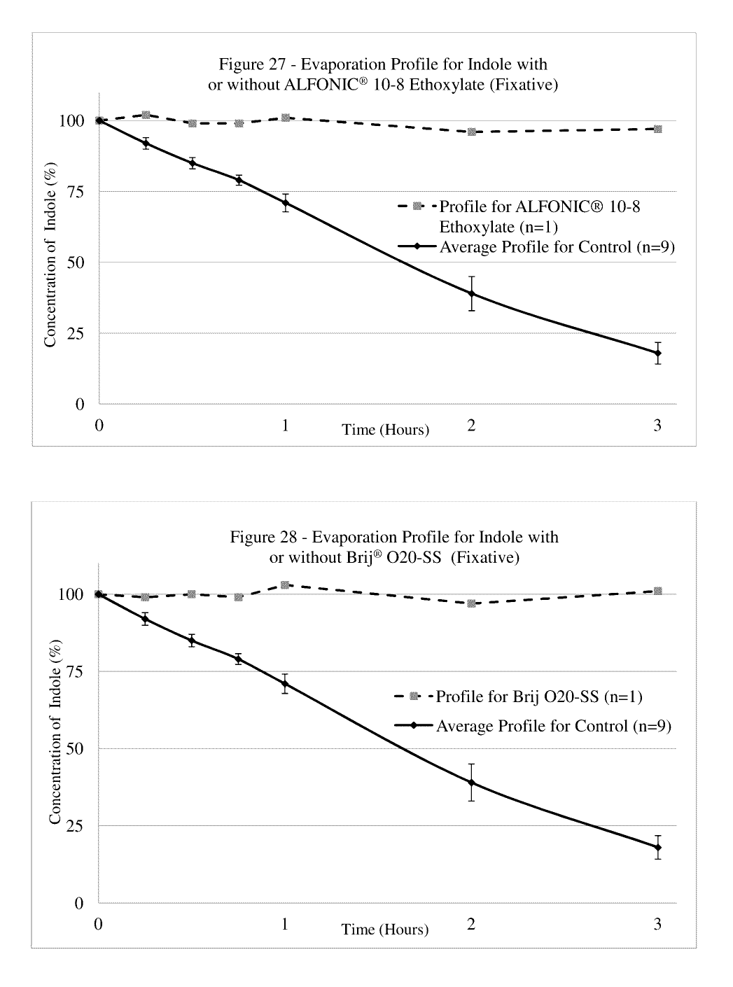

FIG. 27 shows the effect of the substantially non-odorous fragrance fixative ALFONIC® 10-8 Ethoxylate on the evaporation profile for a representative component (i.e., indole) of the test composition (MOD20). With reference to FIG. 27, indole has a difference (Δ) of 14% after 30 mins, 30% after 60 mins, and 79% after 3 hours. Addition of the Poly(ethylene glycol) (18) tridecyl ether in the test composition (MOD20) maintains the concentration of the volatile fragrance material indole from 0 hour up to 3 hours whilst the control composition (REF), in the absence of ALFONIC® 10-8 Ethoxylate, drops in fragrance concentration over the 3 hours. Thus, ALFONIC® 10-8 Ethoxylate acts to maintain the continued evaporation of the volatile fragrance material over time. Similar results are observed for the other volatile fragrance materials in the mixture (data not shown).

(u) Effects of Brij® O20-SS on Composition Having Volatile Fragrance Materials

FIG. 28 shows the effect of the substantially non-odorous fragrance fixative Brij® O20-SS on the evaporation profile for a representative component (i.e., indole) of the test composition (MOD21). With reference to FIG. 28, indole has a difference (Δ) of 15% after 30 mins, 32% after 60 mins, and 83% after 3 hours. Addition of the Brij® O20-SS in the test composition (MOD21) maintains the concentration of the volatile fragrance material indole from 0 hour up to 3 hours whilst the control composition (REF), in the absence of Brij® O20-SS, drops in fragrance concentration over the 3 hours. Thus, Brij® O20-SS acts to maintain the continued evaporation of the volatile fragrance material over time. Similar results are observed for the other volatile fragrance materials in the mixture (data not shown).

(v) Effects of Diethylene Glycol Butyl Ether on Composition Having Volatile Fragrance Materials

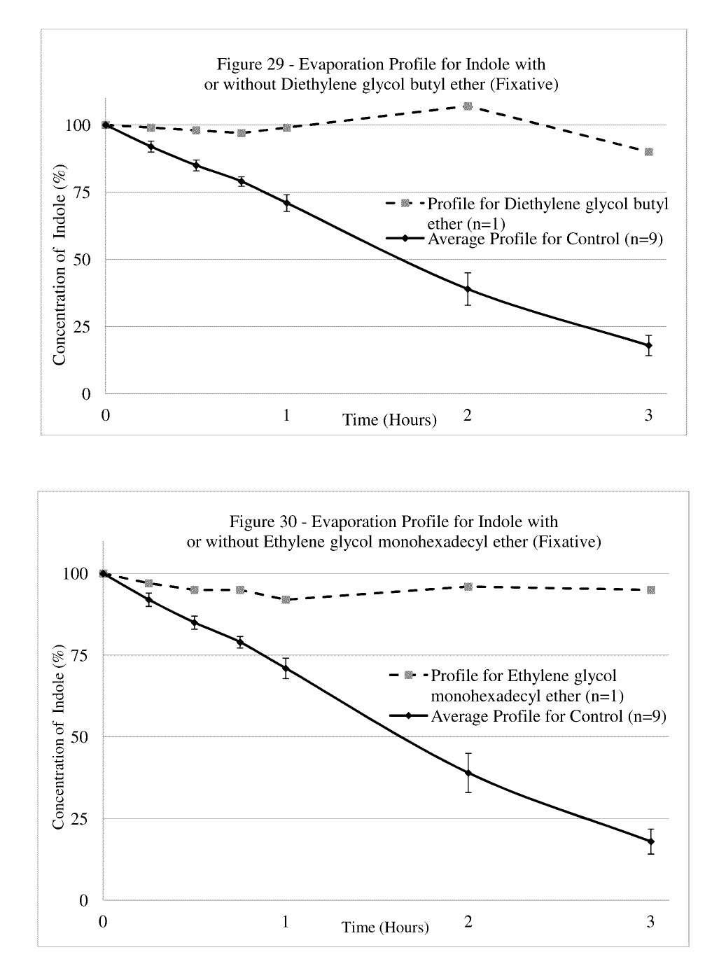

FIG. 29 shows the effect of the substantially non-odorous fragrance fixative Diethylene glycol butyl ether on the evaporation profile for a representative component (i.e., indole) of the test composition (MOD22). With reference to FIG. 29, indole has a difference (Δ) of 13% after 30 mins, 28% after 60 mins, and 72% after 3 hours. Addition of the Diethylene glycol butyl ether in the test composition (MOD22) maintains the concentration of the volatile fragrance material indole from 0 hour up to 3 hours whilst the control composition (REF), in the absence of Diethylene glycol butyl ether, drops in fragrance concentration over the 3 hours. Thus, Diethylene glycol butyl ether acts to maintain the continued evaporation of the volatile fragrance material over time. Similar results are observed for the other volatile fragrance materials in the mixture (data not shown).

(w) Effects of Ethylene Glycol Monohexadecyl Ether on Composition Having Volatile Fragrance Materials

FIG. 30 shows the effect of the substantially non-odorous fragrance fixative Ethylene glycol monohexadecyl ether on the evaporation profile for a representative component (i.e., indole) of the test composition (MOD23). With reference to FIG. 30, indole has a difference (A) of 10% after 30 mins, 21% after 60 mins, and 77% after 3 hours. Addition of the Ethylene glycol monohexadecyl ether in the test composition (MOD23) maintains the concentration of the volatile fragrance material indole from 0 hour up to 3 hours whilst the control composition (REF), in the absence of Ethylene glycol monohexadecyl ether, drops in fragrance concentration over the 3 hours. Thus, Ethylene glycol monohexadecyl ether acts to maintain the continued evaporation of the volatile fragrance material over time. Similar results are observed for the other volatile fragrance materials in the mixture (data not shown).

(x) Effects of Poly(Propylene Glycol) Monobutyl Ether on Composition Having Volatile Fragrance Materials

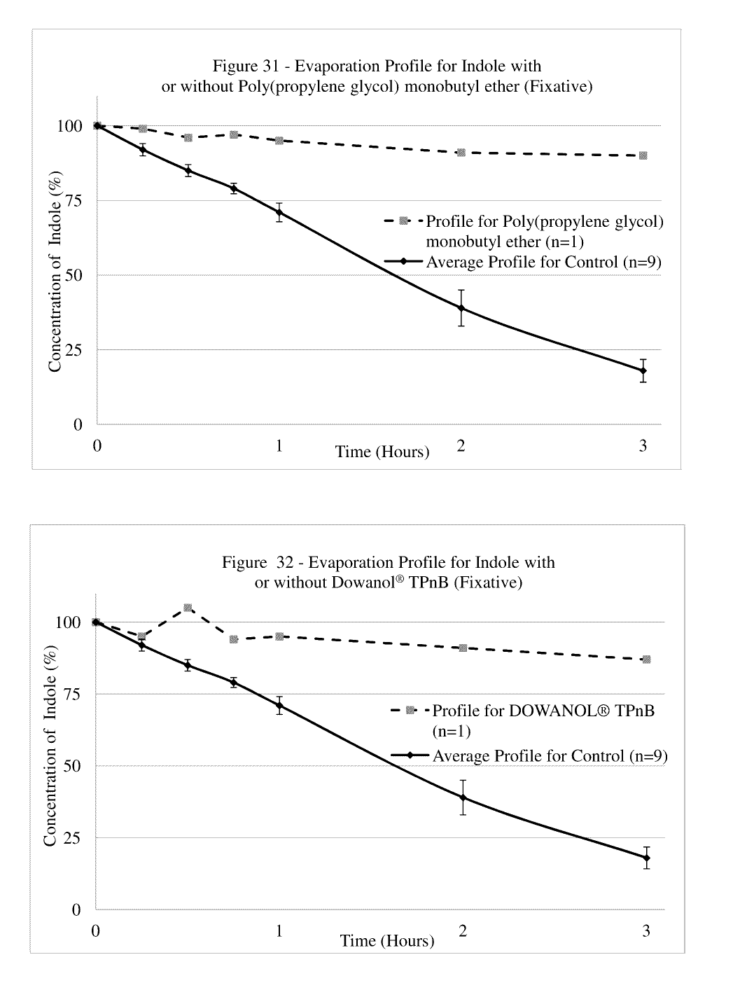

FIG. 31 shows the effect of the substantially non-odorous fragrance fixative Poly(propylene glycol) monobutyl ether on the evaporation profile for a representative component (i.e., indole) of the test composition (MOD24). With reference to FIG. 31, indole has a difference (Δ) of 11% after 30 mins, 24% after 60 mins, and 72% after 3 hours. Addition of the Poly(propylene glycol) monobutyl ether in the test composition (MOD24) maintains the concentration of the volatile fragrance material indole from 0 hour up to 3 hours whilst the control composition (REF), in the absence of Poly(propylene glycol) monobutyl ether, drops in fragrance concentration over the 3 hours. Thus, Poly(propylene glycol) monobutyl ether acts to maintain the continued evaporation of the volatile fragrance material over time. Similar results are observed for the other volatile fragrance materials in the mixture (data not shown).

(y) Effects of Dowanol™ TPnB on Composition Having Volatile Fragrance Materials

FIG. 32 shows the effect of the substantially non-odorous fragrance fixative Dowanol™ TPnB on the evaporation profile for a representative component (i.e., indole) of the test composition (MOD25). With reference to FIG. 32, indole has a difference (Δ) of 20% after 30 mins, 24% after 60 mins, and 69% after 3 hours. Addition of the Dowanol™ TPnB in the test composition (MOD25) maintains the concentration of the volatile fragrance material indole from 0 hour up to 3 hours whilst the control composition (REF), in the absence of Dowanol™ TPnB, drops in fragrance concentration over the 3 hours. Thus, Dowanol™ TPnB acts to maintain the continued evaporation of the volatile fragrance material over time. Similar results are observed for the other volatile fragrance materials in the mixture (data not shown).

(z) Effects of Tripropylene Glycol on Composition Having Volatile Fragrance Materials

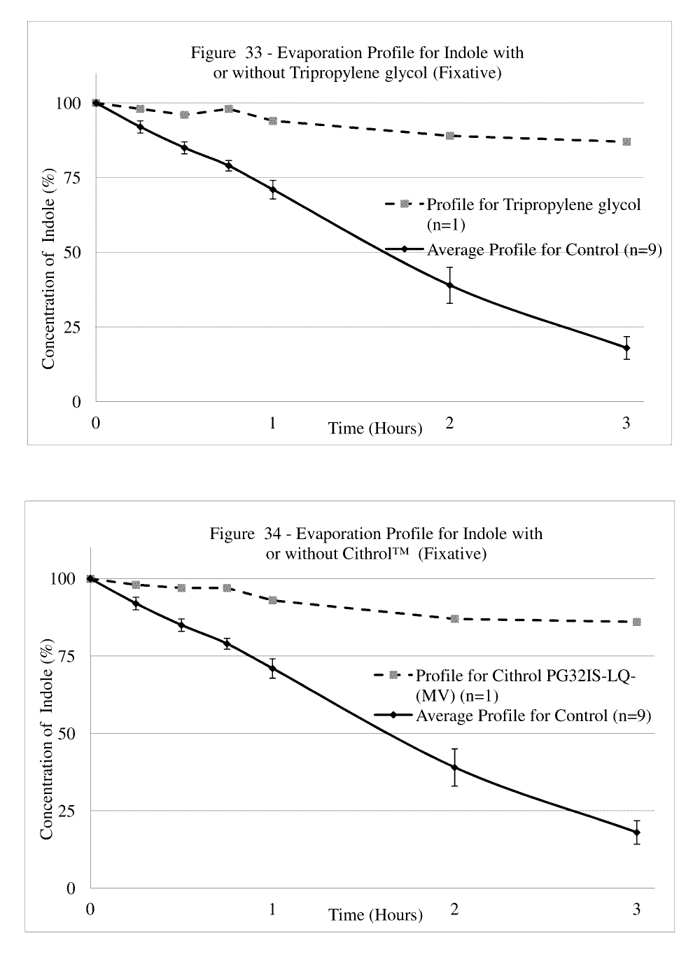

FIG. 33 shows the effect of the substantially non-odorous fragrance fixative Tripropylene Glycol on the evaporation profile for a representative component (i.e., indole) of the test composition (MOD26). With reference to FIG. 33, indole has a difference (Δ) of 11% after 30 mins, 23% after 60 mins, and 69% after 3 hours. Addition of the Tripropylene Glycol in the test composition (MOD26) maintains the concentration of the volatile fragrance material indole from 0 hour up to 3 hours whilst the control composition (REF), in the absence of Tripropylene Glycol, drops in fragrance concentration over the 3 hours. Thus, Tripropylene Glycol acts to maintain the continued evaporation of the volatile fragrance material over time. Similar results are observed for the other volatile fragrance materials in the mixture (data not shown).

(aa) Effects of Cithrol™ on Composition Having Volatile Fragrance Materials

FIG. 34 shows the effect of the substantially non-odorous fragrance fixative Cithrol™ on the evaporation profile for a representative component (i.e., indole) of the test composition (MOD27). With reference to FIG. 34, indole has a difference (Δ) of 12% after 30 mins, 22% after 60 mins, and 68% after 3 hours. Addition of the Cithrol™ in the test composition (MOD27) maintains the concentration of the volatile fragrance material indole from 0 hour up to 3 hours whilst the control composition (REF), in the absence of Cithrol™, drops in fragrance concentration over the 3 hours. Thus, Cithrol™ acts to maintain the continued evaporation of the volatile fragrance material over time. Similar results are observed for the other volatile fragrance materials in the mixture (data not shown).

(bb) Effects of Igepal® CO-630 on Composition Having Volatile Fragrance Materials

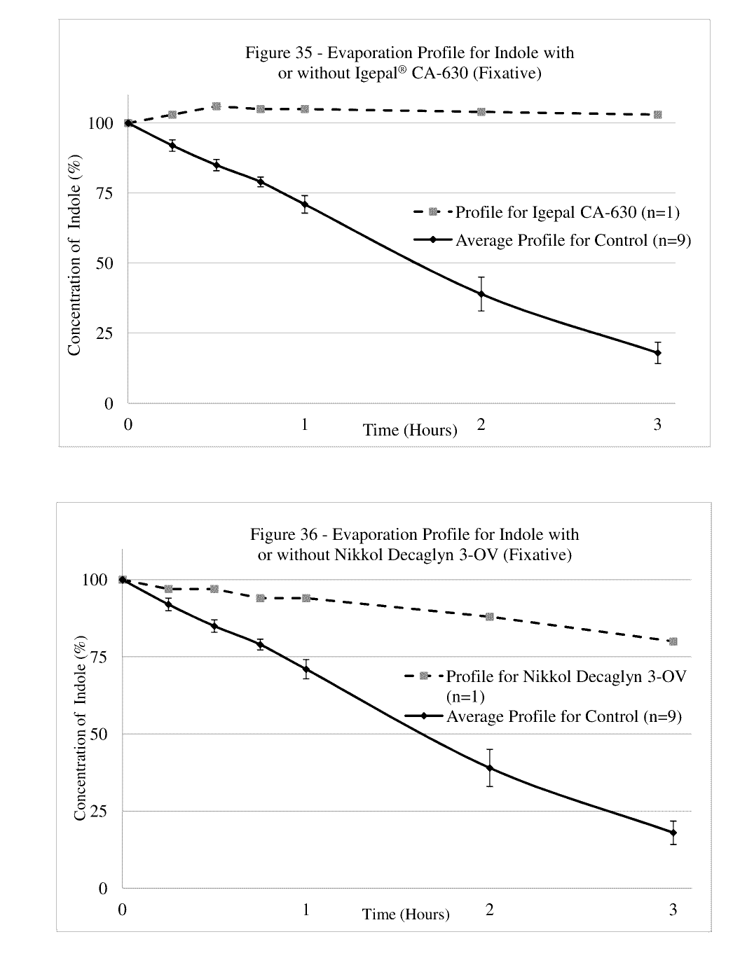

FIG. 35 shows the effect of the substantially non-odorous fragrance fixative Igepal® CO-630 on the evaporation profile for a representative component (i.e., indole) of the test composition (MOD28). With reference to FIG. 35, indole has a difference (Δ) of 21% after 30 mins, 34% after 60 mins, and 85% after 3 hours. Addition of the Igepal® CO-630 in the test composition (MOD28) maintains the concentration of the volatile fragrance material indole from 0 hour up to 3 hours whilst the control composition (REF), in the absence of Igepal® CO-630, drops in fragrance concentration over the 3 hours. Thus, Igepal® CO-630 acts to maintain the continued evaporation of the volatile fragrance material over time. Similar results are observed for the other volatile fragrance materials in the mixture (data not shown).

(cc) Effects of Nikkol Decaglyn 3-OV on Composition Having Volatile Fragrance Materials

FIG. 36 shows the effect of the substantially non-odorous fragrance fixative Nikkol Decaglyn 3-OV on the evaporation profile for a representative component (i.e., indole) of the test composition (MOD29). With reference to FIG. 36, indole has a difference (Δ) of 12% after 30 mins, 23% after 60 mins, and 62% after 3 hours. Addition of the Nikkol Decaglyn 3-OV in the test composition (MOD29) maintains the concentration of the volatile fragrance material indole from 0 hour up to 3 hours whilst the control composition (REF), in the absence of Nikkol Decaglyn 3-OV, drops in fragrance concentration over the 3 hours. Thus, Nikkol Decaglyn 3-OV acts to maintain the continued evaporation of the volatile fragrance material over time. Similar results are observed for the other volatile fragrance materials in the mixture (data not shown).

(dd) Effects of NIKKOL Hexaglyn 1-L on Composition Having Volatile Fragrance Materials

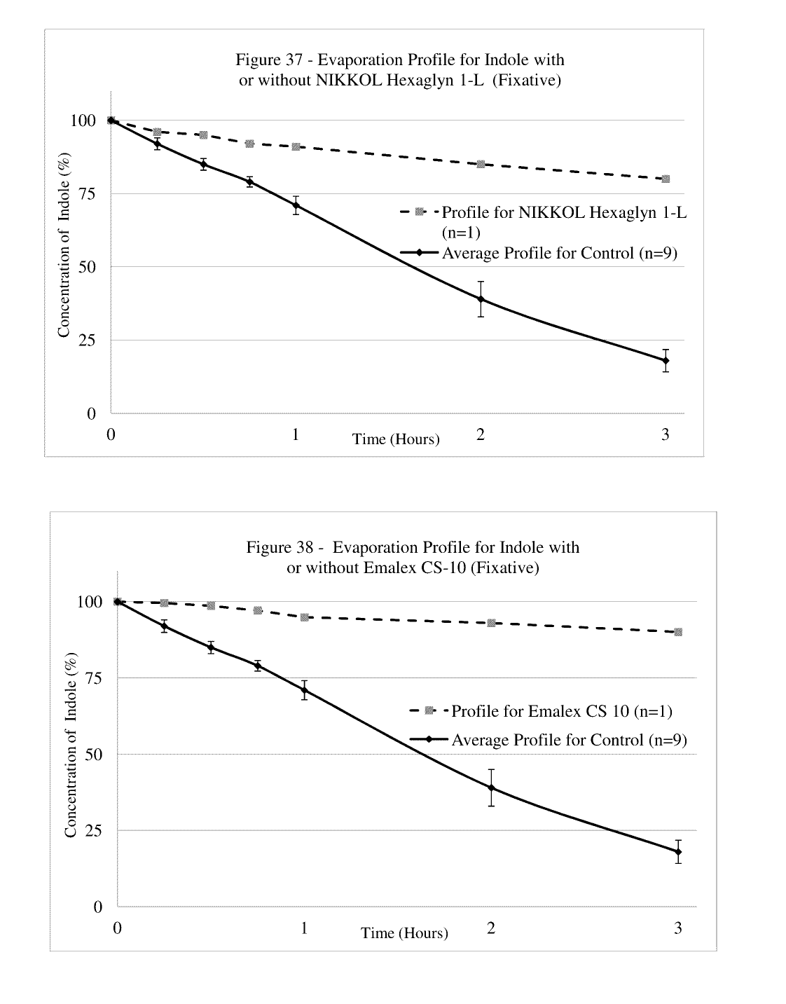

FIG. 37 shows the effect of the substantially non-odorous fragrance fixative NIKKOL Hexaglyn 1-L on the evaporation profile for a representative component (i.e., indole) of the test composition (MOD30). With reference to FIG. 37, indole has a difference (Δ) of 10% after 30 mins, 20% after 60 mins, and 62% after 3 hours. Addition of the NIKKOL Hexaglyn 1-L in the test composition (MOD30) maintains the concentration of the volatile fragrance material indole from 0 hour up to 3 hours whilst the control composition (REF), in the absence of NIKKOL Hexaglyn 1-L, drops in fragrance concentration over the 3 hours. Thus, NIKKOL Hexaglyn 1-L acts to maintain the continued evaporation of the volatile fragrance material over time. Similar results are observed for the other volatile fragrance materials in the mixture (data not shown).

(ee) Effects of Emalex CS-10 on Composition Having Volatile Fragrance Materials

FIG. 38 shows the effect of the substantially non-odorous fragrance fixative Emalex CS-10 on the evaporation profile for a representative component (i.e., indole) of the test composition (MOD31). With reference to FIG. 38, indole has a difference (Δ) of 14% after 30 mins, 24% after 60 mins, and 72% after 3 hours. Addition of the Emalex CS-10 in the test composition (MOD31) maintains the concentration of the volatile fragrance material indole from 0 hour up to 3 hours whilst the control composition (REF), in the absence of Emalex CS-10, drops in fragrance concentration over the 3 hours. Thus, Emalex CS-10 acts to maintain the continued evaporation of the volatile fragrance material over time. Similar results are observed for the other volatile fragrance materials in the mixture (data not shown).

(ff) Effects of Dioctyl Ether on Composition Having Volatile Fragrance Materials

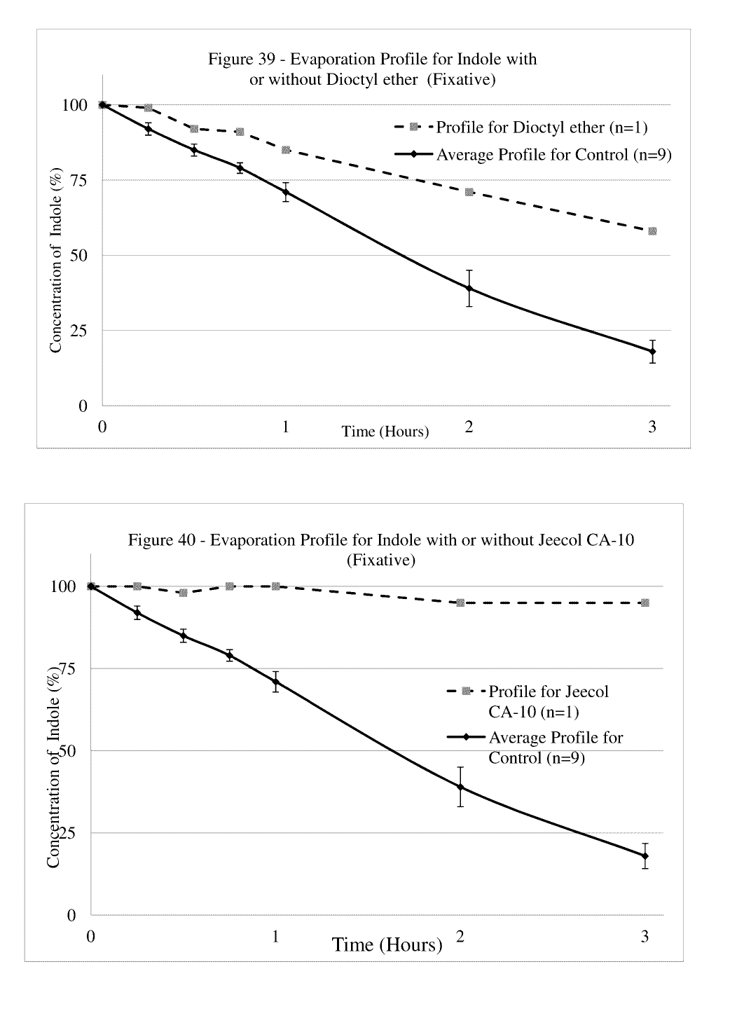

FIG. 39 shows the effect of the substantially non-odorous fragrance fixative Dioctyl ether on the evaporation profile for a representative component (i.e., indole) of the test composition (MOD32). With reference to FIG. 39, indole has a difference (Δ) of 7% after 30 mins, 14% after 60 mins, and 40% after 3 hours. Addition of the Dioctyl ether in the test composition (MOD32) maintains the concentration of the volatile fragrance material indole from 0 hour up to 3 hours whilst the control composition (REF), in the absence of Dioctyl ether, drops in fragrance concentration over the 3 hours. Thus, Dioctyl ether acts to maintain the continued evaporation of the volatile fragrance material over time. Similar results are observed for the other volatile fragrance materials in the mixture (data not shown).

(gg) Effects of Jeecol CA-10 on Composition Having Volatile Fragrance Materials

FIG. 40 shows the effect of the substantially non-odorous fragrance fixative Jeecol CA-10 on the evaporation profile for a representative component (i.e., indole) of the test composition (MOD33). With reference to FIG. 40, indole has a difference (Δ) of 13% after 30 mins, 29% after 60 mins, and 77% after 3 hours. Addition of the Jeecol CA-10 in the test composition (MOD33) maintains the concentration of the volatile fragrance material indole from 0 hour up to 3 hours whilst the control composition (REF), in the absence of Jeecol CA-10, drops in fragrance concentration over the 3 hours. Thus, Jeecol CA-10 acts to maintain the continued evaporation of the volatile fragrance material over time. Similar results are observed for the other volatile fragrance materials in the mixture (data not shown).

(hh) Effects of Steareth-10 on Composition Having Volatile Fragrance Materials

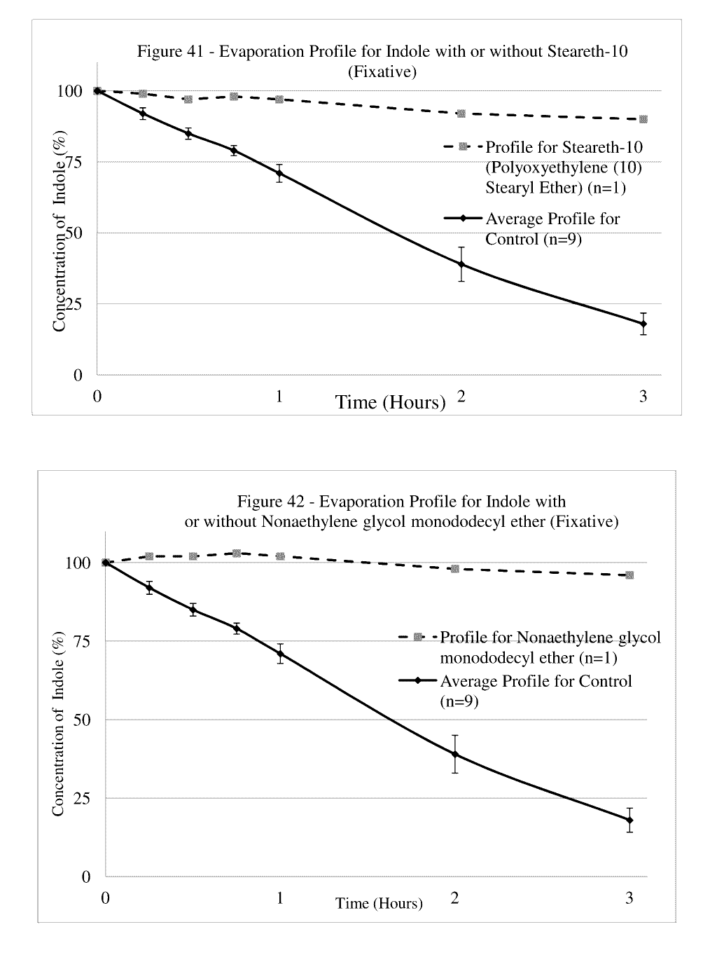

FIG. 41 shows the effect of the substantially non-odorous fragrance fixative Steareth-10 on the evaporation profile for a representative component (i.e., indole) of the test composition (MOD34). With reference to FIG. 41, indole has a difference (Δ) of 12% after 30 mins, 26% after 60 mins, and 72% after 3 hours. Addition of the Steareth-10 in the test composition (MOD34) maintains the concentration of the volatile fragrance material indole from 0 hour up to 3 hours whilst the control composition (REF), in the absence of Steareth-10, drops in fragrance concentration over the 3 hours. Thus, Steareth-10 acts to maintain the continued evaporation of the volatile fragrance material over time. Similar results are observed for the other volatile fragrance materials in the mixture (data not shown).

(ii) Effects of Nonaethylene Glycol Monododecyl Ether on Composition Having Volatile Fragrance Materials

FIG. 42 shows the effect of the substantially non-odorous fragrance fixative Nonaethylene glycol monododecyl ether on the evaporation profile for a representative component (i.e., indole) of the test composition (MOD35). With reference to FIG. 42, indole has a difference (Δ) of 17% after 30 mins, 31% after 60 mins, and 78% after 3 hours. Addition of the Nonaethylene glycol monododecyl ether in the test composition (MOD35) maintains the concentration of the volatile fragrance material indole from 0 hour up to 3 hours whilst the control composition (REF), in the absence of Nonaethylene glycol monododecyl ether, drops in fragrance concentration over the 3 hours. Thus, Nonaethylene glycol monododecyl ether acts to maintain the continued evaporation of the volatile fragrance material over time. Similar results are observed for the other volatile fragrance materials in the mixture (data not shown).

(jj) Effects of Glycerol Propoxylate on Composition Having Volatile Fragrance Materials

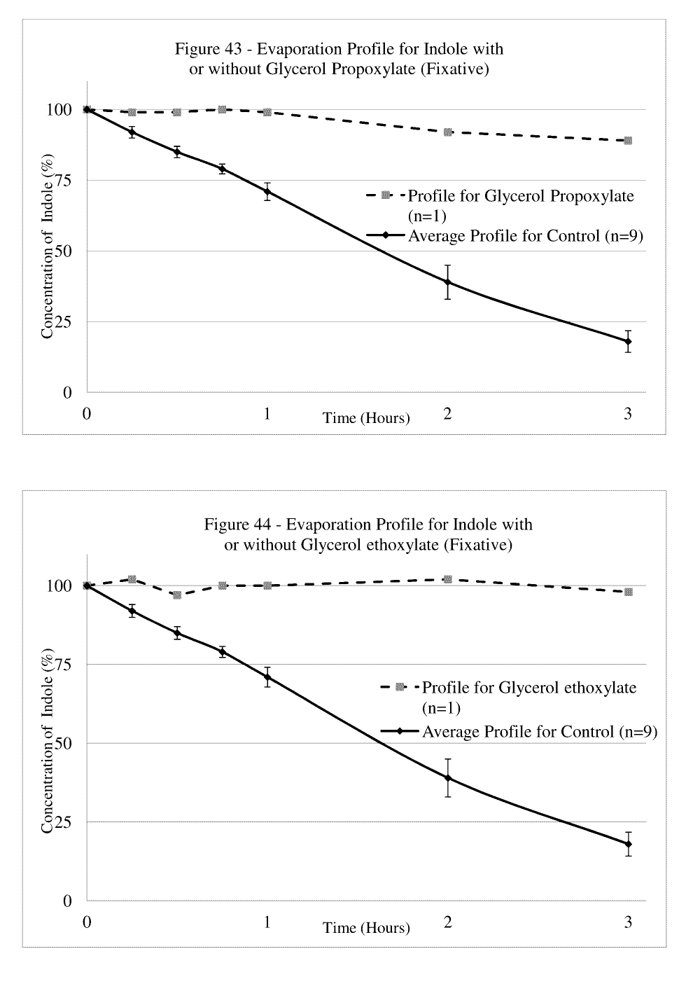

FIG. 43 shows the effect of the substantially non-odorous fragrance fixative Glycerol propoxylate on the evaporation profile for a representative component (i.e., indole) of the test composition (MOD36). With reference to FIG. 43, indole has a difference (Δ) of 14% after 30 mins, 28% after 60 mins, and 71% after 3 hours. Addition of the Glycerol propoxylate in the test composition (MOD36) maintains the concentration of the volatile fragrance material indole from 0 hour up to 3 hours whilst the control composition (REF), in the absence of Glycerol propoxylate, drops in fragrance concentration over the 3 hours. Thus, Glycerol propoxylate acts to maintain the continued evaporation of the volatile fragrance material over time. Similar results are observed for the other volatile fragrance materials in the mixture (data not shown).

(kk) Effects of Glycerol Ethoxylate on Composition Having Volatile Fragrance Materials

FIG. 44 shows the effect of the substantially non-odorous fragrance fixative Glycerol ethoxylate on the evaporation profile for a representative component (i.e., indole) of the test composition (MOD37). With reference to FIG. 44, indole has a difference (Δ) of 12% after 30 mins, 29% after 60 mins, and 80% after 3 hours. Addition of the Glycerol ethoxylate in the test composition (MOD37) maintains the concentration of the volatile fragrance material indole from 0 hour up to 3 hours whilst the control composition (REF), in the absence of Glycerol ethoxylate, drops in fragrance concentration over the 3 hours. Thus, Glycerol ethoxylate acts to maintain the continued evaporation of the volatile fragrance material over time. Similar results are observed for the other volatile fragrance materials in the mixture (data not shown).

(ll) Effects of Hexaethylene Glycol Monohexadecyl Ether on Composition Having Volatile Fragrance Materials

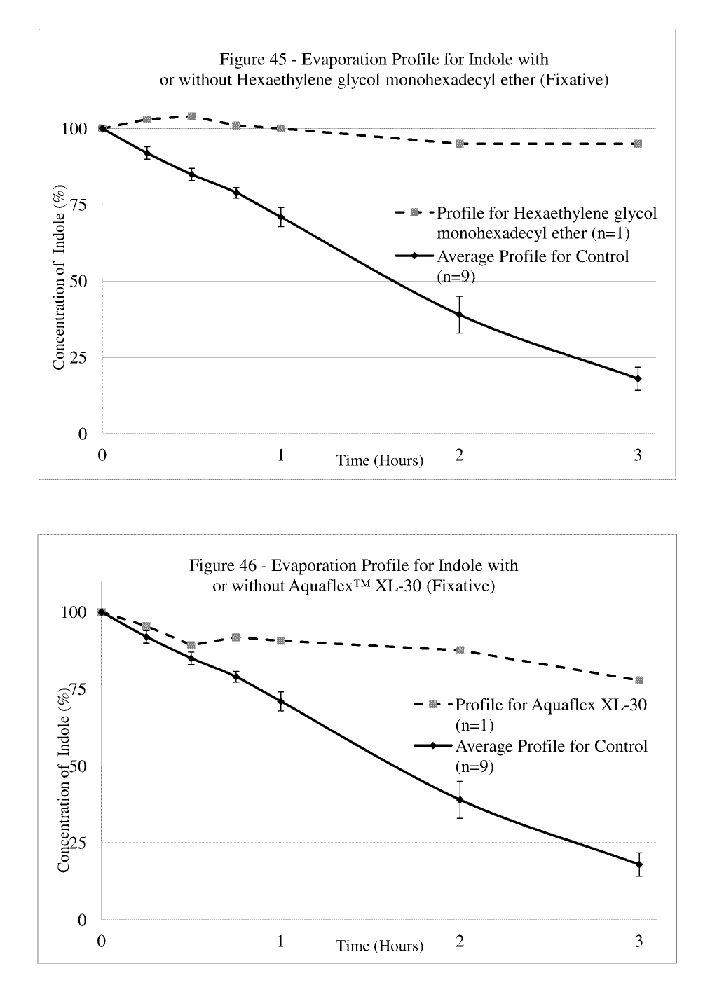

FIG. 45 shows the effect of the substantially non-odorous fragrance fixative Hexaethylene glycol monohexadecyl ether on the evaporation profile for a representative component (i.e., indole) of the test composition (MOD38). With reference to FIG. 45, indole has a difference (Δ) of 19% after 30 mins, 29% after 60 mins, and 77% after 3 hours. Addition of the Hexaethylene glycol monohexadecyl ether in the test composition (MOD38) maintains the concentration of the volatile fragrance material indole from 0 hour up to 3 hours whilst the control composition (REF), in the absence of Hexaethylene glycol monohexadecyl ether, drops in fragrance concentration over the 3 hours. Thus, Hexaethylene glycol monohexadecyl ether acts to maintain the continued evaporation of the volatile fragrance material over time. Similar results are observed for the other volatile fragrance materials in the mixture (data not shown).

(mm) Effects of Aquaflex™ XL-30 on Composition Having Volatile Fragrance Materials

FIG. 46 shows the effect of the substantially non-odorous fragrance fixative Aquaflex™ XL-30 on the evaporation profile for a representative component (i.e., indole) of the test composition (MOD39). With reference to FIG. 46, indole has a difference (Δ) of 4% after 30 mins, 20% after 60 mins, and 60% after 3 hours. Addition of the Aquaflex™ XL-30 in the test composition (MOD39) maintains the concentration of the volatile fragrance material indole from 0 hour up to 3 hours whilst the control composition (REF), in the absence of Aquaflex™ XL-30, drops in fragrance concentration over the 3 hours. Thus, Aquaflex™ XL-30 acts to maintain the continued evaporation of the volatile fragrance material over time. Similar results are observed for the other volatile fragrance materials in the mixture (data not shown).

(nn) Effects of Piperonyl Butoxide on Composition Having Volatile Fragrance Materials

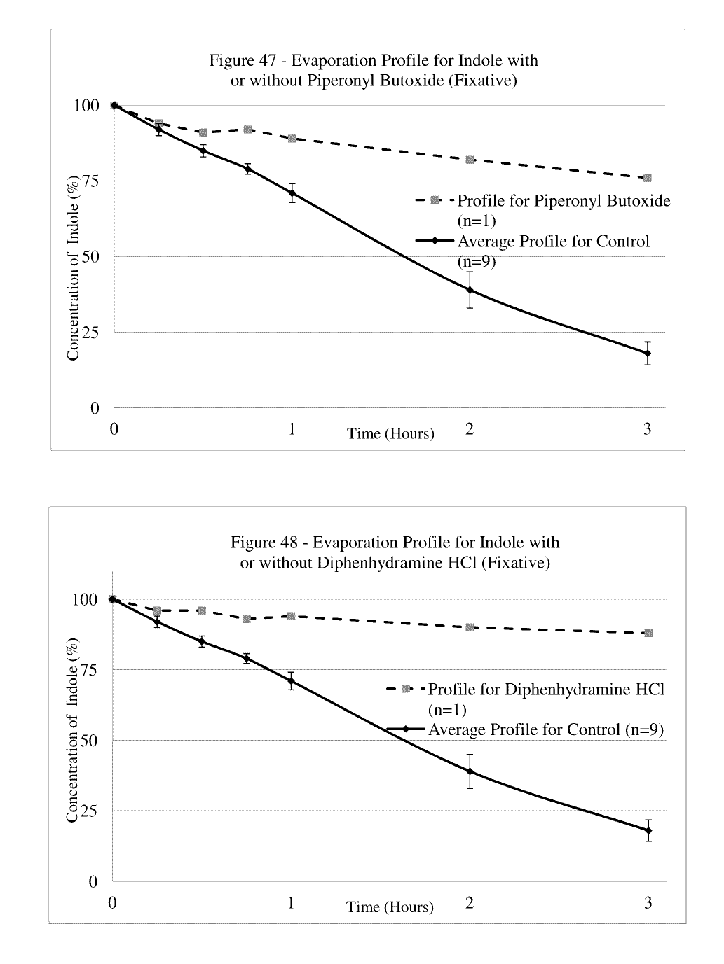

FIG. 47 shows the effect of the substantially non-odorous fragrance fixative Piperonyl Butoxide on the evaporation profile for a representative component (i.e., indole) of the test composition (MOD40). With reference to FIG. 47, indole has a difference (Δ) of 6% after 30 mins, 18% after 60 mins, and 58% after 3 hours. Addition of the Piperonyl Butoxide in the test composition (MOD40) maintains the concentration of the volatile fragrance material indole from 0 hour up to 3 hours whilst the control composition (REF), in the absence of Piperonyl Butoxide, drops in fragrance concentration over the 3 hours. Thus, Piperonyl Butoxide acts to maintain the continued evaporation of the volatile fragrance material over time. Similar results are observed for the other volatile fragrance materials in the mixture (data not shown).

(oo) Effects of Diphenhydramine HCl on Composition Having Volatile Fragrance Materials

FIG. 48 shows the effect of the substantially non-odorous fragrance fixative Diphenhydramine HCl on the evaporation profile for a representative component (i.e., indole) of the test composition (MOD41). With reference to FIG. 48, indole has a difference (Δ) of 11% after 30 mins, 23% after 60 mins, and 70% after 3 hours. Addition of the Diphenhydramine HCl in the test composition (MOD41) maintains the concentration of the volatile fragrance material indole from 0 hour up to 3 hours whilst the control composition (REF), in the absence of Diphenhydramine HCl, drops in fragrance concentration over the 3 hours. Thus, Diphenhydramine HCl acts to maintain the continued evaporation of the volatile fragrance material over time. Similar results are observed for the other volatile fragrance materials in the mixture (data not shown).

(pp) Effect of Di(Propylene Glycol) Propyl Ether on Composition Having Volatile Fragrance Materials

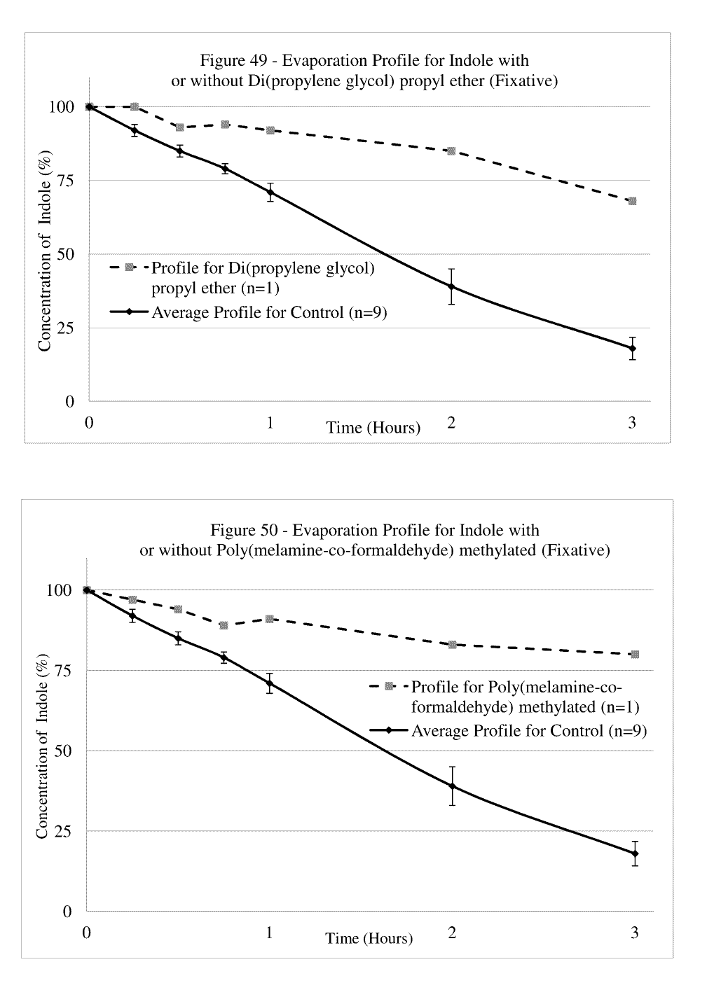

FIG. 49 shows the effect of the substantially non-odorous fragrance fixative Di(propylene glycol) propyl ether on the evaporation profile for a representative component (i.e., indole) of the test composition (MOD42). With reference to FIG. 49, indole has a difference (A) of 8% after 30 mins, 21% after 60 mins, and 50% after 3 hours. Addition of the Di(propylene glycol) propyl ether in the test composition (MOD42) maintains the concentration of the volatile fragrance material indole from 0 hour up to 3 hours whilst the control composition (REF), in the absence of Di(propylene glycol) propyl ether, drops in fragrance concentration over the 3 hours. Thus, Di(propylene glycol) propyl ether acts to maintain the continued evaporation of the volatile fragrance material over time. Similar results are observed for the other volatile fragrance materials in the mixture (data not shown).

(qq) Effects of Poly(Melamine-Co-Formaldehyde) Methylated on Composition Having a Volatile Fragrance Materials

FIG. 50 shows the effect of the substantially non-odorous fragrance fixative Poly(melamine-co-formaldehyde) methylated on the evaporation profile for a representative component (i.e., indole) of the test composition (MOD43). With reference to FIG. 50, indole has a difference (Δ) of 9% after 30 mins, 20% after 60 mins, and 62% after 3 hours. Addition of the Poly(melamine-co-formaldehyde) methylated in the test composition (MOD43) maintains the concentration of the volatile fragrance material indole from 0 hour up to 3 hours whilst the control composition (REF), in the absence of Poly(melamine-co-formaldehyde) methylated, drops in fragrance concentration over the 3 hours. Thus, Poly(melamine-co-formaldehyde) methylated acts to maintain the continued evaporation of the volatile fragrance material over time. Similar results are observed for the other volatile fragrance materials in the mixture (data not shown).

### Example 7

### Analytical Headspace Test Results

Using the analytical headspace Test Method 4, it is possible to demonstrate the character retention over time of a perfume mixture of a fragrance composition of the present invention vs. a control. Compositions disclosed in Table 19(a) are added to sealed vials in accordance with the procotol described in the Method Section, and the fragrance profile in the headspace are measured at specific time points through the use of headspace gas chromatography.

(a) Effects of the Substantially Non-Odorous Fragrance Fixatives on Character Retention of Compositions Having Reduced Levels of Low Volatile Fragrance Materials (Between 10 to 30 wt % Relative to the Total Weight of the Fragrance Component) Vs. Compositions Having Traditional Levels of Low Volatile Fragrance Materials (Greater than 30 wt % Relative to the Total Weight of the Fragrance Component)

The test demonstrates the character retention over time of a fragrance composition. The results show the effect of the substantially non-odorous fragrance fixative and reduced levels of low volatile fragrance materials for any one of the inventive Compositions A1 on fragrance profile longevity versus control Compositions C1 in the absence of the substantially non-odorous fixative. Alternatively, results show the effect of the substantially non-odorous fragrance fixative and reduced levels of low volatile fragrance materials for any one of the inventive Compositions A1 on fragrance profile longevity versus traditional Compositions B1 in the presence of the substantially non-odorous fragrance fixative. Fragrance profile fidelity, particularly characters attributable to the volatile fragrance materials are maintained for up to at least 1 hour in the presence of the substantially non-odorous fragrance fixative whilst it drops in the absence of the substantially non-odorous fragrance fixative.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
(i) a fragrance component present in an amount of from about 0.04 wt % to about 30 wt %, relative to the total weight of the composition; and
(ii) at least one substantially non-odorous fragrance fixative from the group consisting of the materials in Table 1, wherein the substantially non-odorous fragrance fixative is present in the amount of from about 0.1 wt % to about 20 wt %, relative to the total weight of the composition.

2. The composition according to claim 1, wherein:
(i) the fragrance component is present from about 0.04 wt % to about 30 wt %, relative to the total weight of composition, and wherein the fragrance component comprises:
(a) at least one low volatile fragrance material having a vapor pressure less than 0.001 Torr (0.000133 kPa) at 25° C.; and
(b) the low volatile fragrance material is present in an amount of less than about 30 wt %, relative to the total weight of the fragrance component; and
(ii) at least one substantially non-odorous fragrance fixative present in the amount of from about 0.1 wt % to about 20 wt %, relative to the total weight of the composition.

3. The composition according to claim 2, wherein the low volatile fragrance material is present in an amount of from about 10 wt % to about 30 wt %, relative to the total weight of the fragrance component.

4. The composition according to claim 2, wherein the low volatile fragrance material is selected from at least 1 material, or at least 2 materials, or at least 3 materials from the group of Low Volatile Fragrance Materials 1-111, IUPAC Names, of Table 2 and mixtures thereof.

5. The composition according to claim 4, wherein the low volatile fragrance material is selected from the group of Table 2 Low Volatile Fragrance Materials 1, 4-6, 8, 12-16, 18, 22-28, 31, 34-37, 41, 45, 47, 52-55, 57, 60, 61, 63, 65, 68, 69-73, 75, 78, 80, 83-84, 89, 94, 99, 102, 104, 106-108, and mixtures thereof.

6. The composition according to claim 2, wherein:
(i) the fragrance component further comprising one or more volatile fragrance materials, wherein:
(c) the volatile fragrance material has a vapor pressure greater than or equal to 0.001 Torr (0.000133 kPa) at 25° C.;
(d) the volatile fragrance material is present in an amount of from about 70 wt % to about 99.9 wt %, relative to the total weight of the fragrance component; and
(e) mixtures thereof.

7. The composition according to claim 6, wherein the volatile fragrance material is selected from at least 1 material, or at least 3 materials, or at least 5 materials from the group of Volatile Fragrance Materials 1-449, IUPAC Names, of Table 3 and mixtures thereof.

8. The composition according to claim 7, wherein the volatile fragrance material is selected from the group of Table 3 Volatile Fragrance Materials 4, 6, 18, 54, 60, 77, 92, 105, 107, 119, 122, 134, 138, 141, 144, 148, 152, 155, 158, 167, 173, 175, 176, 182, 186, 189, 193, 195, 196, 202, 205, 206, 207, 210, 212, 225, 235, 238, 243, 245, 257, 271, 273, 274, 279-280, 282, 285, 289, 293, 297, 302, 305-306, 314, 315, 318, 354-355, 410, 416, and mixtures thereof.

9. The composition according to claim 6, wherein the volatile fragrance material is selected from the group consisting of:
(f) a high volatile fragrance material having a vapor pressure greater than 0.1 Torr (0.0133 kPa) at 25° C., present in an amount of from about 1 wt % to about 30 wt %, relative to the total weight of the fragrance component;
(g) a moderate volatile fragrance material having a vapor pressure in the range of 0.1 Torr (0.0133 kPa) to 0.001 Torr (0.000133 kPa) at 25° C., present in an amount of from about 40 wt % to about 80 wt %, relative to the total weight of the fragrance component; and
(h) mixtures thereof.

10. The composition according to claim 1, further comprising a volatile solvent present in the amount of from about 10 wt % to about 90 wt %, relative to the total weight of the composition, and wherein the solvent is a branch or unbranched C₁ to C₁₀ alkyl, akenyl or alkynyl group having at least one alcohol moiety.

11. The composition according to claim 1, wherein the composition is a fine fragrance composition, preferably in the form of a perfume concentrate, a perfume, a parfum, an eau de toilette, an eau de parfum or a cologne.

12. The composition according to claim 1, wherein the composition is in the form of a body splash or a body spray.

13. The composition according to claim 1, wherein the substantially non-odorous fragrance fixative and fragrance component are present in a weight ratio from about 10:1 to about 1:10.

14. The composition according to claim 1, wherein the substantially non-odorous fragrance fixative is selected from the group consisting of Table 1 substantially non-odorous fragrance fixatives 1-190, 191 and mixtures thereof.

15. A method of modifying or enhancing the odour properties of a substrate, comprising contacting or treating the substrate with a composition according to claim 1.

16. A substantially non-odorous fragrance fixative for fragrance materials, wherein the substantially non-odorous fragrance fixative comprises at least one material selected from the group consisting of the materials in Table 1.

17. A perfuming consumer product or article comprising a composition according to claim 1, wherein the perfuming consumer product is selected from the group consisting of a fabric care product, an air care product or a home care product.
